(19) United States
(12) Patent Application Publication
(10) Pub. No.: US 2021/0052428 A1
(43) Pub. Date: Feb. 25, 2021

(54) DESIGNS FOR TYMPANOSTOMY CONDUITS OR SUBANNULAR VENTILATION CONDUITS AND OTHER MEDICAL AND FLUIDIC CONDUITS

(71) Applicants: President and Fellows of Harvard College, Cambridge, MA (US); Massachusetts Eye and Ear Infirmary, Boston, MA (US)

(72) Inventors: Nicole Leah BLACK, Somerville, MA (US); Ida PAVLICHENKO, Watertown, MA (US); Michael J. KREDER, Mississauga (CA); Elliott D. KOZIN, Boston, MA (US); Aaron Kyle REMENSCHNEIDER, Boston, MA (US); Joanna AIZENBERG, Boston, MA (US); Haritosh PATEL, Brampton (CA)

(21) Appl. No.: 16/982,445

(22) PCT Filed: Mar. 20, 2019
(86) PCT No.: PCT/US19/23276

§ 371 Date: Sep. 18, 2020

Related U.S. Application Data
(60) Provisional application No. 62/645,629, filed on Mar. 20, 2018.

(51) Int. Cl. A61F 11/00; A61L 31/04; A61L 31/14

(52) U.S. Cl. CPC A61F 11/002; A61L 31/04; A61L 31/145; A61L 2300/414; A61L 2300/41; A61L 2300/22; A61L 2300/406

(57) ABSTRACT

A system includes a device having a conduit having a proximal end having a proximal end radius, a distal end opposite the proximal end and having distal end radius, an inner surface connecting the proximal end and the distal end and forming a proximal angle at the ends, the inner surface having surface properties, and an outer surface connecting the ends; the distal end radius, the proximal end radius, the distal angle, the proximal angle, and the surface properties of the inner surface are selected to: allow entry of a first material to the distal, transport of the first material through the conduit along the inner surface toward the proximal end, and exit of the first material from the proximal end, and to resist entry of a second material into the proximal end; and the Young-Laplace pressure for the first material is less for the second material.

### COPYRIGHT NOTICE

This patent disclosure can contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION BY REFERENCE

All patents, patent applications and publications cited herein are hereby incorporated by reference in their entirety in order to more fully describe the state of the art as known to those skilled therein as of the date of the invention described herein.

### FIELD OF THE INVENTION

The present application relates to conduits that can be used for medical applications, such as tympanostomy conduits and subannular ventilation conduits, or for non-medical applications. More particularly, the present application relates to conduits with anti-fouling properties, guided fluid transport, minimal invasiveness, and/or programmable shape and chemistry information.

### BACKGROUND

### I. Incidence and Impact of Otitis Media

Acute otitis media (AOM), also known as an ear infection, and otitis media with effusion (OME) are the leading causes of healthcare visits worldwide. Otitis media (OM) occurs in the middle ear space behind the eardrum, usually after a cold or other upper respiratory infection has been present for several days. During this infection, the Eustachian tubes swell, preventing air from entering the middle ear and pulling fluid into the middle ear space. This trapped fluid, containing mucins, harbors bacteria and viruses.

### II. Placement of Tympanostomy Tubes and Subannular Ventilation Tubes

Acute otitis media (AOM), also known as an ear infection, and otitis media with effusion (OME) are the leading causes of healthcare visits worldwide, and lead to considerable patient morbidity and significant annual healthcare burden of >$5B of direct and indirect costs in the US. Globally, AOM affects over 700 million people each year; children tend to be disproportionately affected relative to adults with estimates of global incidence peaking at 61% in ages 1-4. AOM, is the most common infection in pediatric patients, affecting over 8.8 million U.S. children and causing 12 to 16 million physician visits per year in the US. Acute OM has a prevalence of 60% within the first 5 years of life. OM occurs in the middle ear space behind the eardrum, usually after a cold or other upper respiratory infection has been present for several days. During this infection, the Eustachian tubes swell, preventing air from entering the middle ear and pulling fluid into the middle ear space. This trapped fluid, containing mucins, harbors bacteria and viruses. Since children younger than age 7 have shorter and more horizontal Eustachian tubes, these become blocked more easily, leading to a higher occurrence of ear infections.

Left untreated, OM can lead to symptoms including pain, fever, vomiting, loss of appetite, difficulty sleeping, dizziness, recurrent acute infections, hearing loss, and speech delays. Severe complications of acute OM include disabling acute mastoiditis, subperiosteal abscess, intracranial suppuration, meningitis, and facial nerve palsy. In the developing world, chronic OM frequently results in these permanent hearing sequelae, and when untreated, is estimated to result in more than 28,000 deaths worldwide due to the aforementioned complications according to a WHO report.

A total of $2.8 billion was spent on treatment of OM in 2006, not including over-the-counter medications. The current standard of care consists of a 10-day course of broad spectrum oral antibiotics. OM is the most common reason for prescribing antibiotics to US children. Treatment of acute otitis media in children under 2 years of age. Thus, OM treatment is believed to add to the ongoing increase in antibiotic resistance among pathogenic bacteria. Systemic antibiotic administration often results in side effects, including diarrhea, dermatitis, vomiting, and oral thrush. Even after the middle ear space is no longer infected, fluid can remain in the ear. Approximately 30% of children still have fluid in the middle ear one month after an ear infection and 20% still have fluid after two months. This fluid causes recurrent infections, with 40% of children having 4 or more episodes of acute OM.

To treat fluid buildup, a small incision can be made into the tympanic membrane, commonly known the ear drum, in a procedure known as a myringotomy. During tympanocentesis, the fluid can be removed with a needle by the surgeon. However, after the incision heals, OM can recur and the fluid can build up again. Thus, tympanostomy tubes, commonly called ear tubes, are used to create a semi-permanent channel for mucus to drain from the middle ear space and allow air to enter, equalizing the pressure and preventing pain. They can also help return the patient's hearing to normal, as the dampening effects of viscous fluid on the ossicles during “glue ear” is no longer present. Grommets (ventilation tubes) for hearing loss associated with otitis media with effusion in children. The lower amount of fluid in the ear can also prevent recurrent OM.

The placement of tympanostomy tubes is frequently recommended for patients with recurrent acute OM, commonly defined as 3 or more episodes of OM within a 6-month period. Tube placement can also be recommended for chronic OM where fluid is present in the middle ear continuously for over 4 months, fluid is causing a documented hearing loss greater than 20 dB, infection does not clear up after trying multiple antibiotics, or complications of ear infections occur including mastoid infection. Nearly 700,000 tympanostomy tube placements are performed each year in the US alone, making it the most common procedure for children under anesthesia. It is estimated that 26% of children require tympanostomy tube insertion before the age of 10. There is increasing prevalence of recurrent otitis media among children in the United States.

To place a tympanostomy tube, a small typically cylindrical grommet is inserted into a small perforation in the tympanic membrane formed during a myringotomy. Tympanostomy tubes are typically composed of silicone or fluoroplastic, although variations have been composed of titanium and stainless steel. They come in a variety of shapes and sizes, and the selection of tube by the surgeon is based on the pathophysiology, the patient's age, the number of previous sets of tubes, the surgeon's preference, and the duration of time for placement. Short-term tubes are smaller and typically stay in place for 2 to 18 months before falling out on their own. Long-term tubes are larger with flanges that secure them in place for up to three years and often require removal by an otolaryngologist.

In addition to being placed directly into a hole in the tympanic membrane, another option is subannular placement via a tunnel beneath the skin of the external ear canal and annulus, which is a bony ring that surrounds the tympanic membrane. This technique can be used for atrophic and retracted tympanic membranes where there can be insufficient fibrous tissue to retain a standard tympanostomy tube. It can also be beneficial for patients who have undergone a tympanoplasty, or a replacement of the tympanic membrane tissue. The materials and designs of subannular ventilation tubes are like those of tympanostomy tubes. For both types of tubes, antibiotic droplets are frequently recommended to allow for local delivery and treatment of recurrent infections.

### BRIEF DESCRIPTION OF THE DRAWINGS

The objects and advantages will be apparent upon consideration of the following detailed description, taken in conjunction with the accompanying drawings, in which like reference characters refer to like parts throughout, and in which:

FIG. 1A illustrates desired features of conduits for controlled fluid transport. FIG. 1B illustrates this concept for an exemplary case of tympanostomy conduits in accordance with certain embodiments. FIG. 1C shows the advantage of using the tympanostomy tubes described in certain embodiments of this disclosure.

FIG. 2 illustrates a tympanostomy conduit according to certain embodiments. FIG. 2 (view a) shows a tympanostomy conduit with occlusion of the lumen and biofilm adhesion to the inner and outer surfaces of the conduit. FIG. 2 (view b) shows a tympanostomy conduit according to certain embodiments with immobilized liquid interfaces on both sides (view I) or one side (view II) of the tube substrate.

FIG. 3A is a schematic illustration of a patterned conduit surface according to certain embodiments. FIG. 3B (view a) shows a photograph of a patterned surface and FIG. 3B (view a) shows tympanostomy conduits featuring grooved surface fabricated by additive manufacturing according to certain embodiments. FIG. 3C (view a) shows a 3D printed silicon sheet without an infused overlayer. FIG. 3C (view b) shows to a silicone sheet with an infused overlayer to improve the smoothness of a 3D printed silicone sheet, according to certain embodiments. FIG. 3D shows a silicone oil wrapping layer around the fluid entering the tube.

FIG. 4A shows the sliding angles of water and mucus on infused and non-infused materials, according to certain embodiments. Sliding angles are measured with the goniometric setup schematically depicted in the inset, according to certain embodiments. FIG. 4B shows sliding angles and contact angle hysteresis of water on infused and non-infused materials, according to certain embodiments.

FIG. 5A shows adhesion of primary human epidermal keratinocytes on infused and non-infused surfaces, according to certain embodiments. FIG. 5B shows adhesion of human neonatal dermal fibroblasts on infused and non-infused surfaces, according to certain embodiments. FIG. 5C shows the maximum adhesion force of HNDFs measured through lateral pull-off using an atomic force microscope.

FIG. 6 shows cytotoxicity of non-infused and oil-infused silicon materials for human epidermal keratinocytes.

FIG. 7A shows adhesion of S. aureus bacteria on infused and non-infused surfaces, according to certain embodiments. FIG. 7B shows adhesion of S. pneumoniae and M catarrhalis bacteria on infused and non-infused surfaces, according to certain embodiments.

FIGS. 8A-8D illustrate bidirectional fluid transport through tympanostomy conduits, in accordance with certain embodiments.

FIG. 9A shows conduit designs in accordance with certain embodiments. FIG. 9A (view a) shows non-infused symmetric tubes, FIG. 9A (view b) shows liquid-infused symmetric tubes and FIG. 9A (view c) shows asymmetric tubes. FIG. 9B demonstrates multipart assembly with functional add-ons/inserts that enable preferential transport of a given liquid in one direction while inhibiting transport of this liquid in the opposite direction, in accordance with certain embodiments.

FIG. 10 illustrates design principles for optimizing the bidirectional flow in the tympanostomy conduit include the size and shape of the flanges, radius and length of the tube lumen, and surface tension of liquids and tube, in accordance with certain embodiments.

FIGS. 11A-11D show a comparison of cylindrical conduits (view a), conical conduits (view b), and curved conduits (view c), in accordance with certain embodiments. FIG. 11A shows a schematic representation of parameters for optimizing the pressure barrier to transport (e.g., initial radius, initial flange angle, and length of the lumen, lubricant) in accordance with certain embodiments. FIG. 11B shows fluid entering the conduit, FIG. 11C shows fluid exiting the tube made of hydrophobic material, and FIG. 11D shows fluid exiting the conduit made of hydrophilic material, in accordance with certain embodiments.

FIG. 12A shows the reduced pressure of aqueous antibiotic drops flowing through optimized conduits of various radii compared to cylindrical and conical conduits. FIG. 12B shows an exemplary optimized curved tube geometry with its length constrained to 2 mm, according to certain embodiments. An exemplary inner distal radius was selected to be 0.275 mm.

FIG. 13 compares Young-Laplace pressures for water and aqueous antibiotics flowing through various tube geometries in accordance with certain embodiments: curved tubes (view a) conical (view b) and cylindrical (view c).

FIG. 14A shows the simulated Young-Laplace pressure along the length of tubes of various geometries in accordance with certain embodiments: curved (views a and d), conical (views b and e), or cylindrical/collar button (views c and f). Views a-c show pressure for aqueous antibiotics. Views d-f show pressure for water. FIG. 14B shows the simulated Young-Laplace pressure along the length of tubes of various geometries, in accordance with certain embodiments: curved (views a and d), conical (views b and e), or cylindrical or collar button (views c and f). In this case, the radius of the tube entrance was selected to be same for all. Views a-c show pressure for antibiotics. Views d-f show pressure for water. FIG. 14C shows the dependence of the ratio of maximum pressures of water and antibiotic drops (selectivity) in various conduits (curved, conical and cylindrical) on the radius of the conduit.

FIG. 15A (views a1-a6) are a schematic illustration of injection molding manufacturing of tubes with cylindrical shape, in accordance with certain embodiments. FIG. 15A (view b) is a schematic of molded cylindrical tubes, and FIG. 15 A (views c1 and c2) are computerized tomography images of molded cylindrical tubes. FIG. 15B (view a) is a schematic illustration of injection molding manufacturing of tubes with curved optimized shape, according to certain embodiments. FIG. 15B (views b1 and b2) show a curved mold according to certain embodiments, and FIG. 15B (views c1 and c2) shows computerized tomography images of molded, curved tubes according to certain embodiments.

FIG. 16 (view a) is a schematic illustration of the experimental setup for measuring water breakthrough pressure in the conduit, in accordance with certain embodiments. FIG. 16 (view b) is a photograph of two setups running different liquids in parallel, according to certain embodiments.

FIG. 17 shows a comparison of the Young-Laplace pressure of aqueous antibiotic drops passing through the medical-grade silicone non-infused and oil-infused (100 cP) Collar Button tubes (ID=0, 51 mm, dark gray and black bars) and curved optimized tubes (ID=0.55 mm, patterned bars), according to certain embodiments.

FIG. 18 Shows an “hourglass” shaped conduit formed by two curved sections.

FIG. 19 shows chemically patterned tympanostomy conduits, in accordance with certain embodiments.

FIG. 20 shows a conduit with a gradient wettability pattern enabled by a composition of hydrophobic and hydrophilic materials, in accordance with certain embodiments.

FIG. 21A is a schematic illustration of conduits with dual chemically- and geometrically-patterned channels for guided transport of liquids through the tube, in accordance with certain embodiments. FIG. 21B shows conduits with multiple chemically- and geometrically-patterned channels, in accordance with certain embodiments. FIG. 21C shows conduits with porous lumens, in accordance with certain embodiments.

FIG. 22 is a schematic illustration of gravity-assisted delivery of the antibiotic drops into the middle ear, in accordance with certain embodiments.

FIGS. 23A-23C show conduits with pinning sites, in accordance with some embodiments. FIG. 23A shows pinning through modulation of the lumen shape. FIG. 23B shows pinning through modulation of the surface. FIG. 23C shows pinning via a cage-shaped handle on top of the conduit or inside the lumen that reduce and/or prevent environmental fluids from entering the conduit, in accordance with certain embodiments.

FIG. 24A is a schematic illustration of a method to minimize invasiveness during the myringotomy, in accordance with certain embodiments, where the conduits size is reduced prior to insertion, and the conduit swells after insertion. FIG. 24B illustrates the swelling kinetics of a medical grade silicone (MED 4960D, radial dimension) upon swelling at 85° C. in medical grade silicone oil with various viscosities, in accordance with certain embodiments.

FIG. 25A shows compression of a silicone conduit in accordance with certain embodiments under applied load. FIG. 25B shows the compression integrity of the “test” tube is demonstrated along two axes, along the lumen (view a) and across the lumen (view b) for the control Baxter Beveled tube silicone with conical geometry, and non-infused and infused curved conduits with same dimensions according to certain embodiments. FIG. 25C shows the elasticity and fatigue resistance of the silicone tympanostomy tubes in accordance with certain embodiments along two axes.

FIG. 26 (view a) shows exemplary mechanical deformation of the cylindrical tube in accordance with certain embodiments during the swelling process, as calculated by the Finite Element Analysis (FEA) model using the commercial ABAQUS/Standard software. FIG. 26 (view b) shows exemplary mechanical deformation of the curved tube in accordance with certain embodiments during the swelling process.

FIG. 27 shows a conduit with a reduced size prior to insertion that swells after insertion to minimize invasiveness during the myringotomy, in accordance with certain embodiments.

FIG. 28 is a schematic illustration of several examples of shape-changing tympanostomy conduits whose flanges can either expand in size (view a), expand in size and change shape (view b), spread apart (view c), or change shape into an architecture that allows for fluid transport through a funneling architecture or other guided flow design (view d), in accordance with certain embodiments.

FIG. 29 Shows a simulation of an embodiment of a shape-changing behavior of a tympanostomy conduit with a bilayer architecture comprising layers with different cross-linking density.

FIGS. 30A-30B show transformable flanges, in accordance with certain embodiments. FIG. 30A shows transformable flanges that expand to sandwich both sides of the tympanic membrane upon expansion. FIG. 30B shows transformable flanges that lock onto the middle ear cavity of in place upon expansion.

FIG. 31 shows a stent-like design of a conduit that expands to form a larger architecture upon shape change, in accordance with certain embodiments.

FIG. 32 shows handles and flanges composed of a material different from the tube's material for a facile insertion of ear tubes, in accordance with certain embodiments.

FIG. 33 shows a dual injection system with a tip with a non-infused small tube and an oil reservoir, in accordance with certain embodiments.

FIG. 34 shows a tympanostomy conduit with flange stiffness matching the section of the tympanic membrane in which it is being placed, in accordance with certain embodiments.

FIGS. 35A-35B show tympanostomy conduits with sensing components, in accordance with certain embodiments. FIG. 35A shows a tube with a tunable printed antenna for sensing temperature, pH and pressure changes, in accordance with certain embodiments. FIG. 35B shows a tube with a built-in sensor for monitoring changes in the middle ear, in accordance with certain embodiments.

FIG. 36 shows a tympanostomy conduit that changes color upon exposure to certain stimuli, in accordance with certain embodiments.

FIG. 37 shows a tympanostomy conduit capable of molecular detection, capture, and release of relevant biomarkers, in accordance with certain embodiments.

FIG. 38A shows a dynamic, programmable conduit which can be actuated on demand through an external stimulus, in accordance with certain embodiments. FIGS. 38B-38C show examples of activation pathways for the programmable conduits, in accordance with certain embodiments;

FIG. 39 shows a wide-flange conduit architecture with a vascular network indicated by black strips in the tube for prolonged drug delivery directly onto the tympanic membrane, in accordance with certain embodiments.

FIG. 40 shows a conduit for transtympanic drug delivery to the round window membrane (view a) through an array of microneedles (view b) in accordance with certain embodiments.

FIG. 41 shows an expandable reservoir on the middle ear side of the tube, in accordance with certain embodiments.

FIG. 42 shows chemically-actuated designs of tympanostomy conduit for targeted lumen opening, in accordance with certain embodiments.

FIG. 43 shows photo-actuated designs of tympanostomy conduit for targeted lumen opening, in accordance with certain embodiments.

FIG. 44 shows gas-permeable gating designs of tympanostomy conduits for targeted lumen opening, in accordance with certain embodiments.

FIGS. 45A-C show solutions for controlled extrusion of the conduit, in accordance with certain embodiments. FIG. 45A shows shape change of the flanges, FIG. 45B shows shape transformation of the outer surface of the conduit, and FIG. 45C shows actuators that expand or collapse, or undergo another type of size/shape and/or chemical transformation, in accordance with certain embodiments.

FIG. 46 shows the endoscopic images acquired during the myringotomy with tympanostomy tube insertion procedure in accordance with certain embodiments in Chinchila lanigera for control Summit Medical Collar Button tube (view a) and oil-infused silicone Collar Button tube (view b) with same dimensions (ID=1.27 mm).

FIG. 47 shows the auditory brain response and distorted product otoacoustic emissions of animals with tympanostomy tubs.

FIG. 48 shows bacterial adhesion to infused and non-infused tympanostomy tubes.

### SUMMARY

In certain embodiments, the present disclosure is directed to providing guidelines for design of medical and fluidic conduits for medical and biological applications, microfluidic devices, membranes, nozzles, bioreactors, transport of coolant and other chemicals through machinery, drainage of waste products from reactions, sensors, food and beverage industry, cosmetics and perfumes, and other applications.

Certain embodiments of the present disclosure describes ventilation or tympanostomy tubes that reduce and/or prevent occlusion by various biofluids, debris, and cells and bacteria.

Certain embodiments of the present disclosure describe tubes that reduce and/or prevent growth of human cells on the outer surface of the tube and the flanges that would prevent early extrusion.

Certain embodiments of the present disclosure describes surfaces that reduce/or prevent the formation of biofilms on their surface to prevent the development of infection in general or otorrhea in the case of ear tubes.

Certain embodiments of the present disclosure recognize that ideal ventilation or ear tubes would be composed of materials with low advancing contact angles and optimized shapes with chosen antibiotic liquid suspensions as to not prevent these from entering the tubes. As described more fully below, this could be accomplished by either altering the material of the tubes, altering the shape of the tubes, and/or altering the composition of the therapeutic droplets themselves to include more surfactants or using oil-based droplets, in accordance with certain embodiments.

Certain embodiments of the present disclosure describe tube designs that allows water to be passively repelled or to actively induce swelling inside of the tube to close it prior to swimming or bathing to improve patient comfort and encourage ear tube use, including during summer months.

Certain embodiments of the present disclosure describe drops of various materials that can be used to temporarily change the shape or fluidic properties of the tube.

Certain embodiments of the present disclosure describe creation of ventilation tubes that can be easily inserted into smaller perforations through dynamic flanges or that include size changing abilities that would alleviate these issues and potentially make it easier for the surgeon to insert the tympanostomy or subannular ventilation tubes.

According to some embodiments, a system includes a device having a conduit having a proximal end, the proximal end having a proximal end radius, a distal end opposite the proximal end, the distal end having a distal end radius, an inner surface connecting the proximal end and the distal end, the inner surface forming a proximal angle at the proximal end and a distal angle at the distal end, the inner surface having surface properties, and an outer surface connecting the proximal end and the distal end; the distal end radius, the proximal end radius, the distal angle, the proximal angle, and the surface properties of the inner surface are selected to: allow entry of a first material to the distal end of the conduit, allow transport of the first material through the conduit along the inner surface toward the proximal end, and allow exit of the first material from the proximal end of the conduit, and resist entry of a second material into the proximal end of the conduit; and the Young-Laplace pressure for the first material is less than Young-Laplace pressure for the second material.

In some embodiments, the difference between the Young-Laplace pressure of the first material and the Young-Laplace pressure of the second material is in the range of 1 and 1,000 Pa.

In some embodiments, a selectivity of the conduit is between 1 and 10, the selectivity being a normalized pressure difference between the Young-Laplace pressure of the first material and the Young-Laplace Pressure of the second material.

In some embodiments, the at least one of an angle or a surface property of the inner surface vary to maintain a substantially constant or reducing Young-Laplace pressure of the first material from the distal end to the proximal end.

In some embodiments, at least one of an angle or a surface property of the inner surface varies such that there is substantially no pinning of the first material from the distal end.

In some embodiments, at least one of an angle or a surface property of the inner surface varies to maintain a Young-Laplace pressure of the first material from the distal end to the proximal end that varies by 10% or less.

In some embodiments, an advancing angle of the first material at the distal end as the first material enters the distal end is less than 90°.

In some embodiments, an advancing angle of the second material at the proximal end is as the second material enters the proximal end is greater than 90°.

In some embodiments, the proximal angle is increased to decrease the breakthrough pressure of the first material at the proximal end.

In some embodiments, the inner diameter of the conduit is 3 mm or less.

In some embodiments, the conduit is a tympanostomy or aeration tube.

In some embodiments, the shape of the conduit is selected from a group consisting of cylindrical, conical, and curved.

In some embodiments, the diameter of the proximal end is greater than the diameter of the distal end.

In some embodiments, the conduit includes a distal flange disposed on the distal end of the conduit.

In some embodiments, the conduit includes a proximal flange disposed on the proximal end of the conduit.

In some embodiments, the device is a tympanostomy tube and at least one of the proximal flange and the distal flange has a radial stiffness that matches a portion of a tympanic membrane.

In some embodiments, the device further includes a portion of the conduit provided with a slippery surface including: a partially or fully stabilized lubricating liquid layer on at least a portion of the inner surface or the outer surface of the conduit, the lubricating liquid layer wetting and adhering to at least a portion of the conduit to form the slippery surface over the portion of the conduit.

In some embodiments, the lubricating liquid decreases an advancing angle of the first material.

In some embodiments, the lubricating liquid increases an advancing angle of the second material.

In some embodiments, the spreading coefficient of the first material on the lubricating liquid is greater than zero, and wherein the lubricating liquid forms a wrapping layer around the first material.

In some embodiments, the lubricating liquid decreases the effective surface tension of the first material.

In some embodiments, the lubricating liquid increases the effective surface tension of the second material.

In some embodiments, the lubricating liquid is on the inner surface of the conduit.

In some embodiments, the lubricating liquid is on the outer surface of the conduit.

In some embodiments, the lubricating liquid is on the inner surface of at least one of the proximal flange and the distal flange.

In some embodiments, the lubricating liquid is one or more of silicone oil, partially or fully fluorinated oil, mineral oil, carbon-based oil, castor oil, fluocinolone acetonide oil, food-grade oil, water, surfactant/surfactant solution, organic solvent, perfluorinated hydrocarbons, as well as mixtures thereof.

In some embodiments, the surface properties include a chemical gradient or pattern on at least a portion of at least one of the inner surface and the outer surface.

In some embodiments, the chemical gradient or pattern is disposed on the inner surface of the conduit.

In some embodiments, the chemical gradient or pattern is disposed on the outer surface of the conduit.

In some embodiments, the chemical gradient or pattern is disposed on at least one of the proximal flange at the proximal end of the conduit and a distal flange at the distal end of the conduit.

In some embodiments, the chemical gradient or pattern decreases the effective surface tension of the first material when the first material is disposed on the chemical gradient.

In some embodiments, the chemical gradient or pattern increases the effective surface tension of the second material when the second material is disposed on the chemical gradient.

In some embodiments, the chemical gradient or pattern includes a wicking layer to configured to transport fluid along the wicking layer from one of the proximal end and the distal end to the other of the proximal end and the distal end or a center portion of the conduit.

In some embodiments, a portion of the conduit is provided with a gradient or pattern thereon.

In some embodiments, the gradient or pattern decreases the effective surface tension of the first material.

In some embodiments, the gradient or pattern increases the effective surface tension of the second material.

In some embodiments, the gradient or pattern is disposed on at least a portion of the inner surface of the conduit.

In some embodiments, the gradient or pattern is disposed on at least a portion of the outer surface of the conduit.

In some embodiments, the gradient or pattern is disposed on at least one of the proximal flange and the distal flange at the distal end of the conduit.

In some embodiments, the gradient or pattern is selected from a group consisting of geometrically patterned channels, macro-porous channels, micro-porous channels, three-dimensional periodic networks of pores, sponge-like networks of pores, surface roughness, grooves, ridges, indentations, micropillars, and microridges.

In some embodiments, the conduit includes a stimulus-responsive portion, the stimulus being selected from one or more of light, temperature, pressure, electric field, magnetic field, swelling, de-swelling, or chemical composition.

In some embodiments, the stimuli-responsive portion is selected from a group consisting of a thermostrictive, piezoelectric, electroactive, chemostrictive, magnetostrictive, photostrictive, swellable, or pH-sensitive material.

In some embodiments, the stimulus is the chemical composition, and the chemical composition includes a lubricating liquid.

In some embodiments, the stimulus-responsive portion includes a proximal flange disposed at or near the proximal end of the conduit; and wherein the distal flange is capable of transitioning between a first configuration and a second configuration in response to the stimulus.

In some embodiments, the distal flange changes at least one of a size of the distal flange or a shape of the distal flange when transitioning between the first configuration and the second configuration.

In some embodiments, one of the distal end and the distal flange includes a protrusion, the protrusion includes a shape constant material to facilitate insertion of the distal end of the conduit.

In some embodiments, the stimuli responsive portion is a valve disposed within the conduit, the valve being capable of closing in response to the stimulus.

In some embodiments, the valve is selected from one of a stimuli-responsive polymer, a gas-selective mobile membrane, stimuli-responsive cilia-like and hair-like fibers, platelets, pillars, reconfigurable tunable nano- or microstructures with functionalized tips, and combinations thereof.

In some embodiments, the stimulus-responsive portion further includes a proximal flange disposed at or near the proximal end of the conduit, and wherein the proximal flange is capable of transitioning between a first configuration and a second configuration in response to the stimulus.

In some embodiments, the stimuli-responsive portion includes a first layer of a first stimuli-responsive material and a second layer of a second stimuli-responsive material,

In some embodiments, the stimulus is swelling and the first stimuli-responsive material and the second stimuli-responsive material have different cross-linking densities.

In some embodiments, the conduit has a first diameter in the first configuration, and the conduit has a second diameter in the second configuration.

In some embodiments, the stimuli-responsive portion is disposed on the inner surface of the conduit.

In some embodiments, the stimuli-responsive portion swells in response to the stimuli.

In some embodiments, the conduit further includes a lumen defined by the inner surface and extending from the distal end to the proximal end, wherein the stimuli-responsive portion is disposed in the lumen.

In some embodiments, the stimuli-responsive portion includes pores disposed throughout the lumen and the pores close in response to the stimulus.

In some embodiments, the lumen is open to the first material in the first configuration and closed to the first material in the second configuration.

In some embodiments, the stimuli-responsive portion is disposed on the outer surface of the conduit.

In some embodiments, the stimulus causes the stimuli-responsive portion to separate from the conduit.

In some embodiments, the stimuli-responsive portion includes actuators that are configured to expand when exposed to the stimulus.

In some embodiments, the conduit includes a tube, and wherein the device further includes a second conduit, the second conduit including a tube having a proximal end and a distal end, the second conduit proximal end disposed near the proximal end of the conduit and the second conduit distal end disposed near the distal end of the conduit.

In some embodiments, the distal end radius, the proximal end radius, the distal angle, the proximal angle, and the surface properties of the inner surface are selected to allow entry of a third material to the proximal end of the conduit, allow transport of the third material through the conduit along the inner surface toward the distal end, and resist exit of the third material from the proximal end of the conduit; wherein the Young-Laplace pressure for the third material is less than the Young-Laplace pressure for the second material, but below the breakthrough pressure at the distal end.

In some embodiments, at least a portion of the inner surface is configured to pin the third material thereon.

In some embodiments, the at least portion includes one of a surface chemistry or a texture to facilitate pinning of the third material.

In some embodiments, the conduit further includes a valve configured to resist exit of the third material from the proximal end of the conduit.

In some embodiments, the difference between the Laplace pressure of the second material and the Laplace pressure of the third material is between 1 Pa and 1000 Pa.

In some embodiments, the distal end is configured to have breakthrough pressure of at least 1 Pa higher than the Young-Laplace pressure of the third material at the location of the distal end to prevent exit of the third material from the distal end.

In some embodiments, the advancing angle of the third material at the proximal end as the third material enters the proximal end is less than 90°.

In some embodiments, the angle of the inner surface at the distal end is decreased to increase the breakthrough pressure of the third material at the proximal end.

In some embodiments, the distal end radius, the proximal end radius, the distal angle, the proximal angle, and the surface properties of the inner surface are selected to allow entry of a fourth material to the proximal end of the conduit, allow transport of the fourth material through the conduit along the inner surface toward the distal end, and allow exit of the first material from the distal end of the conduit; and wherein the Young-Laplace pressure for the fourth material is less than the Yong-Laplace pressure for the second material.

In some embodiments, the difference between the Young-Laplace pressure of the second material and the Laplace pressure of the fourth material is in the range of 1 Pa to 1000 Pa.

In some embodiments, at least one of an angle or a surface property of the inner surface vary to maintain a substantially constant or reducing Young-Laplace pressure of the fourth material from the proximal end to the distal end.

In some embodiments, at least one of an angle or a surface property of the inner surface varies such that there is substantially no pinning of the first material from the proximal end to the distal end.

In some embodiments, an advancing angle of the fourth material at the proximal end as the fourth material enters the proximal end is less than 90°.

In some embodiments, the distal end is configured to have breakthrough pressure for the fourth material of at least 1 Pa lower than the Young-Laplace pressure of the forth liquid at the location of the distal end to enable its exit.

In some embodiments, the angle of the inner surface at the proximal end is increased to decrease the breakthrough pressure of the fourth material at the proximal end.

In some embodiments, the first material is selected from the group consisting of effusion, pus, blood, plasma, tears, breast milk, amniotic fluid, serum, synovial fluid, cerebrospinal fluid, urine, saliva, sputum, sweat, other bodily fluid, water, water containing surfactants, perilymph, endolymph, mucus, and any combination thereof.

In some embodiments, the second material is selected from the group consisting of water, aqueous solutions, foams and emulsions, ototoxic agents, soap, pool water, fresh water, salt-containing water, or precipitation, foams and emulsions, ototoxic agents.

In some embodiments, the third material is selected from a group consisting of lubricating liquids, cross-linkers, aqueous and oil-based solutions of antibiotics, antiseptics, anti-viral agents, anti-inflammatory agents, small molecules, immunologics, nanoparticles, genetic therapies including viral and lipid-based therapies, chemotherapeutics, stem cells, cellular therapeutics, growth factors, proteins, radioactive materials, other liquid or gas-based pharmaceutical compounds, and combinations thereof, cerumenolytic agents, e.g. squalene, chlorhexidine, and EDTA, deferoxamine, dihydroxybenzoic acid, glutathione, D methionine and N acetylcysteine, also in forms of foams and emulsions.

In some embodiments, the fourth material is selected from the group consisting of oil-based, water-based, and other solvent-based therapeutics containing at least one of antibiotics, antiseptics, anti-viral agents, anti-inflammatory agents, small molecules, immunologics, nanoparticles, air for ventilation, genetic therapies including viral and lipid based therapies, chemotherapeutics, stem cells, cellular therapeutics, growth factors, proteins, radioactive materials, other liquid or gas-based pharmaceutical compounds, and combinations thereof.

In some embodiments, the conduit includes one or more of a hydrogel, a chemically crosslinked polymer, a supramolecular polymer, a metal, a metal oxide, a porous material, geometrically-patterned pores or channels in a material, membranes and sponges, colloid- and surfactant-templated pores, grooves and ridges, periodic and aperiodic arrays of indentations, nano- and microstructures: nanoforest, nanoscale patterned films, microplatelets, micropillars, and microridges.

In some embodiments, the conduit includes one or more of biostable or bioabsorbable polymers, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, and chitins.

In some embodiments, the conduit includes one or more of dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, gelatin, collagen, or derivatized versions thereof.

In some embodiments, wherein the conduit includes one or more of Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Ti, Pb, Bi, La, Ce, Pr, Nd, Sm, Eu, Gd, Tb, Dy, Ho, Er, Tm, Yb, Lu, and their oxides.

According to some embodiments, a system includes tympanostomy or ventilation device having a conduit configured to be positioned in an ear, the conduit including an input port configured to be received in an ear canal, the input port configured to receive a first liquid; an output port configured to be received in a middle ear, the output port configured to output the first liquid received in the input port; an inner surface extending from the input port to the output port, at least a portion of the inner surface being a conical or curved geometry extending at least partially between the input port and the output port to allow the transport of the first liquid between the ports.

In some embodiments, the first liquid is a therapeutic.

In some embodiments, the conical or curved geometry is selected to allow the first liquid to pass from the input port to the output port and to prevent a second liquid to pass from the input port to the output port.

In some embodiments, the second liquid is selected from at least one of water, aqueous solutions, foams and emulsions, ototoxic agents, soap, pool water, fresh water, salt-containing water, or precipitation, foams and emulsions, ototoxic agents, and combinations thereof.

In some embodiments, a lubricating liquid layer is disposed on at least part of the inner surface, the lubricating liquid layer including a lubricating liquid that wets and adheres to the at least part of the inner surface to form a slippery surface over the at least part of the inner surface.

In some embodiments, the lubricating liquid and the conical or curved geometry are selected to allow the first liquid to pass from the input port to the output port and to prevent a second liquid to pass from the input port to the output port.

In some embodiments, a pattern is on at least part of the inner surface.

In some embodiments, the pattern includes a wicking layer, the wicking layer is configured to transport fluid along the wicking layer.

In some embodiments, the pattern includes a difference in surface properties of the at least part of the inner surface.

In some embodiments, the surface properties of the at least part of the inner surface change from being hydrophobic at the input port to less hydrophobic or hydrophilic at the output port.

In some embodiments, the pattern is selected from a group consisting of geometrically patterned channels, macro-porous channels, micro-porous channels, three-dimensional periodic networks of pores, sponge-like networks of pores, surface roughness, grooves, ridges, indentations, micropillars, and microridges.

In some embodiments, the lubricating liquid, the pattern, and the curve are selected to allow the first liquid to pass from the input port to the output port and to prevent a second liquid to pass from the input port to the output port.

In some embodiments, the lubricating liquid layer reduces the adhesion of microbes and cells.

In some embodiments, otitis media, puss, mucus can enter the output port in the middle ear, be transported through the tube and exit at the inner port into the ear canal.

In some embodiments, at least one of the input port further includes an input port flange configured to assist entrance of the first material into the input port, and the output port further comprise an output port flange configured to assist the entrance of a third material into the output port.

In some embodiments, the third material is selected from the group consisting of effusion, pus, blood, plasma, tears, breast milk, amniotic fluid, serum, synovial fluid, cerebrospinal fluid, urine, saliva, sputum, sweat, other bodily fluid, water, water containing surfactants, perilymph, endolymph, mucus, and any combination thereof.

In some embodiments, the conduit includes a shape, the shape being configured to change in response to a stimulus.

In some embodiments, the shape change is selected from one of closing of the input port, closing of the output port, closing of the inner surface between the input port or output port, and combinations thereof.

In some embodiments, the shape change includes one of increasing the size of the output port, increasing the size of the input port, increasing the size of the conduit, expanding of a flange at the input port, expanding of a flange at the output port, actuation of actuators on an external surface of the conduit, or combinations thereof.

In some embodiments, the shape change includes one of decreasing the size of the output port, decreasing the size of the input port, decreasing the size of the conduit, contracting of a flange at the input port, contracting of a flange at the output port, actuation of external actuators on the conduit, or combinations thereof.

Upon review of the description and embodiments provided herein, those skilled in the art will understand that modifications and equivalent substitutions can be performed in carrying out the invention without departing from the essence of the invention. Thus, the invention is not meant to be limiting by the embodiments described below.

### DETAILED DESCRIPTION

I. Problems with Tympanostomy Tubes and Conduits

Problems with tubes, such as tympanostomy and subannular ventilation tubes, are common.

For example, to place a tympanostomy tube, a small typically cylindrical grommet is inserted into a small perforation in the tympanic membrane. Tympanostomy tubes can be composed of silicone or fluoroplastic, although variations have been composed of titanium and stainless steel. They come in a variety of shapes and sizes, and the selection of tube by the surgeon is based on the pathophysiology, the patient's age, the number of previous sets of tubes, the surgeon's preference, and the duration of time for placement. Short-term tubes are smaller and typically stay in place for 2 to 18 months before falling out on their own. Long-term tubes are larger with flanges that secure them in place for up to three years and often require removal by an otolaryngologist.

In addition to being placed directly into a hole in the tympanic membrane, another option is subannular placement via a tunnel beneath the skin of the external ear canal and annulus, which is a bony ring that surrounds the tympanic membrane. This technique can be used for atrophic and retracted tympanic membranes where there can be insufficient fibrous tissue to retain a standard tympanostomy tube. It can also be beneficial for patients who have undergone a tympanoplasty, or a replacement of the tympanic membrane tissue. The materials and designs of subannular ventilation tubes are like those of tympanostomy tubes. For both types of tubes, antibiotic droplets are frequently recommended to allow for local delivery and treatment of recurrent infections.

A. Occlusion of Tubes

It is estimated that 7% to 37% of implanted tympanostomy tubes fail due to occlusion. Occlusions can be formed by mucus, blood, keratinocytes, earwax, or bacteria and they prevent fluid from flowing through the tubes, rendering them ineffective. Many tube materials, including silicone and fluoroplastics, although having a low degree of wettability, do not resist adhesion of cells and require high sliding angles for water and mucus droplets to slide from the surfaces. When a tube becomes clogged, ear drops can be prescribed to help loosen the blockage. When possible, the ENT doctor can try to suction out the blockage. Sometimes the patient must undergo a painful procedure to remove the occluded tube. In addition to causing additional medical expenses and increased risk of scarring, tube replacement requires additional surgeon and patient time.

B. Premature Extrusion of Tubes

Keratinocytes are a basal epithelial cell type, forming a layer on the external side of the tympanic membrane. When a tympanostomy tube is placed on or into the tympanic membrane, the squamous layer of the tympanic membrane keratinizes on the outer flange, pushing out the tube posterior-inferiorly and causing extrusion of transtympanic ventilating tubes, relative to the site of insertion. Premature extrusion of tympanostomy tubes can occur, requiring the patient to undergo another tympanostomy tube placement surgery.

C. Failure to Self—Extrude and Medial Migration of the Tube

One of the most serious problems associated with tympanostomy tubes is persistent tympanic membrane perforation. Perforations can need surgical closure via a myringoplasty/tympanoplasty procedure. Higher complication rates, such as persistent otorrhea, formation of granulation tissue, or impending development of cholesteatomas, are observed in patients when tympanostomy tubes stay in the tympanic membrane longer than 2 years. Tympanic membrane perforation has been reported to be more common when the ventilation tube is removed (14.3%) than when it extrudes spontaneously (4.0%). A long-term T-tube with two long flanges usually remain in the eardrum for 24 months or longer and are associated with higher persistent tympanic membrane perforation.

Another rare complication is a medial migration of tympanostomy, in which the tube is displaced behind an intact tympanic membrane instead of following the natural path of extrusion towards the ear canal. Some hypotheses connect this complication with the formation of the biofilm on the outer surface of the tube, and with the dysfunction of the Eustachian tube.

D. Biofilm Formation on Tubes

Ventilation tubes can serve as a site for bacterial adhesion and biofilm formation. Bacterial biofilms are glycoprotein bacterial colonies that are resistant to antibiotic penetration. In addition to clogging, this can cause additional infections within the middle ear space. Otorrhea is the most common postoperative complication of middle ear ventilation tube insertion. Ootorrhea can form because of a biofilm in the middle ear, serving as a bacterial reservoir for bacteria to be continuously released into the middle ear. Postoperative otorrhea requires antibiotics and aggressive treatment, and often requires the tube to be removed because of permanent contamination of the tube. Thus, bacterial adherence to tympanostomy tube materials has been the focus of study for more than 30 years. In vitro studies have demonstrated that more inert tympanostomy tube materials and smoother surfaces can inhibit the adsorption of key bacterial binding proteins, such as fibronectin. Biofilms will form on each type of tympanostomy tube currently available on the market.

E. Delivery of Therapeutic Droplets Through Tubes

To prevent the negative side effects of systemic antibiotic usage, targeted therapeutic delivery to the site of infection would be ideal to solve recurrent OM. However, traversing the keratinized tissue of the tympanic membrane on its own to reach the middle ear space is impossible for most droplet formulations. Thus, ventilation tubes can be used to directly deliver antibiotic droplets into the middle ear. However, delivery of single droplets through these small orifices can be challenging. The current materials and geometric space for these tubes, including metals and various plastics, have not been able to solve these issues as the advancing contact angle of these materials with water and other fluids creates an extremely high-pressure resisting entrance of the droplet into the tube. Researchers found that without the use of slight tragal pressure, Cortisporin, TobraDex, and Cipro drops did not consistently pass through tympanostomy tubes.

Currently, for disorders like idiopathic sudden sensorineural hearing loss, clinicians will inject (via a needle) steroids into the middle ear that will ideally diffuse through the round or oval window into the inner ear. While there is an option to place a tube and apply steroid-based ear drops, most clinicians intuitively understand that based on current tube design and flow mechanics, the steroid concentration of drug will not consistently or reliably be high enough to treat the hearing loss. The creation of the tube that allows high flow will allow minimally invasive drug delivery and development of optimized formulations of topical medications, in accordance with certain embodiments.

F. Environmental Water Entering the Middle Ear Space

Environmental water encountered during swimming and bathing, particularly soapy water containing surfactants, can enter the middle ear space, causing pain and additional infections.

G. Invasive Insertion and Scarring

Many tympanostomy tubes require relatively large incisions due to their bulky flanges and surgical placement through the narrow and long ear canal. These large incisions can cause scarring, called tympanosclerosis, and incomplete perforation healing in approximately 5% of cases. Small perforations do not allow sound to be adequately captured and conducted, and scar tissue on the tympanic membrane causes it to be thicker and dampens the motion.

H. Reduced Fluid Flow Through Small Radius Tubes

Movement of fluids through small tubes such as tympanostomy tubes can be challenging. The advancing contact angle of tube materials and water other fluids contributes to an extremely high pressure that prevents fluid from entering and flowing along the length of tubes. Although tubes with small radii are desirable, the high pressures encountered create a lower limit for tube diameter. In addition, high pressures limit the utility of tympanostomy tubes for drug delivery to the middle ear.

### II. Design Principles of Conduits for Controlled Fluid Transport

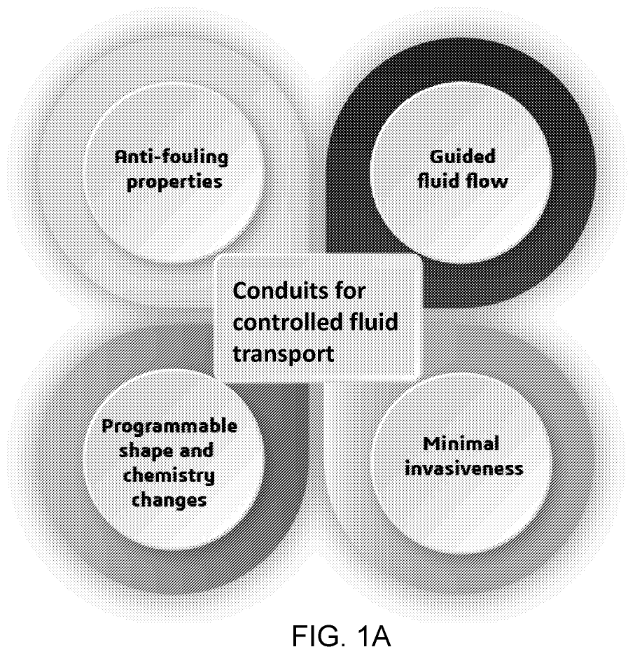

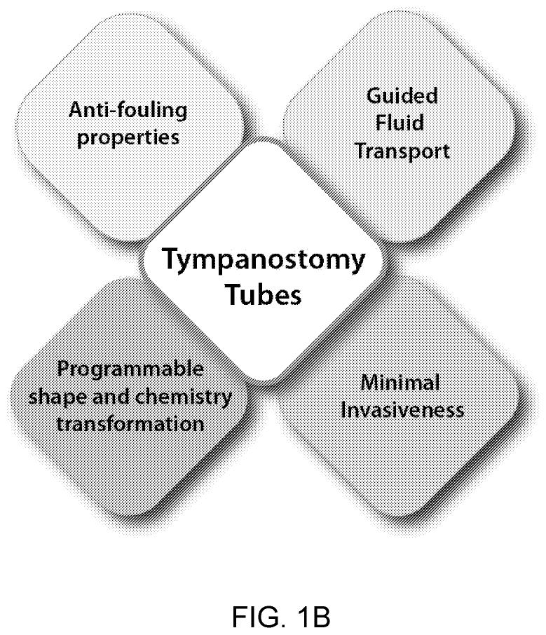

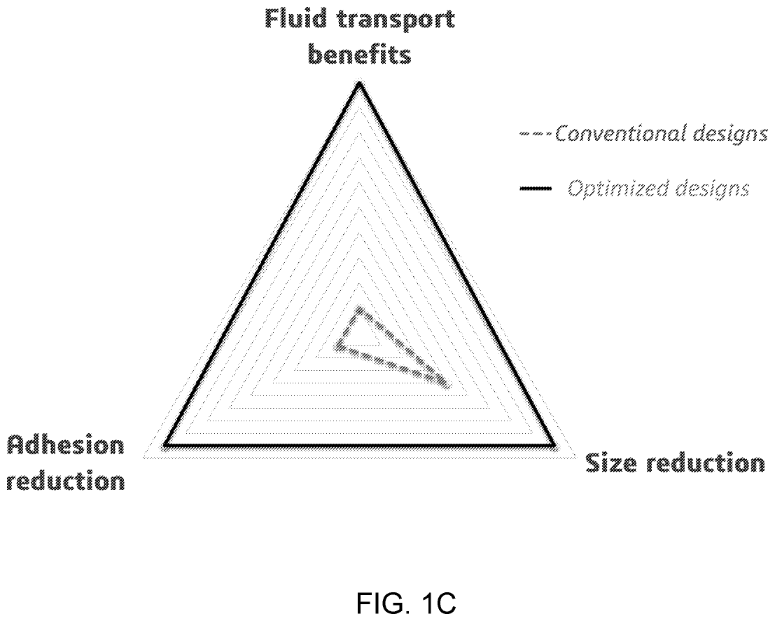

In accordance with certain embodiments, disclosed herein are improved conduits for various application. In accordance with certain embodiments, disclosed herein are tympanostomy and/or subannular ventilation conduits. The geometry and/or surface properties of these tubes or conduits are optimized for controlled transport of various fluids. These conduits can be provided with any desired shape such as flat, curved, wavy, round, tubular, cylindrical, conical, sharpened, beveled, isotropic and anisotropic, mesh-like, membrane-like, catheter-like, flower-like, wire-like. The conduits can be all smooth or roughened, solid or porous, mono- or multilayered, soft or hard, hollow or filled with one or more additional functional materials or therapeutics. The conduits can include fully- or partially biodegradable parts. The conduits can have chemically or structurally patterned surfaces. The conduits can have one or more soft or hard flanges. The conduit can have one or more of the properties described in FIG. 1: A) anti-fouling properties, B) guided fluid transport, C) minimal invasiveness, and D) programmable “on-demand” shape and chemistry transformation.

Some of the exemplary design principles discussed in the present application include the reduction and/or prevention of occlusion on the lumen of the conduit, reduction of adhesion of the biofilm to the inner and outer surfaces of the conduit, enhanced guided flow of biological fluids and antibiotic drops, reduction and/or prevention of an early extrusion of the conduit, smoothing of the inner and outer surfaces of the tube by adding the lubricious or lubricating layer, inducing a wrapping layer on the biological fluids, antibiotic drops, cells and bacteria, on-demand replenishment of the lubricating overlayer, minimization of invasiveness, avoiding hearing loss and formation of the scarring tissue in the tympanic membrane, patient-specific customization of tube, patient-specific customization of drug, on-demand change of geometry and surface chemistry of the tube, controlled capture and release of biomarkers in the middle and outer ear, patterning of the tube to improve the fluid transport and bioadhesion, and remote monitoring of the middle ear condition through built-in sensors.

While certain embodiments of the present disclosure discuss tympanostomy conduits, and others discuss subannular ventilation conduits, it shall be understood that the tympanostomy conduit designs and principles herein can be used for subannular ventilation conduits, and the subannular ventilation conduits designs herein can be used for tympanostomy conduits. Additionally, the conduit designs herein can be used for other medical and biological purposes outside of the middle ear. Non-limiting examples include inner ear conduits, prostatic and biliary stents, sinus cavities, stents for sinus cavities, abdominally-based drains, such as drainage of gallbladder, pancrease, intestine.

Other non-limiting examples include eye tubes, such as glaucoma shunts or tear duct tubes. According to study by Worth Health Organization in 2002, glaucoma is the second leading cause of blindness. Glaucoma patients requiring surgical treatments often use glaucoma drainage devices such as Ahmed Glaucoma Valve (AGV), Baerveldt, or Molteno. Glaucoma drainage devices are designed to divert aqueous humor (fluid in the eye) from the anterior chamber to an external reservoir. Glaucoma drainage devices devices allow to control intraocular pressure (IOP) in eyes with previously failed trabeculectomy and in eyes with insufficient conjunctiva because of scarring from prior surgical procedures or injuries. Glaucoma drainage devices devices are available in different sizes, materials, and design with the presence or absence of an TOP regulating valve, yet they often face many postoperative complications such as hypotony due to a poor drainage regulation, occlusion, corneal scarring, and others. All these complications require more surgeries and treatment which can lead to unforeseen complications, and inoperable patients; while untreated postoperative hypotony can lead to blindness. Hence the move to minimizing repeated surgeries by improving the fluid flow regulation is a constant goal of certain embodiments.

In certain embodiments the conduits address the problem of tear duct clogging. Tear duct clogging occurs due to the obstruction of tear drainage system and can cause responses such as infection, swelling, allergic reaction, tumor, or injury. Tear duct clogging affects up to 5% of infants in United States. Many treatments currently exist to treat tear duct clogs depending on cause and severity. One of treatments includes the insertion of lacrimal stents (or canalicular stents). The two main divisions of stents are bicanalicular versus monocanalicular. Placement of nasolacrimal stents can also sometimes result in an occlusion and infection linked to biofilm production from organisms such as nontuberculous mycobacteria

A particular advantage of embodiments of this invention is that they can reduce the need for revision surgery and can be customized and optimized for a host of various specific clinical indications. The designer tympanostomy conduits discussed in the embodiments of the present disclosure can serve custom patient needs as seen in the Table 1, including important ones such as Eustachian tube dysfunction and sensorineural hearing loss and others, in a minimally-invasive fashion. Either one or two, or the synergy of benefits shown in the FIG. 1C can be useful and be enabled by materials and geomentry considerations disclosed in the embodiments of this invention. Advantages of certain embodiments compared to other conduit material designs include reduction of size, improved fluid transport, and reduction of bacterial and cellular adhesion. A synergy of benefits of tympanostomy conduits can be attained utilizing the material-design combinations of tubes described in this application, in accordance with certain embodiments. In some embodiments, certain benefits can be achieved only through synergistic utilization of several functionalities of the designer tympanostomy tube toolbox shown in the FIG. 1B.

TABLE 1
Designer Tympanostomy Tubes for Specific Clinical Indications in Certain Embodiments

| | | Drug | | Water | Long | On/Off | Anti- | Clog |
|---|---|---|---|---|---|---|---|---|
| Indication | Ventilation | Delivery | Fluid Egress | Precautions | duration | Capacity | microbial | Resistance |
| Chronic | *** | * | *** | *** | *** | * | *** | *** |
| Serous Otitis | | | | | | | | |
| Media | | | | | | | | |
| Recurrent | *** | *** | *** | *** | *** | * | *** | *** |
| Acute Otitis | | | | | | | | |
| Media | | | | | | | | |
| Eustachian | *** | * | * | * | *** | * | * | *** |
| Tube | | | | | | | | |
| Dysfunction | | | | | | | | |
| Sensorineural | * | *** | * | * | ** | ** | * | *** |
| Hearing Loss | | | | | | | | |
| Meniere's | * | *** | * | * | ** | ** | * | ** |
| Disease | | | | | | | | |
| Autoimmune | * | *** | * | * | ** | ** | * | ** |
| Hearing Loss | | | | | | | | |
| Short term | *** | * | * | * | ** | *** | * | ** |
| Ventilation | | | | | | | | |

The number of * indicates greater degree of importance.

In certain embodiments, the surface properties and shape of the tube are selected to meet certain patient needs. For patients with chronic serous otitis media (pediatric and adult), ventilation is a primary issue due to poor Eustachian tube function, thus the tube needs to stay clog-free, and, thus, to have low-adhesion surface and stay in the eardrum for a desired amount of time. Avoiding water is important in pediatric patients, thus selective permeability is of importance. For the recurrent acute otitis media, the ability to administer antibiotic ear drops (drug delivery) is critical, thus tubes can be optimized for flow in both direction: into and out of the middle ear. For Eustachian tube dysfunction in adults, ventilation is a primary issue, as well as the need for long term duration, thus tubes with low-adhesion properties are desired. For patients with inner ear diseases (adults with Sensorineural hearing loss, Meniere's, Autoimmune hearing loss, etc.), primary concern is the drug delivery. For short-term ventilation in adults an On/Off capacity of the tube is a primary concern, e.g. ‘open’ when going on airplane flight and ‘close’ tube when not concerned about barotrauma.

A particular advantage of certain embodiments of the invention is the ability to deliver drugs into infected area.

In certain embodiments a unique feature of dynamic, shape-changing tubes and their uses is described.

Additional advantages of the present embodiments of the invention will become readily apparent to those skilled in this art from the following detailed description, wherein only the preferred embodiment of the invention is shown and described, simply by way of illustration of one of the best mode contemplated of carrying out the invention. As will be realized, the invention is capable of other and different embodiments, and its several details are capable of modifications in various obvious respects, all without departing from the embodiments of the invention. Accordingly, the drawings and description are to be regarded as illustrative in nature, and not as restrictive.

### III. Anti-Fouling Properties

In certain embodiments, medical conduits such as tympanostomy conduits and/or subannular ventilation conduits can be made with anti-fouling materials on the inside of the conduit to reduce and/or prevent occlusion and/or on the outside of the conduit, to reduce and/or prevent premature rejection, minimize the pervasiveness of the infection, and reduce inflammation, improve the smoothness of the tube, and provide a protective coating, e.g. in the form of a wrapping layer, over the impinging biofluid, microorganism, wax and dust. While the following description includes certain embodiments relating to tympanostomy conduits and/or subannular ventilation conduits, the designs can be used in other medical (catheters, inflation balloons, stents, drainages and other) or non-medical applications, such as microfluidic, membrane, bioreactors, transport of coolant and other chemicals through machinery, drainage of waste products from reactions, sensors, printing nozzles, food and beverage industry, cosmetics and perfumes, and other applications.

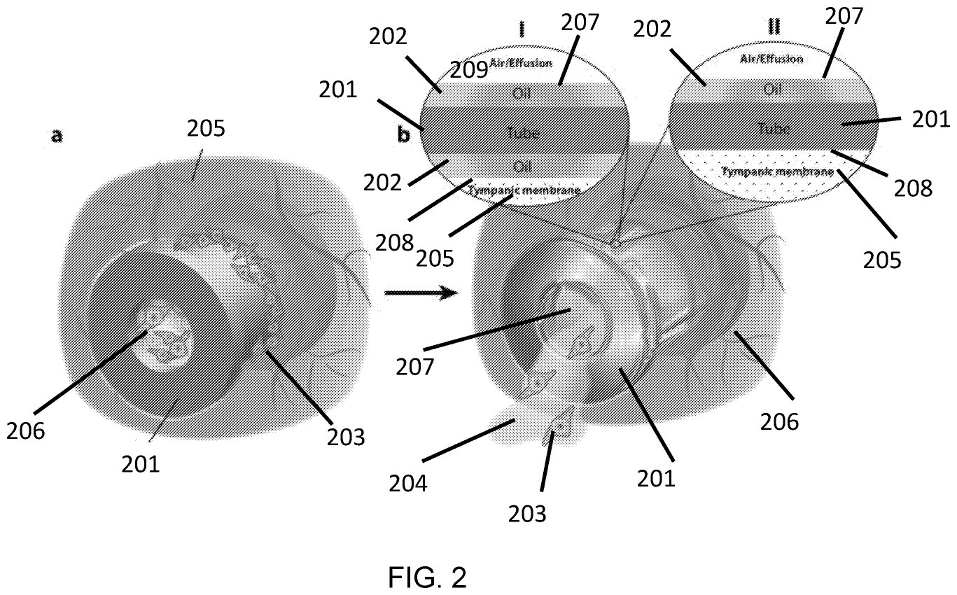

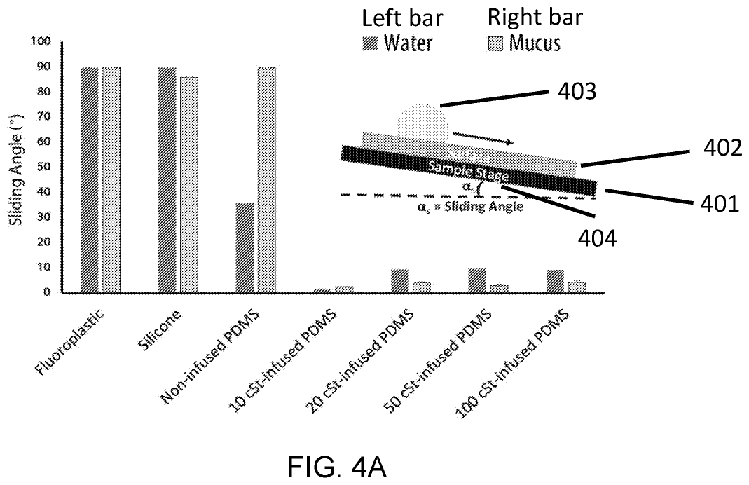

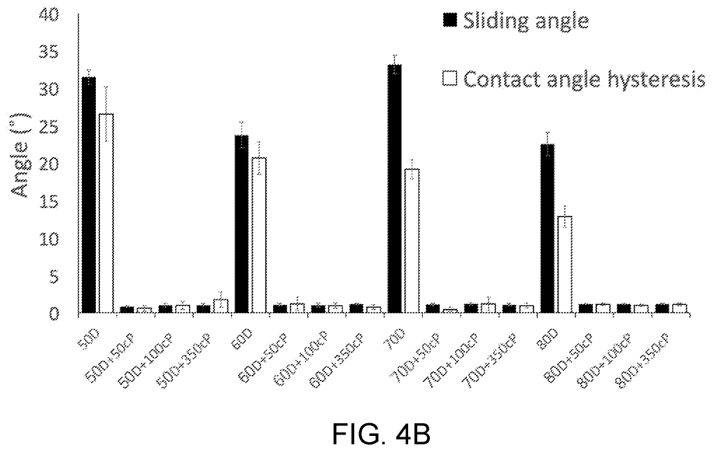

In certain embodiments, a material utilized in designs of tympanostomy tubes 201 makes use of an immobilized liquid interface that can contribute to low cell adhesion and high mobility of liquids on a solid swellable or non-swellable substrate, as shown in FIG. 2 (view b). The stabilized or partially stabilized or temporarily stabilized lubricating liquid layer 202 masks the solid surface of the tubes and creates a slippery self-healing surface and resists or reduces adhesion by cells 203 and immiscible liquids 204 when the tympanostomy tubes are inserted into the tympanic membrane 205 or other physiological membrane (see FIGS. 4-6). The lubricating liquid can be stabilized on the outer surface 207 of the tube, the inner surface 208 of the tube, or on both the inner and outer surfaces of the tube. Lubricating liquid on the inner surface, which faces air or effusion 209, can prevent occlusion of the lumen 206 by preventing adhesion by cells and immiscible liquids. Lubricating liquid on the outer surface of the tube can prevent formation of a biofilm by preventing adhesion by cells and immiscible fluids.

A detailed discussion of the liquid-infused slippery surfaces can be found in U.S. Pat. No. 9,683,197—Issued Jun. 20, 2017, entitled “Dynamic and switchable slippery surfaces”, U.S. Pat. No. 9,121,306—Issued Sep. 1, 2015, entitled “Slippery surfaces with high pressure stability, optical transparency, and self-healing characteristic”, U.S. Pat. No. 9,630,224—Issued Apr. 25, 2017 entitled “Slippery liquid-infused porous surfaces having improved stability”, US Patent Application Publication No. 2015/0152270—Published Jun. 4, 2015, entitled “Slippery self-lubricating polymer surfaces”, US Patent Application Publication No. 2012/021929—Published Jul. 3, 2014, entitled “Slippery Liquid-infused Porous Surfaces and Biological Applications Thereof”, US Patent Application Publication No. 2015/0175814—Published Jun. 25, 2015, entitled “SLIPS Surface Based on Metal-Containing Compound”, US Patent Application Publication No. 20160032074—Published Feb. 4, 2016—entitled “Solidifiable composition for preparation of liquid-infused slippery surfaces and methods of applying”, US Patent Application Publication No. 2014/0342954—Published Nov. 20, 2014, entitled “Modification of surfaces for fluid and solid repellency”, US Patent Application Publication No. 2015/0173883—published Jun. 25, 2015—entitled “Modification of surfaces for simultaneous repellency and targeted binding of desired moieties”, the content of which is hereby incorporated herein by reference in its entirety. In certain embodiments, the lubricating liquid layer above the solid surface can be stabilized fully or partially or temporarily by many different effects, including capillary forces induced by micro/nanoscale topography (10 nm-1000 μm), molecular porosity, surface chemistry, Van der Waals interactions, and combinations thereof. Thus, the underlying solid can be smooth, possess roughness/porosity, and/or be capable of swelling with the lubricating phase. Further, in certain embodiments, the lubricant can be made dynamically stable by liquid flow. In certain embodiments, surfaces with partially stabilized lubricating liquid layers, or lubricating liquid layers that are only stable under flow, also improve performance. In certain embodiments, easily reconfigurable molecules possessing highly flexible long chains with low energy barriers for internal rotation (such as long polydimethylsiloxane polymers or other types of polymers and copolymers, including random or block silicone co-polymers with other siloxane co-monomers featuring alkyl, aryl, aralkyl substituents on silicon atoms) can be grafted to a solid surface and continue to exhibit liquid-like behavior, providing some of the benefits of surfaces with a stabilized lubricating liquid layer.

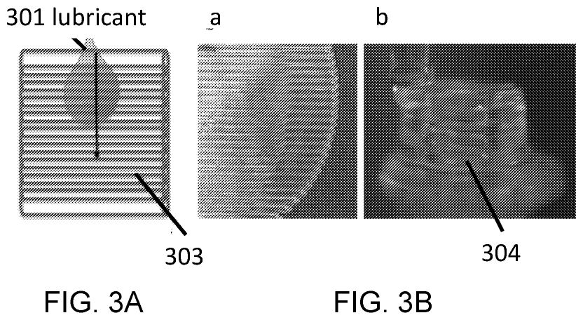

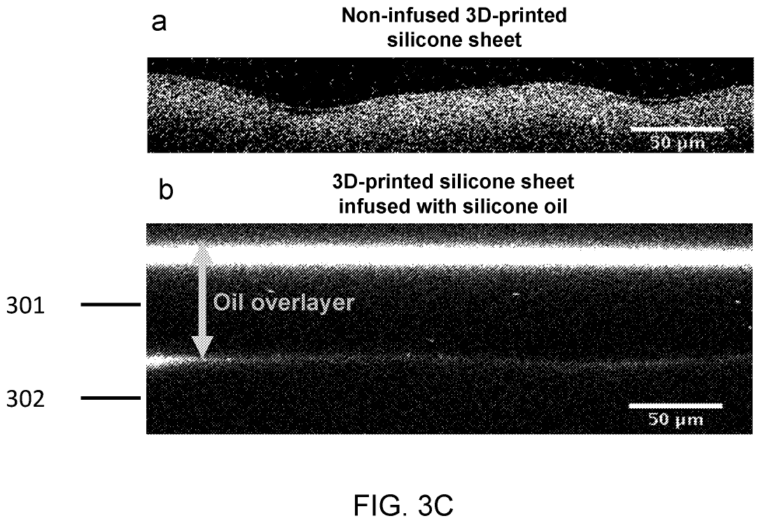

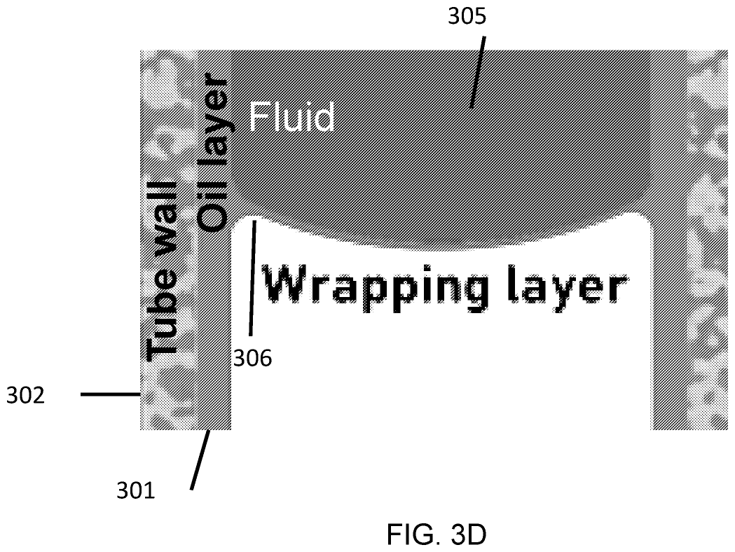

In certain embodiments, shown in FIGS. 3A-3B, conduits can be designed to have texture or patterned morphology (e.g. grooves, pillars and other geometries with the dimensions in the range between 0.01-1 μm or 1-1000 μm or 1000-10000 μm) that help retain the lubricant or lubricating liquid 301 over longer periods of time and during times of large transport of fluid over the surface 302, as depicted in FIGS. 3A-3B. For example, micron-sized grooves can enhance the longevity of the immobilized oil interface by retaining the lubricating liquid. In certain embodiments the lubricating liquid layer 301 fills in the grooves 303 and ridges, with roughness RMS ranging between 10 nm and 1000 μm, thus providing effective smoothening of any adhesion and pinning sites that lead to clogging, biofilm formation and ineffective flow through the conduit 304, as shown in FIG. 3C. The ultra-smooth surface of the lubricating liquid layer 301 is capable of recovering its original shape upon external deformation. As used herein, “ultra-smooth” surface means a surface having a roughness factor that is equal or close to 1, where the roughness factor (R) is defined by the ratio of the real surface area to the projected surface area. Because fluid surfaces generally have a roughness factor of 1, and the top surface in a slippery surface is a lubricating liquid that fully coats the substrate above its hills, surfaces such as a lubricant-coated conduit can be called ultra-smooth. In certain embodiments, ultra-smooth surfaces can have an average surface roughness on the order of or less than about 1 nm. In certain embodiments, “ultra-smooth” can refer to a substantially molecularly or even atomically flat surface. The absence of any defects or roughness on such a surface can aid in minimizing the pinning points for a sliding fluid, thus reducing the contact angle hysteresis, rendering it nearly friction-free and slippery. A detailed discussion of the SLIPS can be found in U.S. Pat. No. 9,932,484, entitled “Slippery Liquid-infused Porous Surfaces and Biological Applications Thereof” filed Jan. 19, 2012, the content of which is hereby incorporated herein by reference in its entirety.

In certain embodiments, as shown in the FIG. 3D, engineering an enhanced wrapping-layer effect of the lubricant 301 around the contacting fluid 305 will allow for facilitated removal of bacteria and cells, wax, mucus and blood, from the surface 302 of the conduit, Advantageously, in some embodiments the wrapping layer 306 can be facilitated by the application of lower-viscosity oil or other lubricating liquid layer onto the surface to enhance the mobility of the impinging biofluid or microorganism on the surface of the fluid, and decrease the rate of post-operative otorrhea as compared to a tympanostomy tube without a lubricating liquid layer. In certain embodiments the lubricating liquid layer will allow for reduced coagulation of blood. The longevity of the lubricated rough surfaces can be engineered by choosing lubricants with low evaporation rate or high viscosity, low miscibility, and reduced wrapping of the lubricant around the contacting fluid. In still further embodiments, the lubricant can be one or more of an oleophobic lubricant, an oleophilic lubricant, a hydrophobic lubricant and/or a hydrophilic lubricant, and/or an omniphobic lubricant. This lubricating liquid layer can allow removal of a large number of bacterial strains including clinical isolates, relevant and not relevant to otitis, S. aureus, H. influenzae, M catarrhalis, S. pneumoniae, and P. aeruginosa, B. catarrhalis, S. epidermidis, and others.

Synergistically with other benefits of the design space of FIG. 1B, one can take advantage of multiple properties, such as change of shape and size. In some embodiments, re-lubrication or the addition of a different lubricant with lower viscosity can increase swelling of the conduit material and thus change of shape and size of the conduit. In certain embodiments, addition of a different lubricant with lower viscosity facilitate the removal of the cellular or biofilm, and trigger a facilitated release of the biofilm from the surface. In addition, a change of geometry towards a more curved one, can eliminate pinning sites caused by angles, improve the fluidic properties, and reduce the adhesion of unwanted surface contamination.

In certain embodiments, further modification of the surface of the tube through adding chemistries (or structures) will improve the benefits of adding the one or more benefit mechanisms from the designer toolbox (FIG. 1B). Those skilled in the art will recognize that there are wide varieties of chemical functionalization agents and methods that would provide the conduits the desired surface chemistry: hydrophobic, hydrophilic, oleophobic, oleophilic, omniphobic. In certain embodiments, the functionalization methodologies can include liquid- or gas-phase reactions or depositions. In certain embodiments, functionalization can involve deposition of primers and top coats, pretreatment with plasma or with reactive chemicals that would render the surface susceptible to further functionalization leading to installation of moieties possessing desired surface energy and ability to attract or repel certain fluids, liquids, complex liquids, heterogeneous emulsions and suspensions, and complex biological matter. Non-limiting examples of hydrophobic moieties are long chain hydrocarbons of linear and/or branched architectures. Non-limiting examples of hydrophilic moieties are polyethyleneglycol chains and their analogs of different molecular architectures. Non-limiting examples of omniphobic moities are polyfluorinated straight and branched (hydro)carbon chains with or without heteroatoms in the chain. Those skilled in the art will recognize that these examples demonstrate a general approach to chemical modification, without limiting to any particular deposition methodology or types of chemical reactions used to functionalize the conduit surface. These examples are non-limiting simply illustrate a variety of approaches that can be used to render the conduit surface attractive or repellant towards the object or medium of interest.

Other non-limiting examples of surface modification include reconfigurable molecules possessing highly flexible long chains with low energy barriers for internal rotation (such as long polydimethylsiloxane polymers or other types of polymers and copolymers, including random or block silicone co-polymers with other siloxane co-monomers featuring alkyl, aryl, aralkyl substituents on silicon atoms) that can be grafted to a solid surface and continue to exhibit liquid-like behavior, providing some of the benefits of surfaces with a stabilized lubricating liquid layer. Other non-limiting examples include lithography, micropatterning, 3D printing, etching, or the plasma treatment, conjugation of proteins or short polymer chains, ionic bonding of small molecules, addition of hydrogen bonded moieties, or infusion of other liquids or gassesand etching.

FIG. 4A depicts a bar graph showing average plus or minus standard deviations of the sliding angles of water (left bar) and mucus (right bar) on different surfaces, including commercial silicone, commercial fluoroplastic (Teflon), non-infused PDMS SE1700 flat sheets, and PDMS SE1700 sheets infused in 10 cSt, 20 cSt and 50 cSt silicone oils, measured with the goniometric setup schematically depicted in the inset. Using the goniometric setup, the surface 401 is placed on a sample stage 402, and a fluid 403 is placed on the surface. The sample stage is tilted until the samples stage meets the sliding angle 404 at which the fluid slides off the surface. FIG. 4B depicts a bar graph showing average sliding angles (±standard deviation) of medical grade silicones infused in medical grade silicone oils 50 cP, 100 cP and 350 cP, as well as their corresponding contact angle hysteresis (difference in the advancing and receding contact angles), in accordance with certain embodiments. A drastic decrease of the sliding angle for oil-infused silicone sheets manifests an application of the immobilized liquid interfaces as anti-fouling coatings for the tympanostomy and subannular conduits according to certain embodiments.

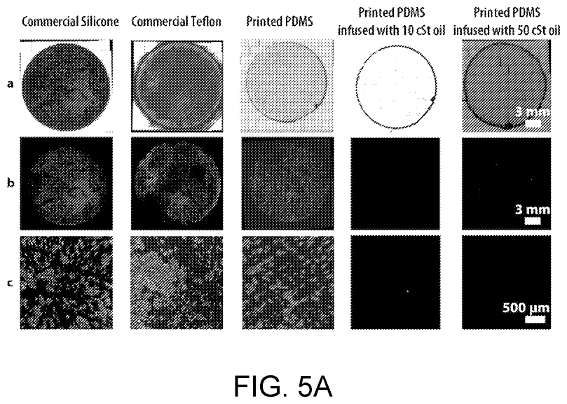

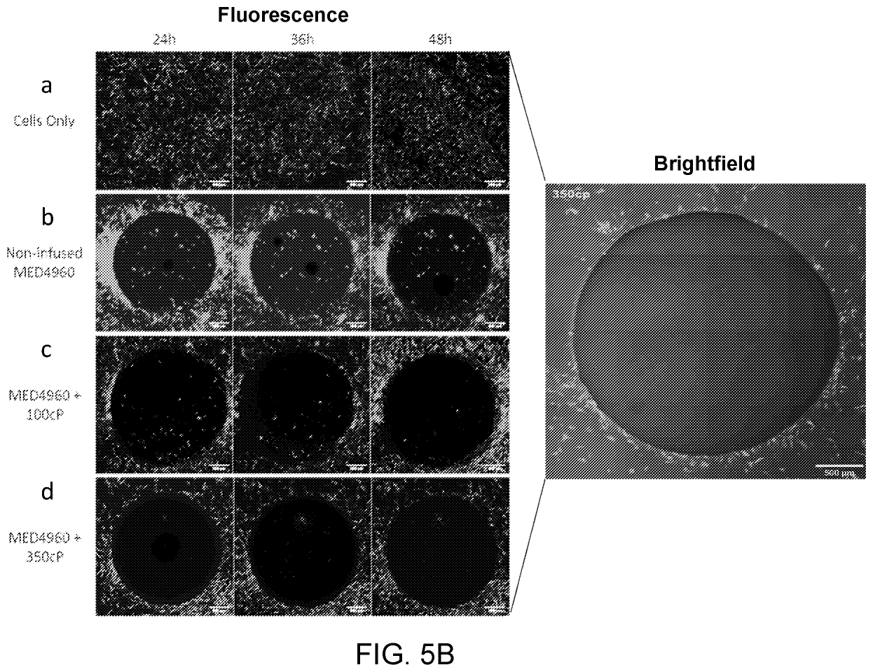

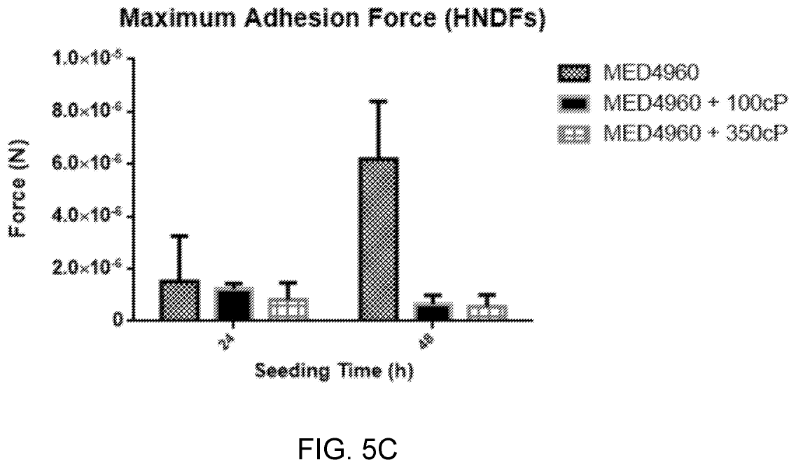

FIG. 5A depicts a comparative study of primary human epidermal keratinocyte adhesion to commercial silicone, commercial fluoroplastic, non-infused PDMS SE1700 flat sheets, and PDMS SE1700 sheets infused in 10 cSt and 50 cSt silicone oils, demonstrating an extremely low adhesion of cells to liquid-infused silicone sheets, as shown in brightfield images (FIG. 5A view a and view b), and fluorescence microscopy images (FIG. 5A view c). FIG. 5A demonstrates an extremely low adhesion of cells to liquid infused silicone sheets. FIG. 5B depicts a comparative study of adhesion of human neonatal dermal fibroblasts (HNDF), modified with enhanced green fluorescent protein (EGFP, λₑₓ=488 nm), to medical grade silicones infused in medical grade silicone oils, and at different time points. For confocal imaging, HNDFs were seeded onto silicone discs for 12, 24, 36, and 48 hours in 6-well plates at a density of 50,000 cells·cm⁻². Cells were then incubated at 37° C. with 5% CO₂ atmosphere until imaging. Cellular adhesion was assessed by quantifying the cell coverage recorded by a confocal scanning laser microscope. Confocal z-stacks across the entire surface area of the samples were taken at 5× magnification and tile-stitched together. FIG. 5B demonstrates low adhesions of HNDFs to silicone-infused silicone sheets. The amount of fluorescence, corresponding to attached cells, is higher on non-infused surfaces (FIG. 5B view b) compared to infused surfaces (FIG. 5B views c and d). Thus, the number of human cells is lower on infused surfaces than non-infused surfaces, implying that these cell lines adhere to these surfaces at a lower amount. Thus, tympanostomy tubes made of these materials likely will have a lower rate of clogging by granulation tissue and/or premature extrusion from the eardrum by a keratinocyte layer growing behind the external flange of the tube.

FIG. 5C depicts a comparative study of HNDF adhesion force to medical grade silicones infused in medical grade silicone oils measured using the lateral pull-off by an atomic force microscope (NanoWizard 4a, JPK Instruments) with silicon AFM probes (All-In-One-Al, BudgetSensors) at 37° C. Cells were pulled laterally from the surface by engaging the tip on one side of the cell and pulling across it with the AFM in constant height mode. The resulting peak deflection was converted to a lateral force. This study also confirms significantly lower adhesion force of HNDFs on oil-infused surfaces according to certain embodiments compared to non-infused surfaces at 48 h after seeding.

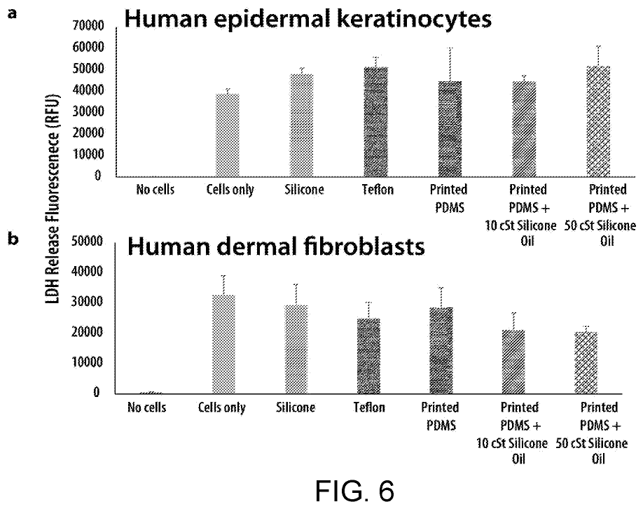

FIG. 6 shows a comparative study of cytotoxicity as quantified by a lactate dehydrogenase (LDH) fluorescence assay for human epidermal keratinocytes (FIG. 6 view a) and human dermal fibroblasts (FIG. 6 view b) cultured on commercial silicone, commercial fluoroplastic, non-infused PDMS SE1700 flat sheets, and PDMS SE1700 sheets infused in 100 cSt and 50 cSt silicone oils, demonstrating a low toxicity of oil-infused PDMS sheets. In certain embodiments, the type of lubricant is chosen based on criteria including longevity, uptake amount into the material, amount of dissipation into surrounding tissue, and amount of cell and biofilm adhesion over time.

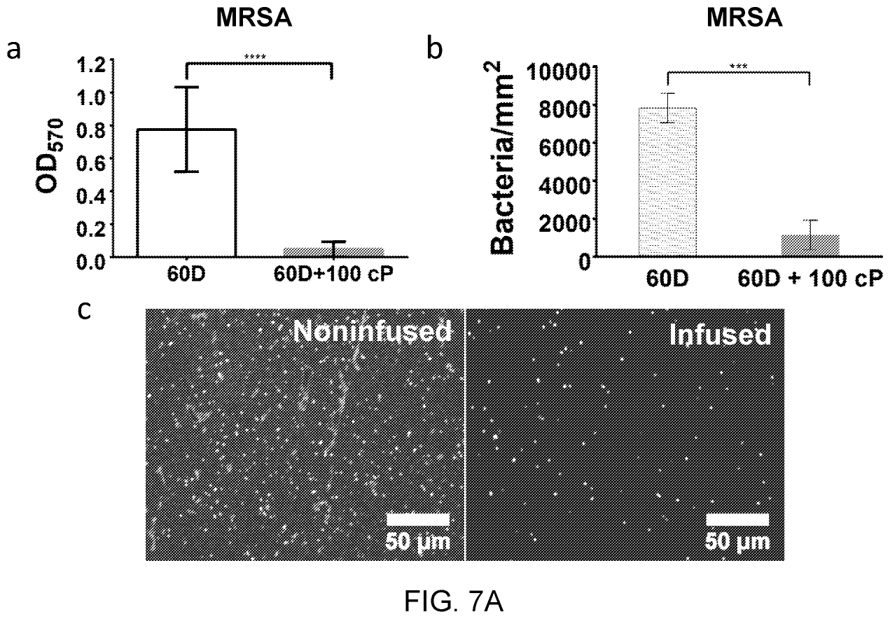

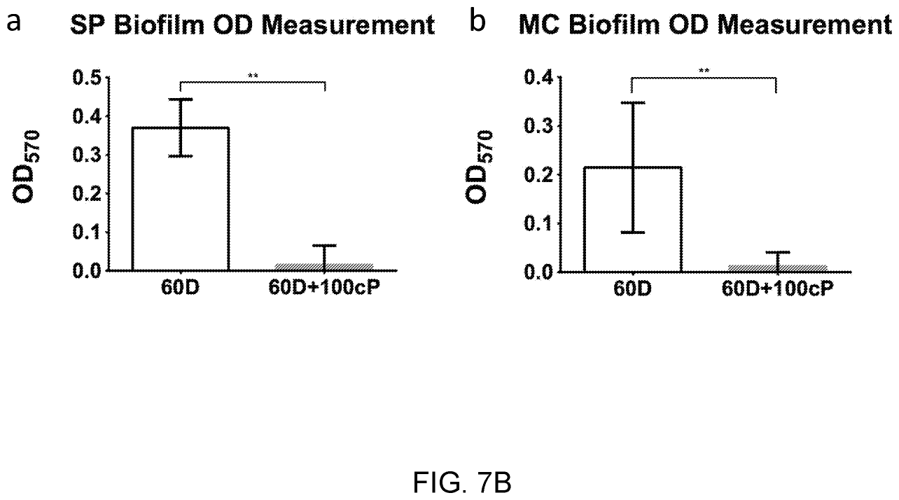

FIG. 7A (view a) depicts a comparative study of adhesion of some exemplary clinical isolates of methicillin-resistant S. aureus (SA), recovered from patients with chronic otitis media seen at the Massachusetts Eye and Ear Infirmary (MEEI), to non-infused medical grade silicones and infused in medical grade silicone oil (100 cP), demonstrating an extremely low adhesion (FIG. 7A views a-b) of bacteria to liquid-infused silicone sheets as shown in fluorescence microscopy images. Samples were then stained with 0.5 w/v % crystal violet for 10 min and rinsed with PBS. The remaining dye that stained the samples was then resuspended with 7% glacial acetic acid. Absorbance (FIG. 7A view a) of the suspended solution was measured at 570 nm. Larger optical density (OD) values correspond to larger quantities of bacteria and biofilm found on the surface of the samples. FIG. 7A (view c) shows confocal microscopy images of bacterial adhesion on a non-infused silicone sample and a silicone sample (MED 4960) infused with medical grade silicone oil (100 cP MED361) after 24 h of immersion in a bacterial broth. The non-infused silicone sample shows a higher density of live bacteria along with the formation of an extracellular matrix. The infused silicone sample shows a lower density of bacteria with no signs of a biofilm matrix.

FIG. 7B (view a) depicts a comparative study of adhesion of some exemplary clinical isolates of M. catarrhalis (MC), S. pneumoniae (SP), recovered from patients with chronic otitis media seen at the Massachusetts Eye and Ear Infirmary (MEEI), to non-infused medical grade silicone and infused in medical grade silicone oil (100 cP). Bacteria exhibit an extremely low adhesion to liquid-infused silicone sheets, as shown in absorbance images (FIG. 7B views a-b). FIG. 7B compares the OD readings of crystal violet staining assays used on non-infused (MED 4960) samples and infused samples (100 cP MED361). The infused silicone sample shows much lower density of bacteria with no signs of a biofilm matrix.

In certain embodiments, other types of anti-fouling coatings can include hydrophobic and hydrophilic materials, some of which are discussed below regarding guided fluid transport.

### IV. Guided Fluid Transport

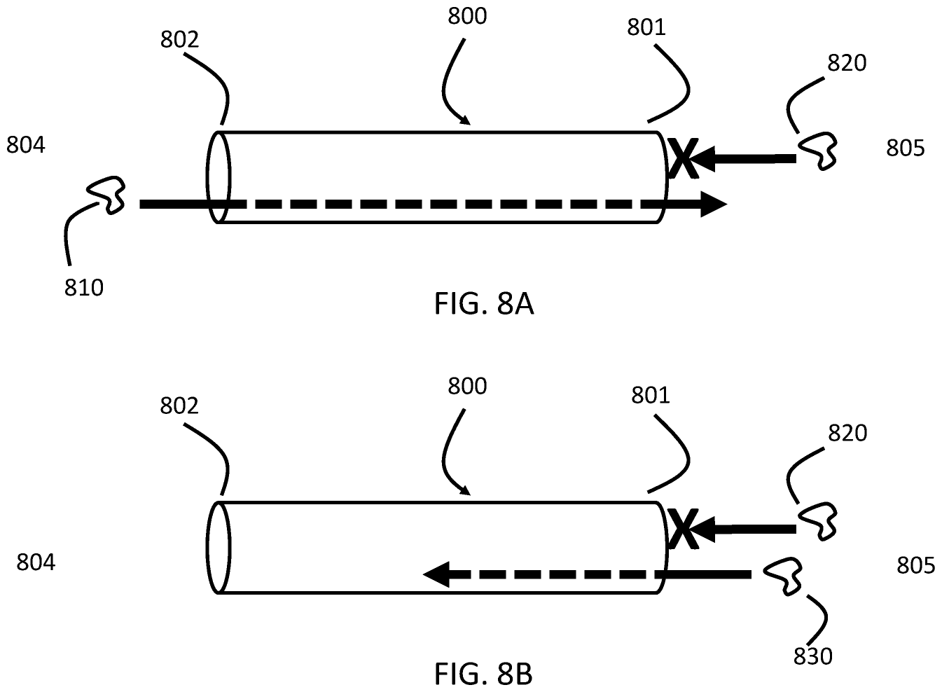

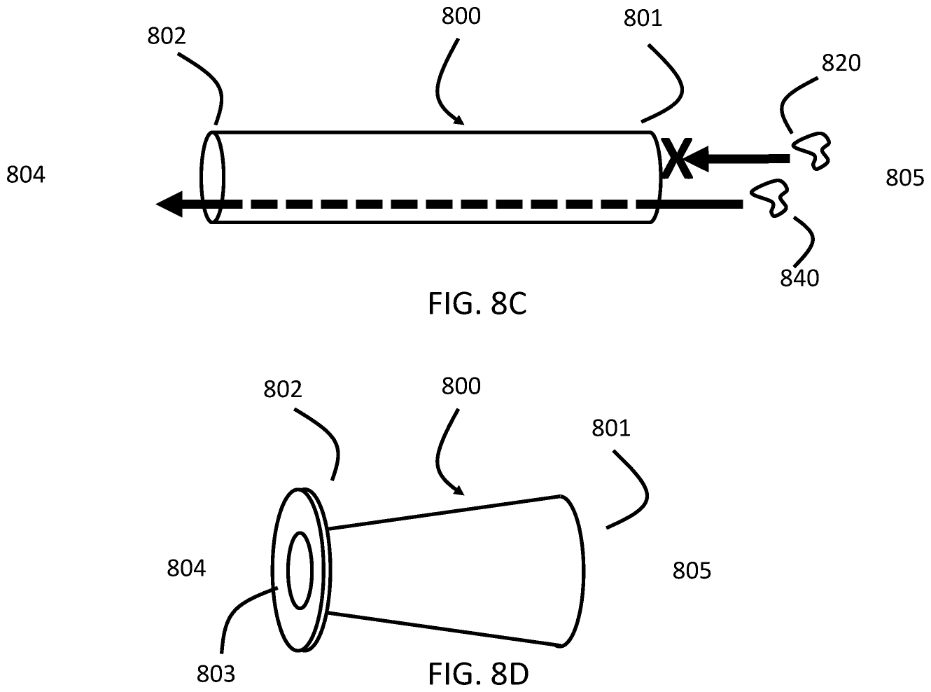

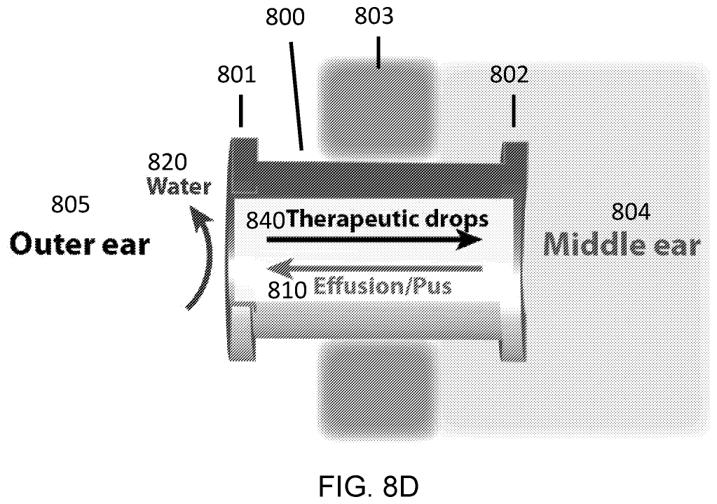

In certain embodiments, directed fluid transport can be designed to occur through conduits, such as tympanostomy conduits, in more than one direction, as shown in FIG. 8A, number of optimized designs can allow for certain fluids to be selectively transported through the conduit while others are or hindered. Although certain embodiments describe selective transport through tympanostomy conduits, it is understood that other embodiments can use conduits in other applications. Where certain embodiments describe a conduit spanning the tympanic membrane, it is understood that the conduit can span other membranes or tissue barriers in the body. Where certain embodiments describe a conduit having a distal end in the middle ear, and a proximal end in the outer ear, it is understood that the conduit can have its distal end in other inner compartments of the body and its proximal end in other outer spaces or compartments.

In certain embodiments, shown in FIG. 8A, the conduit 800 has a distal end 801 or tube entrance and a proximal end or tube exit 801. In embodiments where the conduit is a tympanostomy tube, the tube spans the tympanic membrane 803, the distal end is in the middle ear 804, and the proximal end is in the outer ear 805. In certain embodiments, the distal end radius and the proximal end radius can be selected to control flow of fluid through the conduit. In certain embodiments, flow of fluid through the tube can be controlled by the curvature or angle of the inner surface of the conduit. For example, the inner surface can form a proximal angle at the proximal end and a distal angle at the distal end. In certain embodiments, the surface properties of the inner surface can be selected to control fluid flow. For example, the proximal end or the distal end can have surface properties, such as hydrophobicity or hydrophilicity to control fluid flow in one or both directions.

In certain embodiments, it is desirable for certain fluids to be transported from the distal end to the proximal end. In these embodiments, the distal end is the entrance, and the proximal end is the exit for that material. In other embodiments, it is desirable for other fluids to be transported from the proximal end to the distal end. In these embodiments, the proximal end is the entrance and the distal end is the exit for that material. In certain embodiments, it is desirable for other fluids to be prevented from entering the conduit.

In certain embodiments, the surface properties and shape of the conduit can be controlled such that a first material can exit the middle ear, be transported from the distal end to the proximal end of the conduit without pinning and exit the conduit, but not enter the middle ear, from the proximal end to the distal end of the conduit. In certain embodiments, shown in FIG. 8A, the surface properties and shape of the conduit 800 are selected to allow a first material 810 to enter the distal end 802 of the conduit 800, be transported through the conduit 800 toward the proximal end 801, and exit the proximal end 801 of the conduit 800 more easily than the first material 810 can enter the proximal end 801 of the conduit 800, be transported through the conduit 800 toward the distal end 802, and exit the distal end 802 of the conduit 800. In certain embodiments, the surface properties and shape of the conduit can be controlled so that a second material cannot enter the middle ear. In this embodiment, the surface properties and shape of the conduit 800 are selected to prevent a second material 820 from entering the proximal end 801 of the conduit 800. In this embodiment, it can be desirable to remove a bodily fluid from a compartment of the body, such as the middle ear, and to prevent other fluids from entering this compartment. The first material 810 can be, for example, effusion, pus, blood, perilymph, endolymph, plasma, tears, breast milk, amniotic fluid, serum, synovial fluid, perilymph, endolymph, urine, saliva, sputum, sweat, any other bodily fluid, water, water containing surfactants, mucus, and any combination thereof. The second material 820 can be, for example, water, aqueous solutions, foams and emulsions, ototoxic agents, soap, pool water, fresh water, salt-containing water, or precipitation, foams and emulsions, or ototoxic agents.

In certain embodiments, the surface properties and shape of the conduit can be controlled so that a third material can enter the conduit at the proximal end, but not enter the middle ear. In certain embodiments, shown in FIG. 8B, the surface properties and the shape of the conduit 800 are selected to allow a third material 830 to enter the proximal end 801 of the conduit 800 and be transported through the conduit 800 toward the distal end 802 more easily than the third material 830 can enter the distal end 802 of the conduit 800 and be transported through the conduit 800 toward the proximal end 801 and to prevent the third material 830 from exiting the distal end 802. In this embodiment, it is desirable for the material to enter the conduit 800, for example, to alter the surface properties, shape, or texture of the conduit 800 or replenish a lubricious layer, but it is undesirable for the material to enter a compartment of the body, such as the middle ear. The third material can be, for example, a lubricating liquid, a cross-linker or other chemical composition that acts as a stimulus. In certain embodiments, the third material is a drug that that elutes on the tympanic membrane surface via the tube but not enter the middle ear space.

In certain embodiments, the surface properties and shape of the conduit can be selected so that a fourth material can be delivered to the middle ear by entering the proximal end and exiting the distal end. In certain embodiments, shown in FIG. 8C, the surface properties and the shape of the conduit 800 are selected to allow a fourth material 840 to enter the proximal end 801 of the conduit 800, be transported through the conduit 800 toward the distal end 802, and exit the distal end 802 of the conduit 800 more easily than the fourth material 840 can enter the distal end 802 of the conduit 800, be transported through the conduit 800 toward the proximal end 801, and to exit the proximal end 801 of the conduit 800. It is desirable for the material to enter the conduit 800 and exit into a compartment of the body, such as the middle ear, for example, to deliver a therapeutic. The fourth material 840 can be, for example, oil-based, water-based, or other solvent-based therapeutics containing at least one of aqueous or oil-based solutions of antibiotics, antiseptics, anti-viral agents, anti-inflammatory agents, small molecules, immunologics, nanoparticles, genetic therapies including viral and lipid-based therapies, chemotherapeutics, stem cells, cellular therapeutics, growth factors, proteins, radioactive materials, other liquid or gas-based pharmaceutical compounds, cerumenolytic agents, e.g. squalene, chlorhexidine, and EDTA, deferoxamine, dihydroxybenzoic acid, glutathione, D methionine and N acetylcysteine, also in forms of foams and emulsions, and combinations thereof.

In certain embodiments, shown in FIG. 8D, the conduit 800 has a flange 803 at the distal end 802 of the conduit 800. In other embodiments, the conduit has a flange at the proximal end of the conduit. In some embodiments, the flange 803 is configured to hold the conduit 800 in place in the tympanic membrane. In certain embodiments, the flange is configured to guide fluid. In certain embodiments, the flange is configured to both hold the conduit 800 in place and to guide fluid. In certain embodiments, the flange 803 is flat, angled, or arched.

FIG. 8E shows an exemplary embodiment in which a conduit is secured across tympanic membrane 803 with the distal end 802 at the middle ear 804, and proximal end 801 at the outer ear. According to this exemplary embodiment, the first material 810 discussed above is effusion or puss, the second material 820 discussed above is water, and the third material 840 discussed above is a therapeutic, such as therapeutic drops.

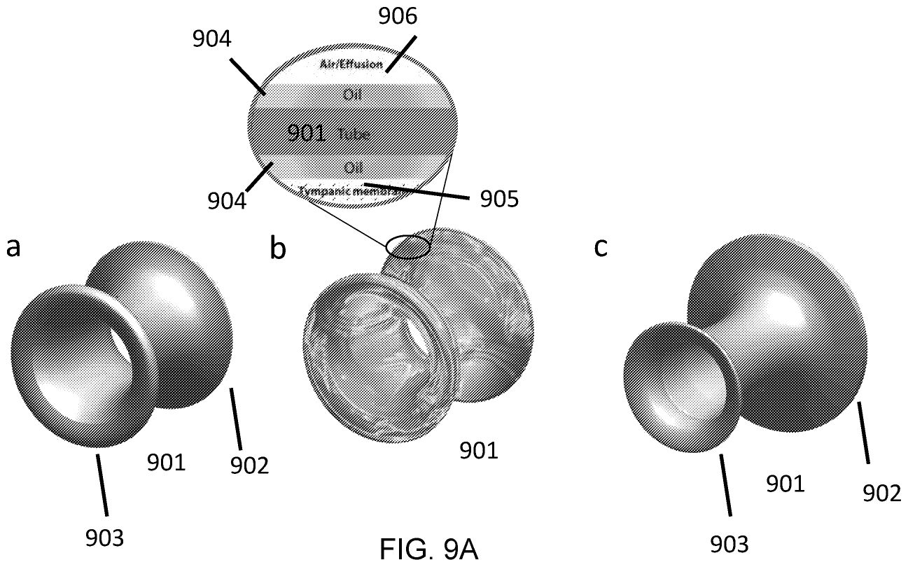

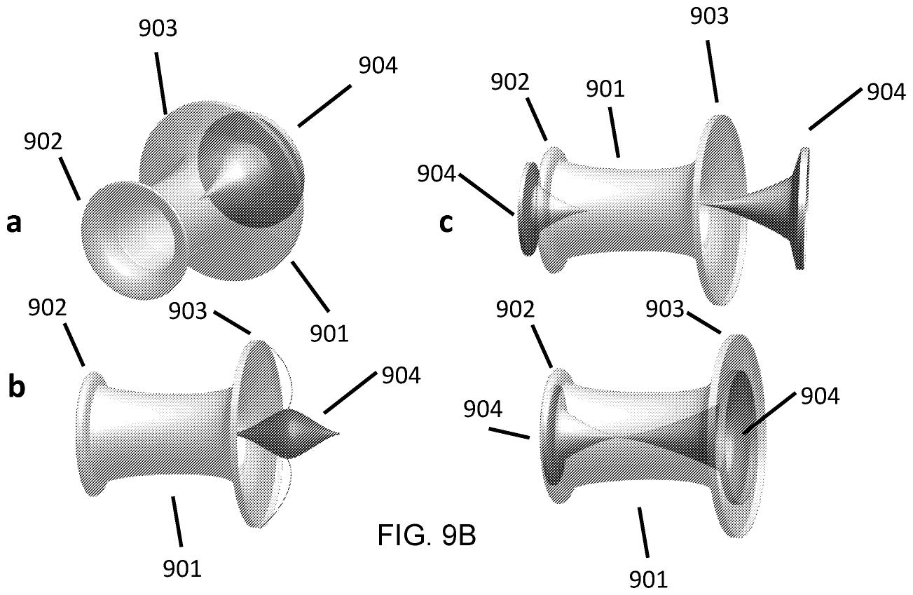

FIG. 9A (view a) shows a symmetric conduit 901 having a distal end 903 and a proximal end 902 with the same diameters, according to certain embodiments. FIG. 9A (view b) shows a symmetric conduit 901 with a lubricating layer (oil) 904 on the inner and outer surfaces of the conduit. In this embodiment, lubricating layer on the outer surface is in contact with the tympanic membrane 905, and the lubricating layer on the inner surface is in contact with air or effusion 906. In certain embodiments, anisotropy in the conduit 901 design can enable preferential transport of a given liquid in one direction while inhibiting transport of this liquid in the opposite direction. Anisotropy can be derived from the macroscopic conduit geometry, as shown, for example, in FIG. 9A (view c). FIG. 9A (view c) shows an asymmetric conduit having a distal end 903 with a first diameter and a proximal end 902 with a second, larger diameter, according to certain embodiments. In this embodiment, a fluid can flow preferentially into the distal end and out of the proximal end. In certain embodiments, anisotropy can be derived from directional micro/nano-topography or porosity, gradient chemical patterning, and/or dynamic features. In certain embodiments, the conduit can have topography or porosity at either the distal or proximal end of the conduit. In other embodiments, the feature sizes of the topography or porosity can be different at the distal end and at the proximal end. In certain embodiments, the conduit can have a chemical or geometric pattern at the distal end or the proximal end. In certain embodiments, the conduit can have a chemical gradient that increases or decreases from the distal end to the proximal end. In certain embodiments, chemical gradients can be installed at the stages of surface functionalization, or conduit fabrication through controlled polymerization, 3D printing, molding and other fabrication methodologies—by exploiting gradients in prepolymer composition, amount and nature of cross-linker, intensity of irradiation, amount of radical initiator and the like. This list of approaches is by no means exhaustive, but rather illustrates the modularity of the designs and tools one can use to achieve the desired transport effects. In other embodiments various regions of the tube surface can carry different chemistries to facilitate anisotropic flow. Non-limiting examples can include differences in hydrophobicity and hydrophilicity, which locally change the liquid contact angles and whether the liquid is pinned or transported through the tube.

In certain embodiments, as shown for examples in FIG. 9B, anisotropy and directional fluid transport can be derived from multipart assembly with functional add-ons/inserts 904. FIG. 9B (views a-c) show certain embodiments of inserts that allow for a droplet placed onto the surface of the insert to spontaneously starts spreading in the direction of a growing insert radius. In certain embodiments, the flow is dominated by capillary forces. At the same time, such anisotropy can allow a different liquid to be transported through the conduit in the opposite direction. FIG. 9B (view a) shows the insert that allows for the flow out of the conduit 901 through the distal end 903. FIG. 9B (view b) shows the insert that allows for the flow both in and out of the conduit through the distal end 903. FIG. 9B (view c) shows two inserts at the proximal end 902 and distal end 903 that allow for the flow in and out of the conduit through the proximal and distal ends.

While the following description includes certain embodiments relating to tympanostomy conduits and/or subannular ventilation conduits, the designs can be used in other medical or non-medical applications, such as microfluidic, membrane, bioreactors, transport of coolant and other chemicals through machinery, drainage of waste products from reactions, sensors, additive manufacturing nozzles, funnels, food and beverage industry, cosmetics and perfumes, and other applications.

In certain embodiments, directionality features designed into the tympanostomy conduits can allow (1) mucus from the middle ear cavity that builds up from otitis media to pass through the conduit into the external auditory canal, and (2) oil- or water-based antibiotic drops delivered through the external auditory canal to pass through these conduits to enter the middle ear cavity, where they can treat the otitis media infection, (3) post-myringotomy blood drainage. A broad range of other liquids can be administered to pass through the conduit in the desired direction. In certain embodiments, directionality features can induce dynamic reversible or irreversible, local or on the whole changes in the tube geometry, surface structure, chemistry or size that can be used for a topical delivery of the drug, drainage of the bodily fluid, improved placement of the device, or structural reconfiguration of the device to aid its stability or extrusion at a desired time.

In some embodiments, the drops administered from one side can temporarily close the tube to temporarily prevent any liquid transport through the conduit. In certain embodiments, drops can block the tympanostomy tube before swimming/bathing to prevent the environmental water from entering the middle ear. In certain embodiments, other stimuli, such as light, temperature, electric or magnetic field, pH change, pressure gradient, and other induce physical or chemical transformation of the tube to serve a desired purpose, in certain embodiments. Exemplary cases are described throughout the disclosure.

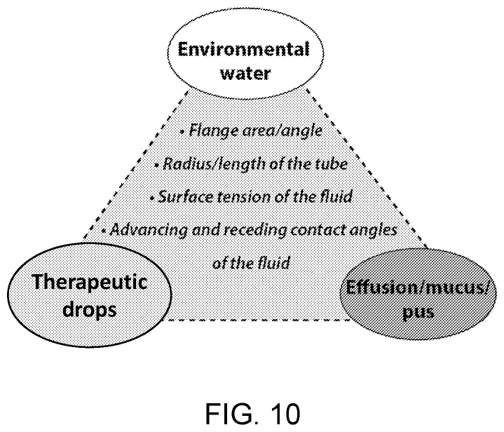

FIG. 10 highlights parameters of the conduits and tested liquids that constitute exemplary design principles of guided enhanced flow through the tympanostomy conduits for environmental water, oil-based ear drops and effusion/mucus/pus in the middle ear, according to certain embodiments. In certain embodiments, the design principles for optimizing the bidirectional flow in the conduit include the size and shape of the flanges, radius and length of the conduit's lumen, curvature of the conduit, chemistry of the surfaces and surface tension of liquids, as well as integration of multiple conduit paths with different properties, each serving a specific directional transport function.

In certain embodiments, geometric patterns can be used for preferential flow. In certain embodiments, the geometric pattern increases the advancing angle and contact angle hysteresis of a liquid entering the conduit, and in other embodiments, the pattern decreases the advancing angle and contact angle hysteresis of a liquid entering the conduit. In certain embodiments, the geometric pattern can induce the Cassie-Baxter. Young-Laplace or Wenzel states, or other intermediate states. In certain embodiments, the geometric pattern is disposed on the outer or inner surface of the conduit. In certain embodiments, the geometric pattern created by surface topography, for example surface roughness, grooves, ridges, indentations, micropillars, microridges, or pores, and other 3D tessellations.

In certain embodiments, various parameters of conduits such as radius, the angle of the flange (the horizontal piece at the end of distal or proximal end) or the lumen wall angle, surface tension, and lubricant can be tuned to either promote fluid flow entering proximal end and exiting distal end or restrict fluid flow in which the fluid is either trapped within the lumen unable to exit the distal end or unable to enter the proximal end.

A. Preventing Fluid from Entering the Proximal End of the Conduit

In certain embodiments, the surface of the conduit can be surface functionalized via chemistries such as but not limited to silanization, fluorination, hydroxylation, carboxylation, and esterification in which the resulting surface is either hydrophobic or hydrophilic. By the use of these surface functionalization, a fluid of hydrophilic or hydrophobic nature can be inhibited from entering at lower Young-Laplace pressures. In certain embodiments, the use of surface-active fluorinated conduit will dramatically increase the Young-Laplace pressure of water entering the conduit compared to a non-polar low surface tension liquid.

In certain embodiments, the radius of the proximal end can be greatly smaller than the distal end to prevent fluid entering the conduit from the proximal end. In certain embodiments, the proximal and distal end are separated by a membrane such as tympanic membrane, anterior chamber, etc. In certain embodiments, this geometry prevents fluid entrance in the proximal end and is preferential minimizing volumetric flow rate.

In certain embodiments, pinning of the liquid can be observed at the proximal end by irregularities in the entrance geometry and cusp within the conduit. The cusp at the entrance of the geometry will induce high Young-Laplace pressures and create potential pinning points.

B. Preventing Fluid from Exiting from the Distal End of the Conduit

In certain embodiments, the angle of the lumen at the distal end can be varied to have a sudden increase in Young-Laplace pressure for fluid exit. For example, in case of Collar Button geometry, the angle of the lumen is maintained as 0° from vertical and hence the sudden change in contact angle the fluid must experience, the fluid must change its contact angle at the distal end from its equilibrium contact angle to the lumen wall to 180°. In this embodiment, the change in angle caused by a discontinuity causes a sudden rise in Young-Laplace pressure for exit. In this embodiment the fluid is therefore within the conduit but barrier is unable to exit due to this sudden pressure.

In certain embodiments, the use of cilia like structures can be used as pinning points within the lumen. Pinning is a phenomenon of discontinuous motion of the meniscus. Pinning is typically induced by discontinuities in the geometry that the meniscus is in contact with, for example through roughness or cilia-like structures. Direction of the structures dictate the preferential direction of flow and hence can be oriented acute to the proximal end preventing fluid from exiting the distal end. Cilia-like structures can be used in combination with radial change through the lumen to prevent the fluid from exiting either end. In certain embodiments, a gradient of surface tension can be imposed on the conduit in which the fluid encounters higher energy barrier as it travels through the conduit reaching the distal end. In certain embodiments, this increase in Gibbs free energy prevents or increases the barrier of the fluid from exiting the distal end. In certain embodiments this can be achieved via gradient of lubricant overlayer thickness, surface tension, density, Young's modulus, or heterogeneity of materials.

C. Conduits Tuned to Induce Optimal Fluid Flow

In certain embodiments, the conduit lumen wall is continuously curved from the proximal end to the distal end to minimize the sudden pressure jump experience by fluid to exit the conduit. In certain embodiments, the summation of the advancing angle and the lumen flange angle at the distal end will be 180° such that the pressure has no discontinuities, hence avoiding any pinning of fluid.

In certain embodiments, the lumen of the conduit is infused with lubricant (or other low surface tension fluid) in which a wrapping layer assist a the flow of fluid through the conduit and out the distal end. In certain embodiments, the wrapping layer allows for the minimization of surface interactions between high surface tension liquid and air. In certain embodiments, a wrapping layer reduces pinning, reducing the Young-Laplace pressure for exit.

In certain embodiments, the conduit is surface functionalized according to the fluid's hydrophilicity or hydrophobicity. In certain embodiments, the fluid is water, and the surface is modified by modifying the lumen wall with metallic elements. In certain embodiments, the fluid preferentially wets the lumen wall without pressure gradients required. In this embodiment, by tuning this surface modification with respect to the fluid to transport, the Young-Laplace pressure of exiting the distal end can be minimized. In other embodiments, the length of the conduit is tuned below the capillary height of the fluid wetting the lumen walls and the radius is below the capillary length of the fluid, allowing the fluid to spontaneously wet and approach the distal end against forces of gravity. In certain embodiments, the fluid is able to flow through the conduit and exit the distal end optimally without the addition of an applied pressure gradient.

D. Tympanostomy Conduits with Optimized Architectures and Surface Chemistry

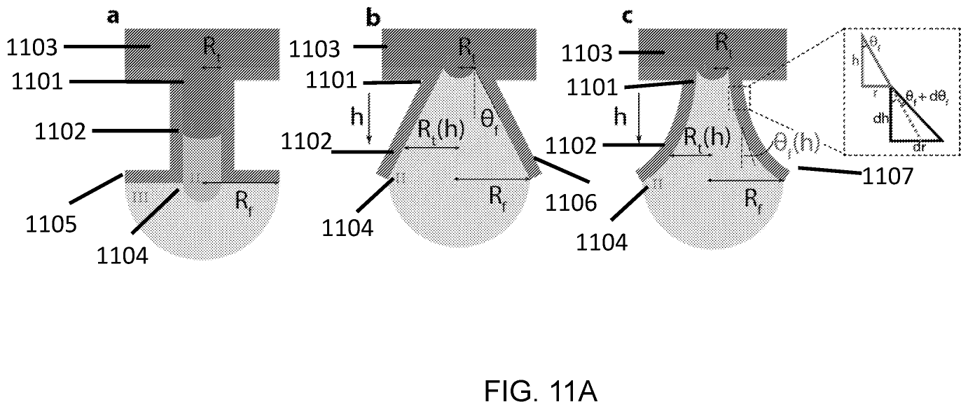

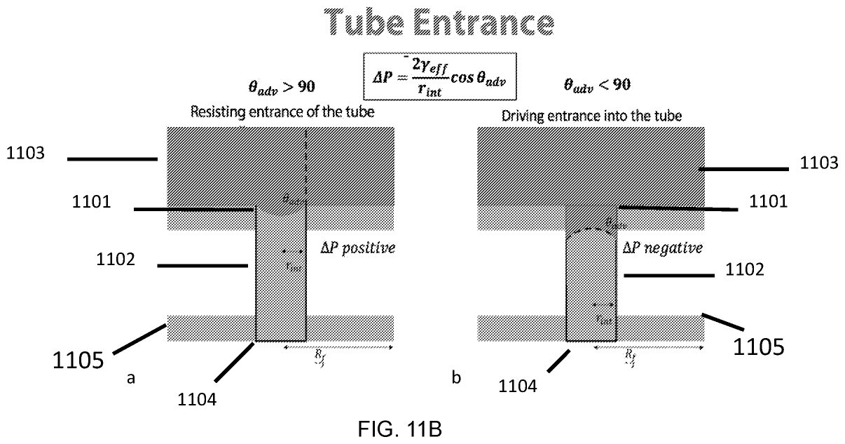

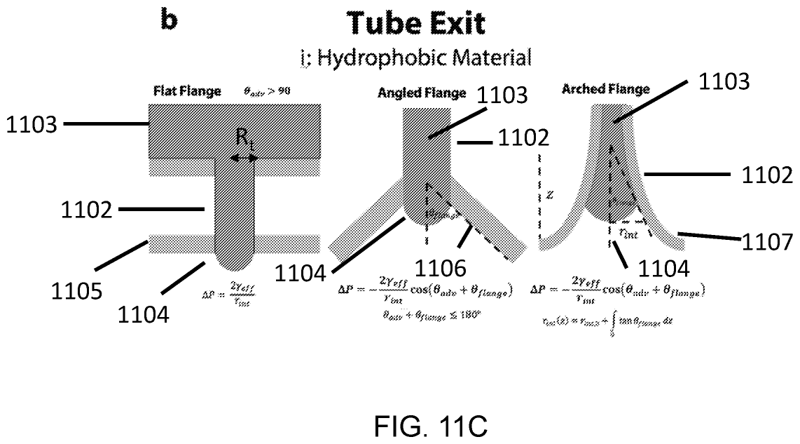

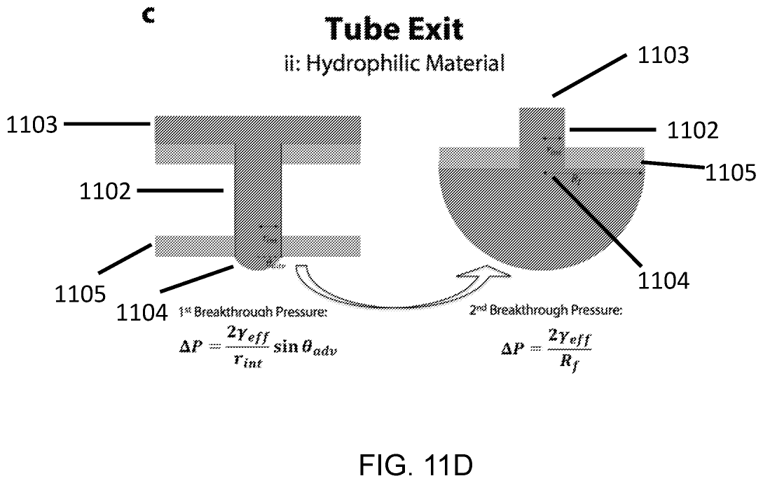

In certain embodiments, to optimize the flow transport through the conduit, to induce or prevent liquid pinning in the tube and thus enable or prevent liquid passage, the shape and surface chemistry of both conduit proximal and distal end is considered. The ability of the liquid to be pinned or be transported inside the tube can most effectively be described in terms of capillary pressure, ΔP, sustained across the interface between two static fluids (e. g. water and air or oil and air, or mucus and air) in the conduit. Pressure can be described by using the Young-Laplace equation:

$$Δ P = - \frac{2 {γ}_{eff}}{{r}_{int}} cos {θ}_{adv} ,$$

where γ_{eff} is the effective surface tension of the liquid entering the conduit, rᵢₙₜ is the inner radius of the conduit, and the θ_{adv} is the advancing contact angle of the liquid, which is a characteristic of the wettability or chemical properties of the surface. Transport through the conduit is constrained by the highest-pressure barrier in the system, which can occur in different areas of the conduit depending on the conduit design (i.e. local geometry and local advancing contact angle along the conduit), direction of liquid transport, shape and curvature of the conduit and flanges, and material properties. When high pressure barriers appear in certain regions, the liquid will pin at these locations and be unable to move within or exit the conduit. In certain embodiments, by keeping the conduit substantially free of significant pressure jumps, as described in non-limiting examples below, liquid pinning can be avoided and transport through the tube can be enabled. As is described below in certain embodiments, such pressure jumps can occur at the entrance or the exit of the tubes, such as when the fluid enters and exits the tube. In certain embodiments the break-through pressure at the conduit ends is optimized and reduced by local changes in chemistry or geometry of the conduit or flanges. A few non-limiting examples are shown in FIGS. 11 A-D. FIG. 11A shows parameters for optimizing the pressure barrier to transport for cylindrical (FIG. 11A view a), conical (FIG. 11A view b), or curved (FIG. 11A view c) conduits, such as initial radius (Rₜ), initial flange angle (Of), length of the lumen (h), and the lubricant utilized. In certain embodiments, by carefully designing the tube geometry and chemistry utilizing the parameters described above, the transport of certain liquids can be facilitated. In certain embodiments, pinning can be induced for other liquids. In other embodiments, anisotropic transport of one liquid in one direction and another liquid in the opposite direction.

1. Surface Properties, Size and Shape of the Conduit and Flanges

In certain embodiments, as can be seen in FIG. 11B, the surface characteristics of the conduit material play a role in driving the liquid into the entrance, or proximal end, 1101 of the conduit 1102. If the advancing angle, θ_{adv}, of the liquid 1103 on the surface at the entrance is less than 90 degrees, then the liquid can be driven into the conduit (FIG. 11B view b), while if θ_{adv}>90 degrees, then there will be a resistance for the liquid to enter (FIG. 11B view a). In various embodiments, slippery surfaces improve transport by decreasing the advancing contact angle by reducing physical pinning and changing the interfacial properties of the material. For lubricant infused surfaces, the contact angle that the liquid makes with the surface can be further reduced by the lubricant physically wrapping over the impinging fluid and reducing its surface energy. The type of lubricating liquid can be adjusted for a custom tube design depending on the application, according to certain embodiments. In certain embodiments, a lubricating layer can also serve as means to elevate the fluid from the surface, so that the flow occurs on a droplet-lubricant interface, instead of a droplet-substrate interface, as dictated by the spreading coefficient theory and minimization for surface free energy.

FIG. 11C shows that in certain embodiments, changing the shape of the exit, or distal end, 1104 of a hydrophobic conduit can contribute to guided fluid transport. In certain embodiments, positioning the flanges at an angle to the hydrophobic conduit allows for a lower differential pressure across the fluid interface. In certain embodiments, the conduit can have a flat flange 1105, an angled flange 1106, or a curved flange 1107. In certain embodiments, the dimension of the exit flange is chosen to decrease the pressure barrier for a droplet leaving the conduit by integrating curvature (see the arched flange in FIG. 11C) such that θ_{flange} is low when rᵢₙₜ is small and increases as rᵢₙₜ grows. In this case,

$$Δ P ( z ) = - \frac{2 {γ}_{eff}}{{r}_{int} ( z )} cos ( {θ}_{adv} + {θ}_{flange} ( z ) ) ,$$

where rᵢₙₜ(z)=r_{int,0}+∫₀^{z} tan θ_{flange} dz and the precise shape is numerically optimized to minimize the maximum breakthrough pressure of a given liquid by ensuring

$$\frac{Δ P ( z )}{dz} = 0.$$

In certain embodiments, for a flat flange,

$$Δ P ( z ) = \frac{2 {γ}_{eff}}{{r}_{int}} ,$$

and breakthrough pressure depends only on the radius of the tube and the effective surface tension. In certain embodiments, when θ_{adv}+θ_{flange}≥180° for a curved or angled flange, the pressure barrier becomes

$$Δ P ( z ) = \frac{2 {γ}_{eff}}{{r}_{int} ( z )} .$$

In this embodiment, rᵢₙₜ(z) is larger than the radius of the lumen due to the angled flange, thus decreasing the pressure barrier compared to sharp flange angles. For hydrophilic conduits, certain embodiments shall consider the first breakthrough pressure and a second breakthrough pressure. The first breakthrough pressure is the pressure at which the fluid exits the conduit, and the second breakthrough pressure is the pressure at which the liquid wets the entire area of the flange, as shown in FIG. 11D.

As described herein, the dimension and shape of the tube, flange, and surface properties of the conduit material play a significant role in guiding or suppressing the flow of liquids.

In certain embodiments, membranes with pore sizes (rₚₒᵣₑ) ranging from hundreds of nanometers to tens of microns are incorporated into the tympanostomy conduits to increase the pressure barrier associated with fluid transport. The discussion above holds, with rᵢₙₜ=rₚₒᵣₑ, and only highly wetting liquids are able to permeate the ear-conduit. This effect could be beneficial for allowing, for example, silicone oil transport carrying medication while reducing and/or preventing the transport of aqueous liquids into the inner-ear cavity.

In certain embodiments, the pores can rapidly and repeatedly open and close, enabling precise, dynamic modulation of gas/liquid sorting and controllable separation of a three-phase system of air/water/oil mixture, complex solutions and suspensions such as proteins and blood. In certain embodiments, a liquid-filled pore can provide a gating strategy which offers a unique combination of dynamic and interfacial behaviors, according to US 2018/0023728 published on Jan. 25, 2018, the contents of which are incorporated herein by reference. These embodiment can be used to design gated transport systems starting from a wide variety of pore sizes, geometries, and surface chemistries as well as gating liquids, according to certain embodiments. In certain embodiments, the substrate can contain pores that are about in average 10 nm to about 3,000 microns in size or of any combination of sizes in between, such as 20 nm to 2 microns, 100 nm to 10 microns, 100 nm to 1.2 microns, 80 nm to 1 micron, 200 nm to 5 microns, 10 nm to 10 microns, and 100 nm to 50 microns.

In certain embodiments, the geometry and chemistry of the device that is built from a dynamic, environmentally responsive material can be temporarily changed by applying the external stimulus, such as light, temperature, or chemical environment, to allow for a provisional transport or delivery through the tube, according to certain embodiments, and as discussed in further detail throughout this disclosure.

2. Surface Tension of Lubricating Liquids

In certain embodiments, lubricating liquids can alter the surface tension of the surface of the conduit. In certain embodiments, conduit low surface tension lubricating liquids (˜19 mN/m) form a 0-degree advancing contact angle on tympanostomy conduits to allow for essentially barrier-less transport of oil drops through the conduit. Water droplets, depending on the presence of the lubricating liquid wrapping layer, have a much larger surface tension (60 mN/m with the wrapping layer and 72 mN/m without wrapping layer), and a high advancing angle. Thus, in certain embodiments, it can be more challenging to drive water through the conduit. Mucus, which has surface tension on the order of ˜50 mN/m, is therefore easier to transport through the conduit than water in this embodiment. The immobilized liquid interface facilitates the transport of water into the ear through the conduits with certain dimensions (<1 mm ID). The selection of lubricant can be optimized in order to reduce effective surface energy and lower the contact angle of a certain fluid in order to promote transport, or, conversely, increase the contact angle and inhibit transport, in accordance with certain embodiments. Introducing surfactants to water also alters fluid transport through the conduit, according to certain embodiments.

3. Geometry Optimization for Enhanced Preferential Flow

In certain embodiments, an optimization of conduit geometries can be performed to allow selectively preferential flow of one or more liquids. The parameters for such optimization are provided by the Young-Laplace equation governing the maximum pressure for the fluid:

$$Δ P = - \frac{2 {γ}_{eff}}{r} cos ( {θ}_{adv} + {θ}_{flange} ) ,$$

where, ΔP is the pressure difference across the meniscus of the fluid. One could modify: a) the effective surface tension of the phase in contact with air (γ_{eff}), b) the radius of the tube (r), c) the advancing angle of the three-phase front, d) lumen wall tilt angle and θ_{flange}), e) the surface properties of the tube, f) the bevel of the tube and flanges. The effective surface tension is dictated by the spreading coefficient of lubricant on the fluid: S_{LD}=γ_{DV}−γ_{DL}−γ_{LV}, where, S_{LD} is the spreading coefficient of lubricant on droplet, and γ_{DV}, γ_{DL}, and γ_{LV} are the interfacial tensions of droplet-vapor, droplet-lubricant, and lubricant-vapor, respectively. When spreading coefficient is larger than 0, it is favorable for the formation of a wrapping layer due to the minimization of energy. The effective surface tension is the lower of the values between γ_{DV}, and γ_{DL}+γ_{LV}. Such optimization can be performed for various materials, smoothened, chemically patterned, or morphologically textured of the tube in accordance with certain embodiments.

In certain embodiments, preferential flow is the preferential unidirectional flow of one material relative to another. In certain embodiments, preferential flow is the preferential unidirectional flow of therapeutic drops versus environmental water. One route for the optimization can be performed numerically by keeping the Young-Laplace pressure of an antibiotic solution constant throughout the length of the lumen. In certain embodiments, the angle of the inner surface of the conduit can be varied to maintain a constant Young-Laplace pressure. By continuously changing the flange angle (e.g., the distal angle of a distal flange or the proximal angle of a proximal flange) and radius in infinitesimal increments (dr and dθ_{flange}) one can achieve an azimuthal symmetric or axisymmetric geometry with an optimal curvature, which maintains constant Young-Laplace fluid pressure:

$$Δ P = Const = - \frac{2 {γ}_{eff}}{r} cos ( {θ}_{adv} + {θ}_{flange} ) = - \frac{2 {γ}_{eff}}{r + dr} cos ( {θ}_{adv} + {θ}_{flange} + d {θ}_{flange} ) .$$

The same pressure is realized through the conduit's lumen where θ_{adv}+θ_{flange,final}=180°. In certain embodiments, the final flange angle can be tuned by adjusting material properties. In certain embodiments, conduits with improved performance incorporating straight-angled or curved flanges can be achieved by allowing the pressure in the flange to vary by up to ±1%, 5%, 10%, 20%, 30%, 40%, 50%, 60%, 70%, 80% or any intermediate values. In certain embodiments, these shapes are determined by considering the initial pressure in the flange:

$$Δ {P}^{′} = - \frac{2 {γ}_{eff}}{{r}_{int} ( 0 )} cos ( {θ}_{adv} + {θ}_{flange} ( 0 ) ) .$$

Throughout the length of the flange z, one can impose the condition

$$Δ {P}^{′} ( 1 - x ) < \frac{- 2 {γ}_{eff}}{{r}_{int} ( z )} cos ( {θ}_{adv} + {θ}_{flange} ( z ) ) < Δ {P}^{′} ( 1 + x )$$

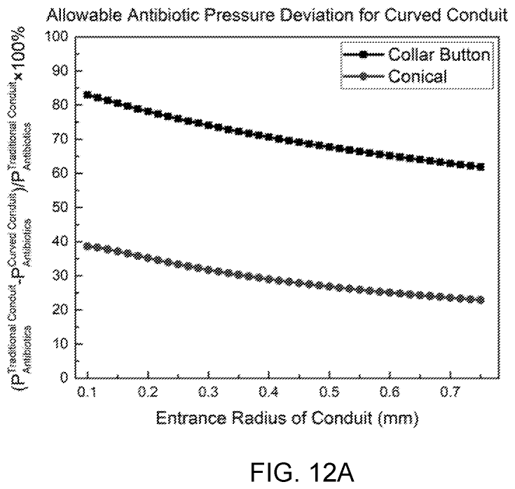

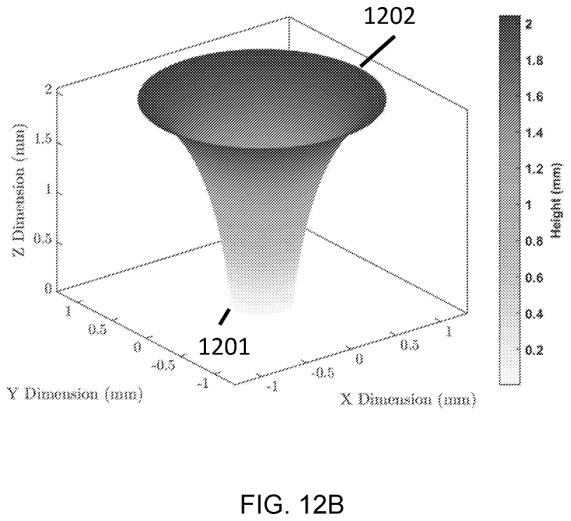

for all z until the end of the flange, where x=0.01, 0.05, 0.1, 0.2, 0.3, 0.4, 0.5, 0.6, 0.7, 0.8 and x is the allowable pressure variance in the flange. FIG. 12A shows the comparison a difference in maximum pressure of antibiotic drops flowing through an optimized conduit and a cylindrical or conical one, normalized by the maximum pressure of a cylindrical or conical conduits, respectively, to show that deviation in pressure in an optimized tube can be within ˜80% range. The flanges can be curved or straight or can made up of many straight angled sections. Pressure can also be optimized to be reduced as compared to existing analogous devices where ΔP=ƒ(z), where f(z) is a chosen function that governs the pressure difference along the tube.

In certain embodiments, the Young-Laplace pressure of a material does not vary along the tube or conduit. In certain embodiments, the Young-Laplace pressure of the material only varies by 80% or less, 70% or less, 60% or less, 50% or less, 40% or less, 30% or less, 20% or less, 10% or less, 5% or less, 2% or less, or 1% or less. In certain embodiments, the material can be the first material traveling from the distal end to the proximal end. In certain embodiments, the material can be the fourth material traveling from the proximal end to the distal end.

The flow model can be optimized through an interplay of various parameters: Young-Laplace fluid pressure (ΔP), initial radius (r), initial flange angle (θ_{flange,initial}) (e.g., the distal angle of a distal flange or the proximal angle of a proximal flange), and length of the lumen (L). These degrees of freedom can be swept and optimized to (1) maximize Young-Laplace pressure for water, (2) minimize Young-Laplace pressure for drug solution and (3) minimize deviance from the prescribed tube length. As shown in certain embodiments, in FIG. 12A, the difference between the antibiotic pressure and the water pressure was greater in curved conduits compared to collar button and conical conduits. One example of such optimized curved tube is shown in the FIG. 12B, where the tube length was constrained to 2 mm, and an exemplary radius was selected to be 0.275 mm at the proximal end 1201. This shape demonstrates a constant antibiotic pressure of 74.7 Pa throughout the tube from the proximal end to the distal end 1202.

In certain embodiments, the radii are in the range between 10 nm and 1500 μm (capillary length of water). In certain embodiments, the radii are 10 nm, 50 nm, 100 nm, 200 nm, 300 nm, 400 nm, 500 nm, 600 nm, 800 nm, 1 μm, 10 μm, 50 μm, 100 μm, 200 μm, 300 μm, 400 μm, 500 μm, 600 μm, 700 μm, 800 μm, 900 μm, 1000 μm, 1100 μm, 1200 μm, 1300 μm, 1500 μm, or any value in between. In certain embodiments, the radii are 1 mm, 2 mm, 3 mm, or any value in between

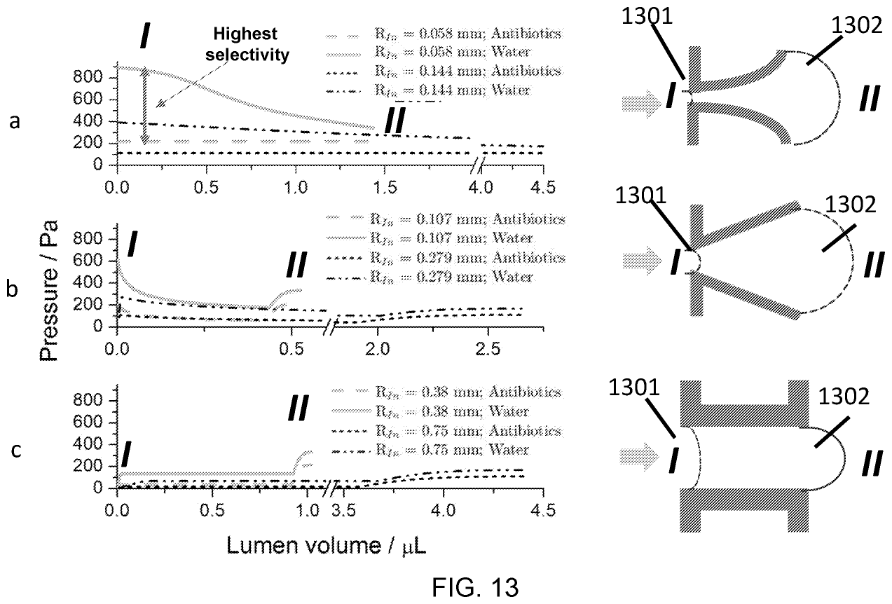

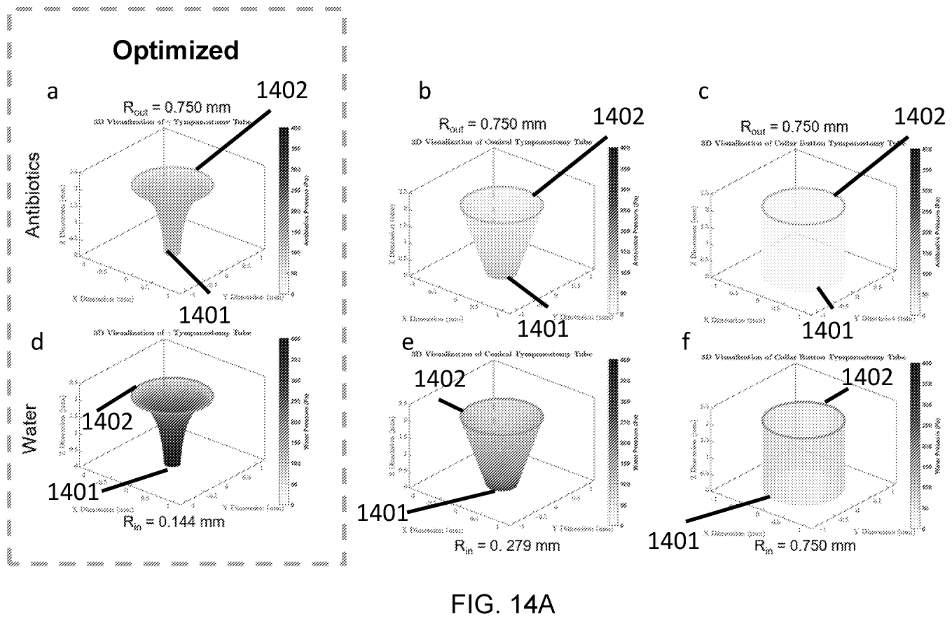

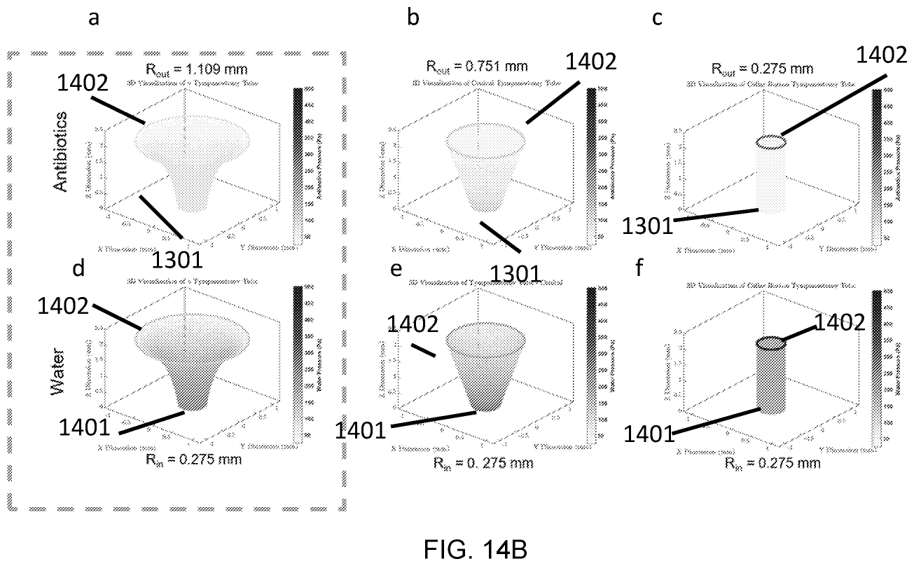

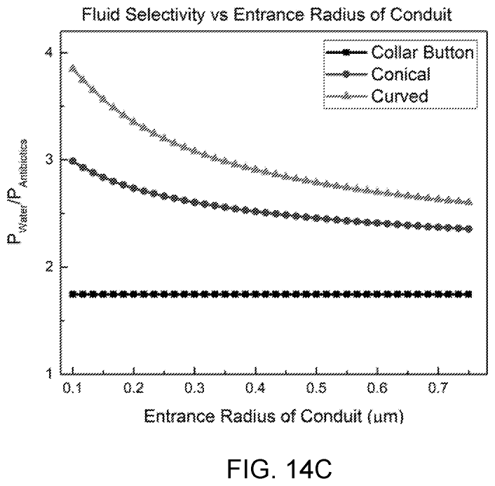

Advantageously, tubes with an optimized design have significantly smaller (by 1.5-10 times) radii but similar or much lower maximum antibiotic Young-Laplace pressures and higher water Young-Laplace pressures as compared to any of control cylindrical or conical tubes with larger radii independently of its shape, as shown in the FIG. 13. Thus, the tube can be smaller, less invasive and less damaging, but still achieve, for example, the benefit of passing antibiotics through to the inner ear and without allowing water to pass. Furthermore, tubes with a curved, optimized design based on tuning the interplay of Young-Laplace equation parameters provide an exceptional selectivity for a desired fluid. For example, in some embodiments, the tubes with an optimized curved design, for example as shown in FIG. 13 (view a) with the radii of 0.058 and 0.144 mm demonstrate the largest difference between the pressures of water and antibiotic drops. Note, that the antibiotic pressure stays constant throughout the length of the tube from the proximal end 1301 to the distal end 1302 and shows no jumps in pressure as compared to larger conical (FIG. 13 view b), (e.g. Baxter Beveled shape, r=0.107 mm and r=0.279 mm) and much larger cylindrical (FIG. 13 view c) (e.g. Collar Button shape, r=0.380 mm and r=0.750 mm). In certain embodiments, highest selectivity of the optimized curved tubes can also be seen in the FIG. 14A and FIG. 14B, in which the most intense pressure difference is seen for the curved geometry as compared to conical and cylindrical ones with same exit radii of the tube, yet the smallest entrance radius of the curved tube (again indicating that the optimized tubes can be considerably smaller in size yet preserve all the benefits of commercial tubes).

Similarly, in some embodiments the tubes can be optimized for a broad variety of liquids, for example water and antibiotics. For the calculations for FIG. 14A and FIG. 14B, the interfacial tensions (IFTs) of the fluids were measured using the goniometer using the pendent droplet method. The IFTs for water are γ_{DV}=72.3 mN m⁻¹, and γ_{DL}=44.5 mN m⁻¹; the IFTs of antibiotic drops are γ_{DV}=41.43 mN m⁻¹, and γ_{DL}=25.00 mN m⁻¹; the lubricant-vapor IFT (γ_{LV}) is 18.8 mN m⁻¹. For the simulations, the advancing angle of the three phase front was taken from Young's equation:

$${θ}_{adv} = {cos}^{- 1} ( \frac{{γ}_{LV} - {γ}_{DL}}{{γ}_{eff}} ) .$$

In certain embodiments, shown in FIG. 14A, by design the maximum Young-Laplace for antibiotic (FIG. 14A views a-c) drops can be adjusted to be same for all oil-infused tubes (75 Pa): collar button or cylindrical (FIG. 14A views c and f), conical (FIG. 14A views b and e), and curved (FIG. 0.14A views a and d). The simulations show that the corresponding pressure of water (FIG. 14A views d-f) is highest for the curved geometry. This indicates the highest pressure difference is seen for the curved geometry as compared to conical and cylindrical tubes with same exit radii 1402 of the conduit (with different entrance radii 1401), in accordance with certain embodiments. In certain embodiments, the entrance radii 1401 is at a proximal side of the conduit, and the exit radii 1402 is at a distal side of the conduit to prevent water from entering the inner ear but permit antibiotics to pass through the conduit.

In certain embodiments, shown in FIG. 14B, the entrance radii 1401 can be selected to be the same for all shapes (with thee exit radii remaining constant): curved (FIG. 14B views a and d), conical (FIG. 14B views b and e), or cylindrical or collar button (FIG. 14B views a and d). FIG. 14B maps the simulated Young-Laplace pressure of antibiotics (FIG. 14B views a-c) and water (FIG. 14B views d-f) along the length of conduit of various geometries. The simulations show that the optimized conduits that have the same conduit entrance radius as control tubes of various shapes (e.g. Baxter Bevel and Collar Button) show higher fluid selectivity at the maximum pressure point within the lumen, as seen in FIG. 14B. The fluid selectivity is determined from the normalized Young-Laplace pressure difference between the fluids of transport. For the case of unidirectional transport for the preferential flow of therapeutic drops compared to water, the optimized curved tube design has a higher fluid selectivity of 3.1 compared to conical shape of 2.5 and cylindrical shaped of 1.5. FIG. 14C. shows certain embodiments where the non-limiting range of selectivity (ratio of maximum water pressure to maximum antibiotic pressure) for the optimized curved designs is 3-4 as compared to lower selectivities of the cylindrical and conical conduits. In certain embodiments, selectivities can be further optimized for other specific fluid examples to achieve selectivity between 0.0001 and 10. In certain embodiments, the selectivity is between 5 and 6.

In certain embodiments, the selectivity between materials, such as the first and second materials, is in the range of 1 to 1.2, 1.2 to 1.5, 1.5 to 1.7, 1.7 to 2, 2 to 3, 3 to 4, 4 to 5, 5 to 6, 6 to 8, 8 to 10, 1 to 10, 1.2 to 10, 1.5 to 10, 1.7 to 10, 2 to 10, 3 to 10, 4 to 10, 5 to 10, 6 to 10, 1 to 10, 1.2 to 8, 1.5 to 6, 1.7 to 5, and 2 to 4.

In certain embodiments, the difference between the Young-Laplace pressure of two materials, (such as the difference between the first, third or fourth materials and second material), is greater than 1 Pa, greater than 5 Pa, greater than 10 Pa, greater than 25 Pa, greater than 50 Pa, greater than 100 Pa, or in the range of 1 MPa to 1000 MPa, 5 MPa to 1000 MPa, 10 MPa to 1000 MPa, 25 MPa to 1000 MPa, 50 MPa to 1000 MPa, 100 MPa to 1000 MPa, or 500 MPa to 1000 MPa.

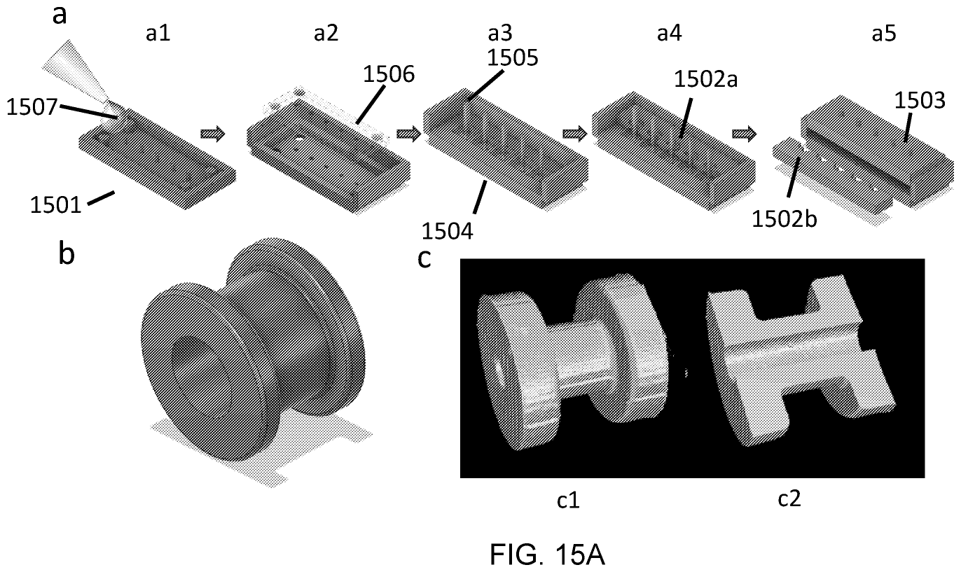

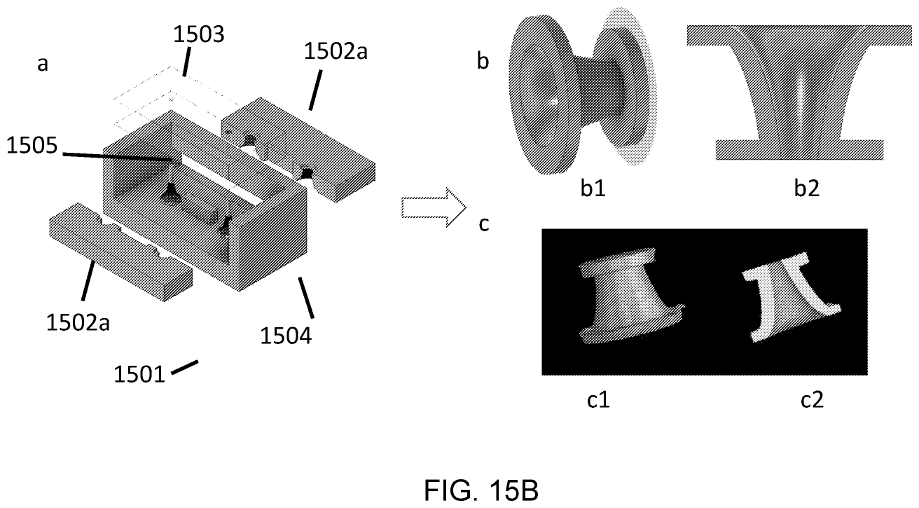

FIG. 15A shows the additive manufacturing and injection molding fabrication method used to produce the tympanostomy conduits with cylindrical “Collar Button” shape, according to certain embodiments. In certain embodiments, this tube can be re-designed after molding to have desired surface modifications, e.g. a lubricious overlayer for enhanced antifouling properties and enhancement of fluid mobility on the surface of the conduit, improvement of smoothness, and reduction of pinning, or a different mono- or multilayer functional coating, or, textural modifications, e.g. patterned morphology in accordance with certain embodiments described below. FIG. 15A (view a, including views a1-a5) is a schematic illustration of injection molding manufacturing of tubes with cylindrical shape. The mold 1501 design consists of four 3D-printed parts: two side parts 1502a, 1502b with concavities that are designed to clasp around a custom pin 1505 of a desired diameter that forms the conduit's lumen; two “top” 1503 and “bottom” 1504 parts with rectangular wells that hold flat polydimethylsiloxane blocks 1506 (PDMS, Sylgard 184, Dow Corning). The two PDMS blocks (5:1=base-to-crosslinker ratio) maintain the pin in proper position and provide a tight seal between the pin and the surrounding parts. In some embodiments, as shown in FIG. 15A (view a1), the bottom part of the mold is filled with a curable polymer 1507, for example, PDMS. The PDMS blocks can be added (FIG. 15A view a2) to hold the pins in place (FIG. 15A view a3). The first side part is added to define the outer surface of the conduits, followed by the second side part and the top part. In some embodiments, the curable polymer is cured by exposure to heat, light, or a cross-linking agent. FIG. 15A (view b) shows a three-dimensional schematic of a conduit according to certain embodiments. FIG. 15A (view c1 and c2) shows the side view of the conduit and its cross section, according to certain embodiments.

FIG. 15B shows manufacture of an exemplary embodiment of tympanostomy conduits with an anisotropic curved shape. FIG. 15B (view a) is a schematic illustration of injection molding manufacturing of tubes with a curved shape, with like reference numerals to FIG. 15A referring to like elements. The molds were fabricated by additive manufacturing. FIG. 15B (views b1 and b2) shows a three-dimensional schematic of a conduit according to certain embodiments. FIG. 15B (views c1 and c2) shows the side view of the conduit and its cross section, according to certain embodiments.

The following example further describes and demonstrates embodiments within the scope of the present invention. The examples are given solely for the purpose of illustration and are not to be construed as limitations of the present invention, as many variations thereof are possible without departing from the spirit and scope of the invention.

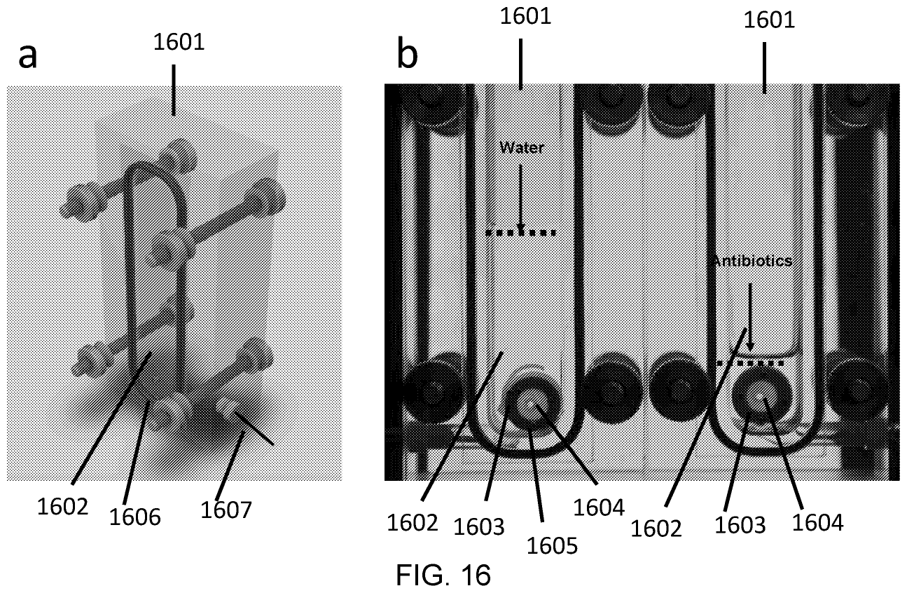

In some embodiments, experimental results of tubes with approximately the same inner radius of 0.275 mm and length of 2 mm shows that the Young-Laplace pressure for fluid transport of antibiotic drop mimicking solution is reduced significantly more than for water for curved-infused tubes compared to Collar Button non-infused tubes as measured using the apparatus as shown in FIG. 16. The apparatus, consisting of an acrylic chamber 1601, polyethylene-terephthalate-based membrane 1602 and rubber blend gasket 1603 held with two magnetic rings 1605, used for measuring the Young-Laplace pressure, as shown in the FIG. 16. The apparatus was washed with isopropyl alcohol and dried with compressed air prior to each experiment. The tube 1604 was inserted through a circular hole with diameter of 0.20-0.75 mm into the rubber gasket to prevent leakage of tested fluid; a tight seal between the gasket and the membrane was ensured through two ring-shaped magnetic rings 1605. The membrane with the gasket was then placed onto the chamber and secured with two rubber O-rings 1606 on either side of the membrane for a tight seal. A needle 1607 was placed into the rubber plug to pump the fluid into the chamber. Fluid was dispensed using a syringe pump (Harvard Apparatus PHD Ultra™), delivering an initial 3 mL of a fluid at a rate of 1000 μL min⁻¹, then an additional 3 mL at a rate of 500 μL min⁻¹, and the remaining fluid at a rate of 250 μL min⁻¹. The water level was captured using a camera. ImageJ was then used to determine the height (h) of fluid on the image when the fluid exits the tube, using h the hydrostatic pressure (P) of the fluid was calculated using P=ρgh, where ρ is the density of the fluid and g is acceleration due to gravity.

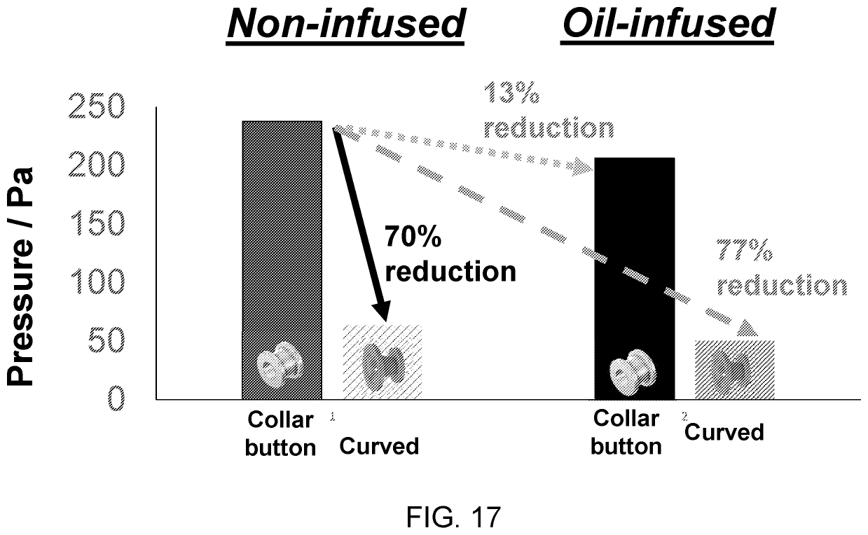

The pressure reduction from a non-infused Collar Button tube to an optimally curved tube according to certain embodiments with infusion for antibiotic drop-mimicking solution is 77%, as seen from the FIG. 17. Hence even at radii scales of the same value, the fluid selective transport property of the curved design according to certain embodiments will be preserved. FIG. 17 shows a drastic reduction of pressure of 70% and 77%, respectively for non-infused and infused curved samples as compared to non-infused Collar Button Cylindrical tube, as indicated with the arrows. Oil-infused Collar Button tubes show a 13% reduction in pressure as compared to non-infused Collar Button tube, indicating that, in certain embodiments, adding a lubricious layer also reduces the pressure due to modification of the effective surface tension of the liquid, and preventing pinning. In certain embodiments, conduits can have reduced pressure and guided fluid flow based on geometry, without a lubricating liquid.

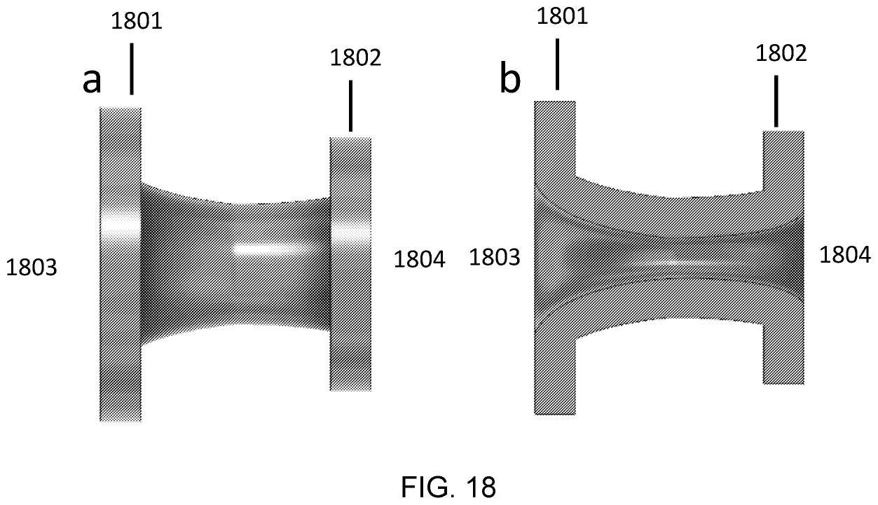

In certain embodiments, shown in FIG. 18, the bidirectional design is desired, and the conduit can, for example, be constructed by the junction of a proximal curved segment 1801 and a distal curved segment 1802. Each segment is optimized for the fluid transport of a particular set of fluids. FIG. 18 demonstrates a non-limiting example of such an “hourglass” geometry and its side (FIG. 18 view a) and a cross-section (FIG. 18 view b). The length of the entrance segment is optimized for administering antibiotic ear drops and minimizing the entrance of water from the middle ear 1803, whereas the length of exit segment is optimized for excreting middle ear 1804 effusion.

Such properties can enable the topical administration of drugs, such as antibiotics, which currently could not be delivered effectively through existing devices as they cannot pass from a proximal end of the tubes through the distal end to the inner ear.

In certain embodiments, the conduit provides for controllable flow of aqueous humor of the eye from the anterior chamber into subconjunctival spaces to reduce intraocular pressure (IOP) in controllable fashion and reduce the need for further treatments in glaucoma patients.

In certain embodiments, the use of a curved geometry for a conduit transporting aqueous humor of the eye will reduce the minimum gradient of pressure across the anterior chamber and subconjunctival spaces required for flow. In certain embodiments, this reduction in pressure gradient allows for lower difference in opening and closing pressures of the AGV shunt and reduced opening pressures. Higher opening pressures leads to inadequate IOP control in long term placements and can be worsen from increased flow resistance from tissue around the glaucoma drainage device.

In certain embodiments, conduits have of switchable slippery surfaces. In these embodiments, the conduit can switch between slippery and non-slippery states to restrict surplus flow of aqueous humor preventing postoperative hypotony.

In certain embodiments, the conduit has switchable pinning sights for controllable flow of fluid. In certain embodiments, when eye pressures are in normal ranges of 12-20 mmHg, the pinning sights are inactivated. In certain embodiments, when the eye pressure drops below 12 mmHg, the flow of aqueous humor is inhibited by a stimuli which increases the effect of pinning sights and naturally allows the eye pressure to equalize into normal ranges.

In certain embodiments, the conduit has surface modified lumen in which the flow is reduced or completed restricted via stimuli to prevent postoperative hypotony.

4. Chemical and Geometric Patterns for Enhanced Preferential Flow

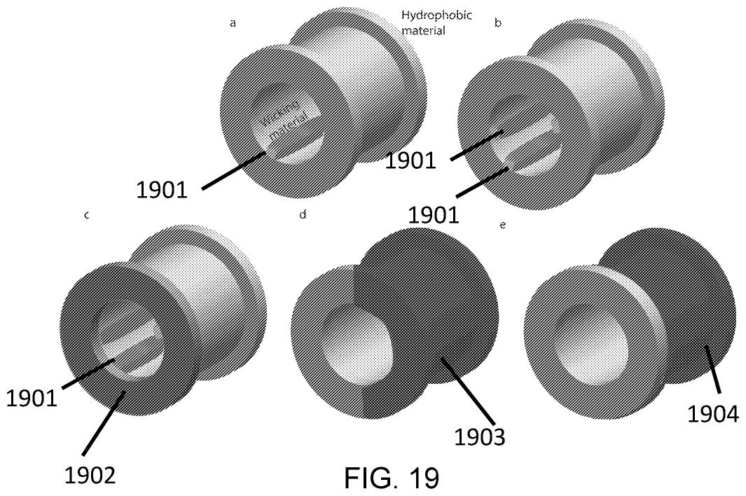

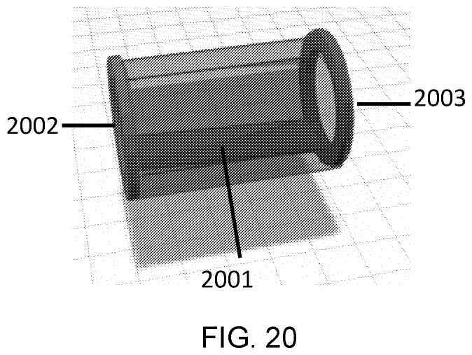

In certain embodiments, tympanostomy conduits or subannular ventilation conduits contain wicking materials or chemical gradients on the flanges of the conduit to guide or enhance the flow of fluids. In certain embodiments, the chemical gradient increases the effective surface tension of the conduit, and in other embodiments, the chemical gradient decreases the effective surface tension of the conduit. In certain embodiments, multimaterial printing or other manufacturing methods can allow two or more materials (shown in different shades of gray) to be patterned into the same device for gathering or wicking the liquid as shown in the designs in FIG. 19 (views a-e). In some embodiments, the conduits include non-swellable and swellable hydrophilic materials as well as non-swellable and swellable hydrophobic materials. Normally, the flanges on the ends of a tympanostomy conduit serve primarily to hold the conduit in place in the hole created in the tympanic membrane. Along with surface chemistry, the physical structure of the tympanostomy conduits can be varied to incorporate flanges with these specific chemistries to guide the fluid flow toward the other side of the conduit. In certain embodiments, guided flow utilizes a funnel, flange, a flange with differing chemistry, or a flange with a gradient in surface chemistries moving toward the hole in the conduit. In certain embodiments, such gradients can also be included on the inner surface of the conduit, as shown in FIG. 20. In some embodiments, the chemical pattern 2001 can include a chemical gradient, with a first density of the chemical at the distal end 2002 of the conduit and a second density of the chemical at the proximal end 2003 of the conduit to enhance flow along the length of the conduit. In other embodiments, the chemical pattern can have a shape that enhances flow through the conduit. For example, the width of the chemical pattern can increase from the proximal end to the distal end. A second chemical pattern can also be included, with changes in shape, density, or other parameters from the distal end 2002 to the proximal end 2003. In certain embodiments, the first chemical pattern can be hydrophobic, and the second chemical pattern can be hydrophilic. In certain embodiments, the distal end 2002 is hydrophobic and the proximal end 2003 is hydrophilic, and transport of water from the proximal to distal end is enhanced. In certain embodiments, depending of the lipophilicity or surface tension of the hydrophobic proximal end, it can promote antibiotic transport from distal end to proximal end. Depending on the composition of the effusion, the flow will be preferential from distal end to proximal end. In other embodiments, the distal end is hydrophilic and the proximal end is hydrophobic.

FIG. 19 is a schematic illustration of chemically patterned tympanostomy conduits, according to certain embodiments. In some embodiments, chemical patterns can include wicking layers 1901 on the inner surface of the conduit optimized for transport of fluid. In some embodiments, the wicking material is porous, and the fluid moves through the wicking material by capillary action. FIG. 19 (view a) depicts a single wicking layer, FIG. 19 (view b) depicts multiple wicking layers, FIG. 19 (view c) depicts a wicking flange material 1902 connected to multiple wicking layers inside the conduit, FIG. 19 (view d) depicts a wicking layer comprising a portion 1903 of the conduit, and view FIG. 19 (view e) depicts wicking layer placed selectively on the inner surface or outer surface 1904 of the conduit. In certain embodiments, the chemical gradient can be placed on a center portion of the conduit.

Non-limiting examples of materials for a wicking layer include hydrophilic polymers or hydrogels, such as poly(ethylene glycol), poly(acrylic acid), poly(N-isopropoylacrylamide) (PNIPAM), poly(vinylpyrrolidone), poly(2-oxazoline), cellulose, or alginate. Materials could also include hydrophobic polymers, such as poly(dimethyl siloxane), polyurethanes, acrylics, carbonates, polyesters, polyethers, or fluorocarbons that can have surface modifications. The material could also be proteins, including collagen, gelatin, fibronectin, laminin, or any RGD-conjugated natural or synthetic material.

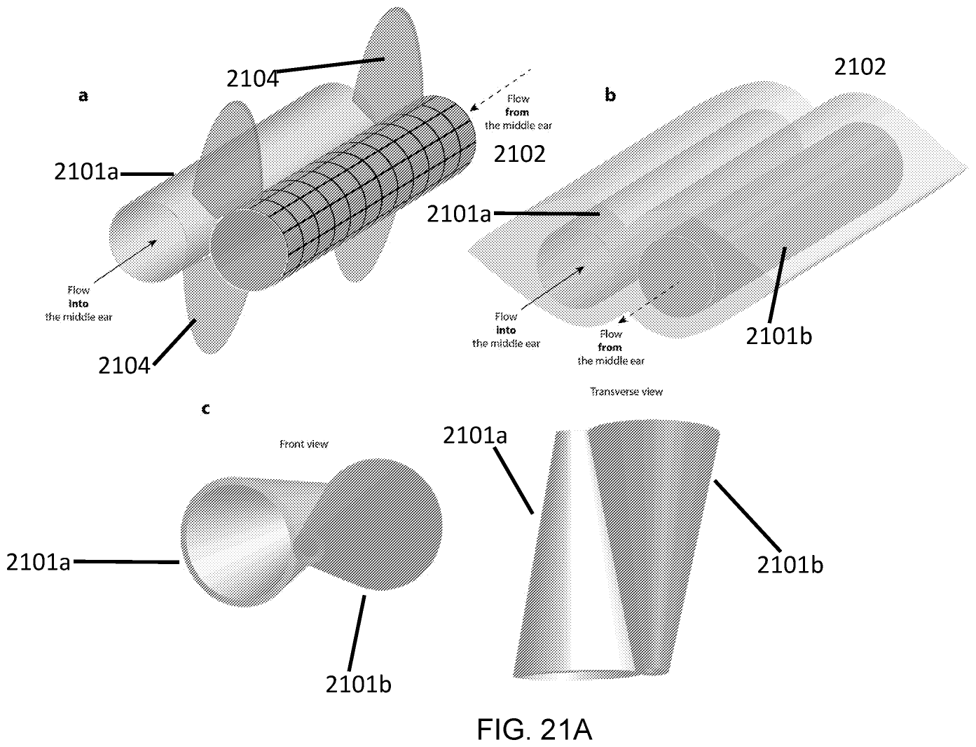

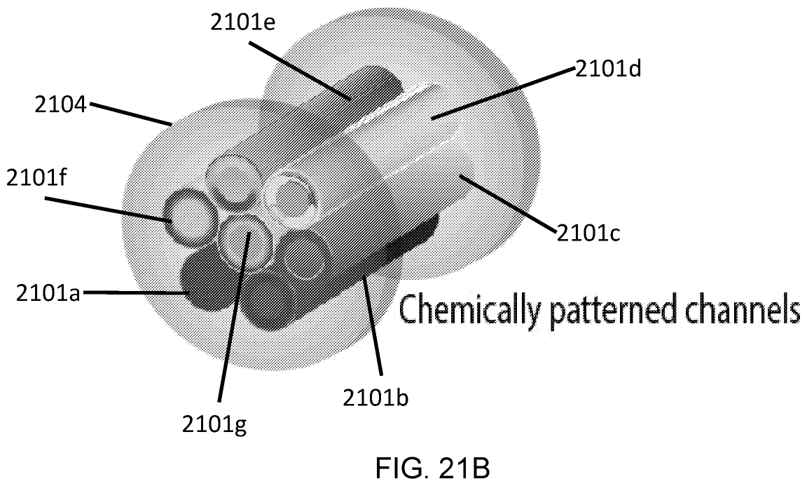

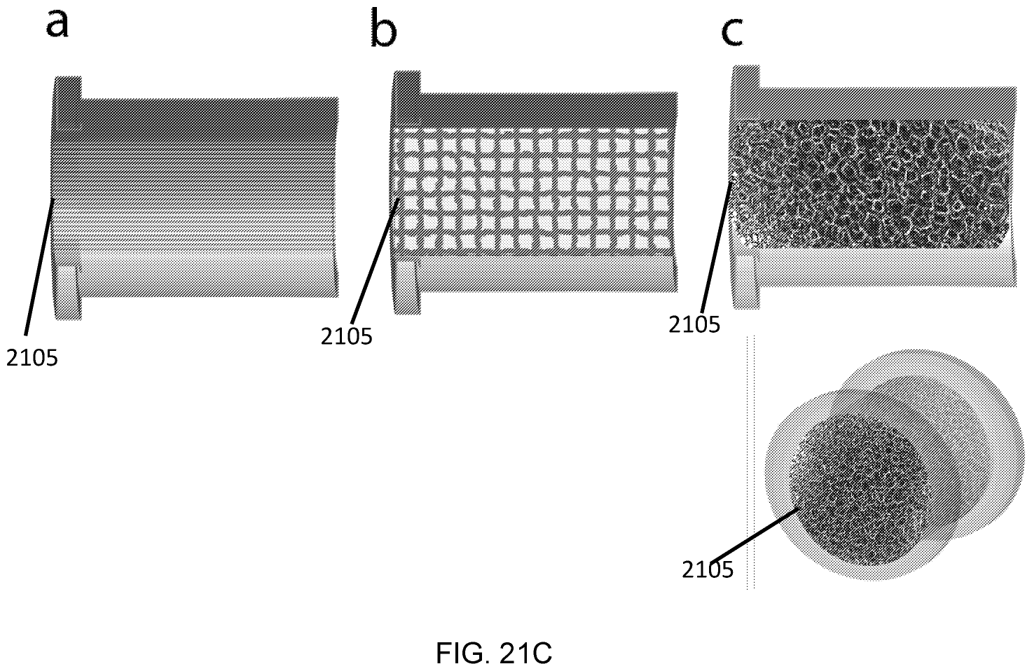

In certain embodiments, a solution includes a dual-channel conduit with patterned chemical properties, for example as shown in FIG. 21A, or multi-channel with patterned chemical properties as shown in FIG. 21B. In certain embodiments, each different channel is optimized with different patterned chemical properties for the transport of different liquids, either into or out of the ear. For example, as seen in FIG. 21A (view a) a first channel 2101a has surface chemistry and architecture optimized to transport mucus out of the middle ear space 2102, while a second channel 2101b has surface chemistry and architecture optimized to transport certain antibiotic droplets into of the middle ear space. In certain embodiments, these channels can be combined with or without flanges 2104 that keep the conduits in place. In some embodiments, each conduit has its own flange, and in other embodiments, shown in FIG. 21A (view a) a dual-channel conduit has one flange on each end. In certain embodiments, can or cannot have a conical geometry to specify the flow in certain directions, as seen in FIG. 21A (view c). Flanges can also be designed specifically to wick in or out the fluid of interest at the site of entrance or exit. In addition, as shown for example FIG. 21B, a conduit can include tubes 2101a-g hat can have a variety of different chemical properties to facilitate selective guided transport of a variety of different fluids in or out of the ear.

In certain embodiments, geometric patterns can be used for preferential flow. In certain embodiments, the geometric pattern increases the advancing angle and contact angle hysteresis of a liquid entering the conduit, and in other embodiments, the pattern decreases the advancing angle and contact angle hysteresis of a liquid entering the conduit. In certain embodiments, the geometric pattern increases the advancing angle and contact angle hysteresis of a liquid entering the conduit. In other embodiments, the pattern decreases the advancing angle and contact angle hysteresis of a liquid entering the conduit. In certain embodiments, the geometric pattern can induce the Cassie-Baxter, Young-Laplace or Wenzel states, or other intermediate states. In certain embodiments, the geometric pattern is disposed on the outer or inner surface of the conduit. In certain embodiments, the geometric pattern created by surface topography, for example surface roughness, grooves, ridges, indentations, micropillars, microridges, pores, or other 3D tessellations.

In certain embodiments, as can be seen in, for example, FIG. 21C, a conduit includes a porous material within the lumen 2105. In certain embodiments, the porous material can be an array of channels 2101a-g (FIG. 21C view a), or three-dimensional periodic (FIG. 21C view b) or three-dimensional aperiodic (sponge-like) interconnected network of pores of sizes ranging from 0.01 to 1000 μm, allowing for propagation of air and fluids (FIG. 21C view c). In some embodiments, the channels are oriented parallel to the length of the channel as shown for example in FIG. 21C view a. In some embodiments, the three-dimensional network of interconnected pores is isotropic. In other embodiments, the three-dimensional network of interconnected pores is anisotropic such that the anisotropy allows a fluid to travel along the length of the lumen. For, example the pores can have higher connectivity in the axis along the length of the lumen. In other embodiments, the conduits can be composed of geometrically-patterned channels, macro-porous channels, and micro-porous channels.

5. Use of Gravity for Preferential Flow

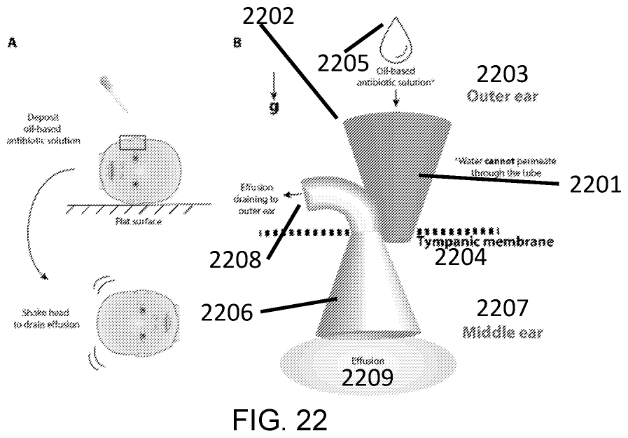

In certain embodiments, gravity plays a role in trying to transport the antibiotic droplets into the middle ear and the mucus out of the ear, for example as shown in FIG. 22 (and as discussed with respet to FIG. 18). In certain embodiments, a first conduit 2201 is provided with a conical flange 2202 on the exterior side (outer ear) 2203 of the tympanic membranes 2204 such that antibiotic droplets 2205 or other oil-based solutions or other therapeutics can only enter when the patient is held with their head horizontally. In certain embodiments, this design allows droplets to enter while reducing and/or preventing environmental water entering in most bathing and swimming situations. On the other end, a second conduit 2206 connecting the middle ear space 2207 with a hose-like structure 2208 leading out of the tympanic membrane to drain effusion 2209 into the external auditory canal. To better remove this fluid, in certain embodiments the patient can be placed laying down horizontally on their other side to encourage the fluid to flow out into the middle ear space. In certain embodiments, this opening or hose-like structure 2208 is curved to the side to reduce and/or prevent reentrance through the other conduit or water getting into that end. In certain embodiments, application of positive or negative pressure through the introduction of air, gasses, or liquids, could be used to aid in transport. In certain embodiments, these designs can also have additional gating mechanisms, as described below.

In certain embodiments, the fluidic properties can be achieved or enhanced by synergistic utilization of shape/size change benefit from FIG. 1B, for example via muscle-like contraction/expansion of the lumen, or bioinspired approach mimicking the mechanism of shape change of the proboscis of butterflies, ovipositor of insects, or beak of shorebirds.

E. Tympanostomy Conduits with Pinning to Reduce and/or Prevent Environmental Water Entrance

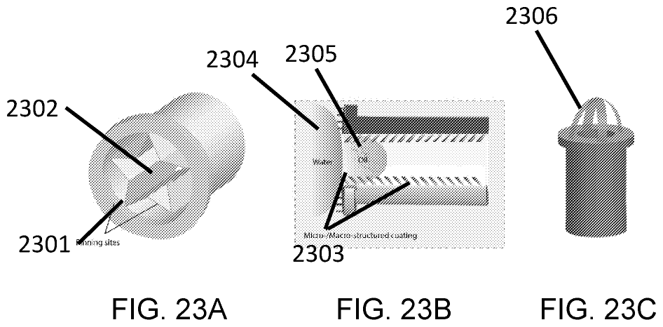

In certain embodiments, environmental water can be reduced and/or prevented from entering by increasing the pinning area for the water droplet, for example as shown in FIGS. 23A-D). In some embodiments, pinning sites increase surface tension at the entrance or the conduit. In certain embodiments, this can be accomplished by creating an opening with many angles and different corners 2301, such as a star-shaped lumen 2302 (FIG. 23A) and/or a cage-like design 2306 around the lumen (FIG. 23C). An example of a conduit with pinning sites created by lumen shape and located at the cusps within this non-limiting segmented geometry, is depicted in FIG. 23A. In certain embodiments, this pinning involves having small hair-like features 2303 coating the flanges and/or interior of the conduit. Environmental water droplets 2304 or oil droplets 2305 are pinned on these corners instead of traveling through the lumen into the middle ear space. An example of a conduit with pinning cites created by modification of lumen surface is depicted in FIG. 23B. In other embodiments, addition of a cage-shaped handle on top of the conduit or inside the lumen reduces and/or prevents environmental fluids from entering the conduit. An example of a conduit with pinning sites created by cage-shaped handle on top of the conduit or inside the lumen is depicted in FIG. 23C. In certain embodiments, eardrops can be designed to overcome these pinning effects, either through using surfactants, organic solvents, or oil-based droplets. In some embodiments, the surfactants, organic solvents, or oil-based droplets reduce the surface tension at the pinning sites.

F. Replenishment and Administration of the Lubricating Liquid to the Conduits

In certain embodiments, the lubricant drops can be administered to replenish the reservoir and re-create the anti-bacterial and improved transport properties of the conduit when the lubricating oil on the surface of the device is exhausted. For the tympanostomy conduits, replenishment can be done, for example, by applying an otic oil-based formulation which has high or low chemical affinity to the material of the conduit to induce long- or short-term longevity of the lubricating liquid on the conduit. The administration of the lubricating liquid can be targeted towards replenishment of a) only outer or b) only inner surfaces, c) only proximal or d) only distal ends of the conduit, or e) only the flange(s), or any combination thereof. The tube material can contain pores and channels that serve as lubricating liquid replenishment reservoirs.

In other embodiments, excessive lubricating liquid can be applied that either makes the flanges slippery or makes the flanges expand, swell, twist, roll, collapse, or induces appearance of periodic or aperiodic arrays of features (wells, bumps, holes, etc) or is used to enable controlled extrusion of the tubes at a desired timepoint. In certain embodiments, controlled extrusion can be done by inducing a size or shape transformation of the outer surface of the conduit, or peeling off an external thin layer around the conduit. More details are in additional sections of this disclosure.

### V. Shape Change for Minimal Invasiveness and Tissue Damage

In certain embodiments, tympanostomy conduits and/or subannular conduits can be designed to be minimally invasive and avoid tissue damage. While the following description includes certain embodiments relating to tympanostomy conduits and/or subannular ventilation conduits, the designs can be used in other medical or non-medical applications, such as such as microfluidic, membrane, bioreactors, transport of coolant and other chemicals through machinery, drainage of waste products from reactions, sensors, printing nozzles, food and beverage industry, cosmetics and perfumes, and other applications. Non-invasive designs can also be combined, for example, with antifouling, guided fluid transport, therapeutic delivery, and other aspects described throughout the disclosure. In certain embodiments, the conduit includes a shape changing or stimuli-responsive portion that facilitates insertion, extrusion, guided transport, or therapeutic delivery.

In certain embodiments, shape-changing materials change their shape and/or dimensions in response to one or more stimuli through external influences: the effect of light, temperature, pressure, an electric or magnetic field, or a chemical stimulus. In certain embodiments, the chemical stimulus is a cross-linking agent or a swelling agent. In certain embodiments, the swelling agent is the lubricating liquid. In certain embodiments, the conduit has a first configuration before exposure to the stimulus and second configuration after exposure to the stimulus.

A. Tympanostomy Conduits with Shape-Changing Features

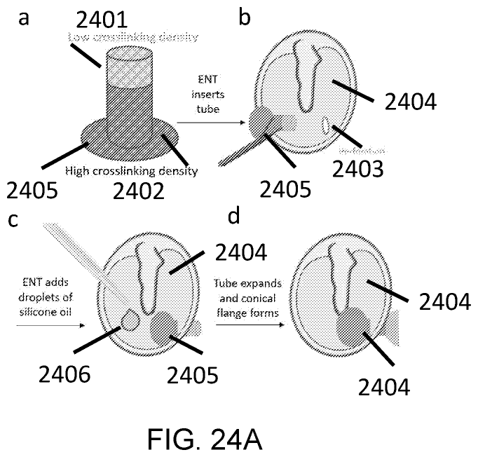

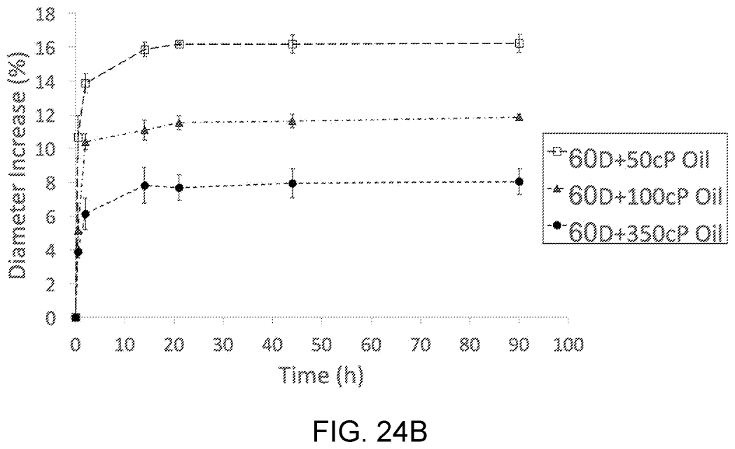

In certain embodiments, a shape/size-changing feature can facilitate the ease of insertion of the tube into the eardrum and show better post-insertion performance. In certain embodiments, this shape and dimensional change during shape change can be utilized to fabricate conduits in smaller or otherwise different dimensions that reach their desired dimension after soaking, as shown in for example in FIG. 24A. This approach can be used to achieve shapes and sizes that are otherwise difficult or more expensive to manufacture. In certain embodiments, shown in FIG. 24A (view a) the conduit has a first region with low crosslinking density 2401 and a second region with high crosslinking density 2402. This embodiment can also be beneficial for designing a shape that is small enough in a first configuration that it is easy to insert into a small perforation 2403 in the tympanic membrane 2404, shown in FIG. 24A (view b) but that can expand or have flanges 2405 in a second configuration to hold it into place (FIG. 24A, view d) better once droplets of the liquid 2406 of interest (ex: oil) are placed onto the inserted conduits as shown in FIG. 24A (view c). In certain embodiments, the shape/size-changing feature can be achieved through tube inflation and vibration or other stimuli.

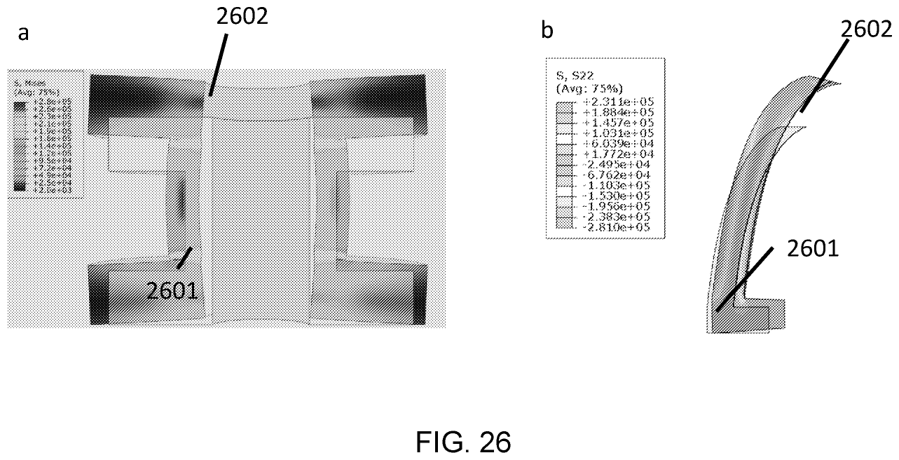

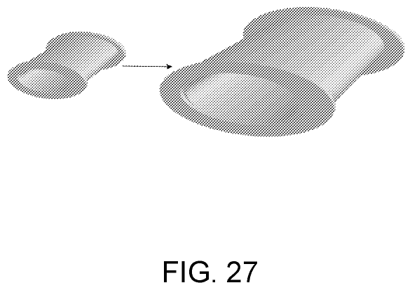

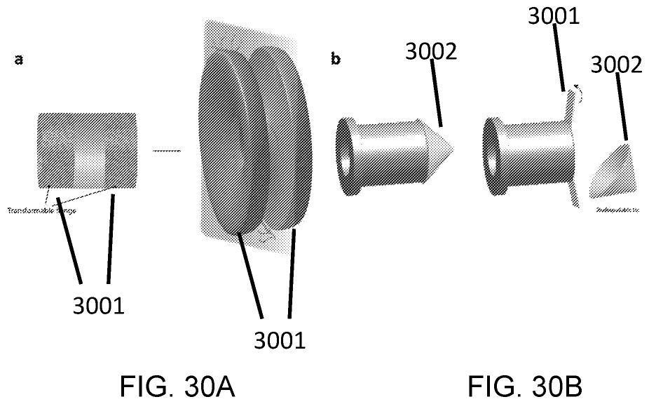

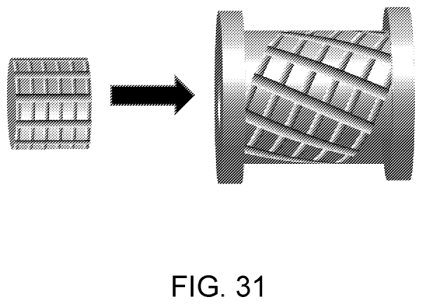

FIG. 24A illustrates how the size of the conduits is reduced prior to insertion to minimize invasiveness during the myringotomy, according to certain embodiments. In certain embodiments, the shape of the presented conduit is oval to match the elongated shape of the incision. Additional embodiments of a size-increasing shape-changing conduit is shown, for example, in FIGS. 27 and 31.

FIG. 24B. illustrates how, in certain embodiments medical grade silicone MED 4960D undergoes increase in radial dimension upon swelling at 85° C. in medical grade silicone oil with various viscosities: 50, 100 and 350 cP, as an example. The degree of swelling can be tuned by changing the silicone oil properties to reach dimension change from 0% to 20%, and generally within a range of 0% to 500%. Swelling ratio can be further tuned through modification of a combination of the tube matrix materials, cross-linking density, porosity, layer architecture, and swelling agent.

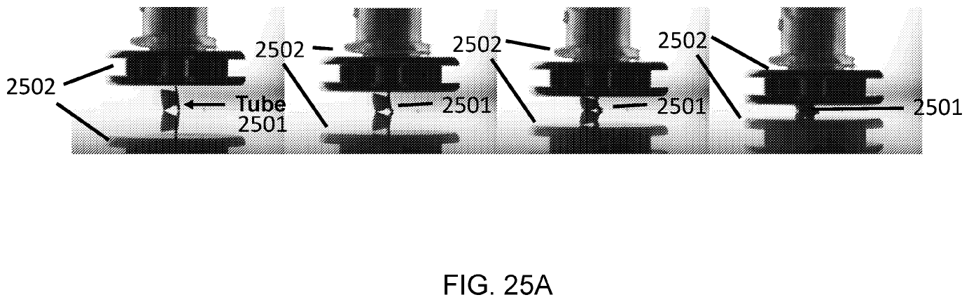

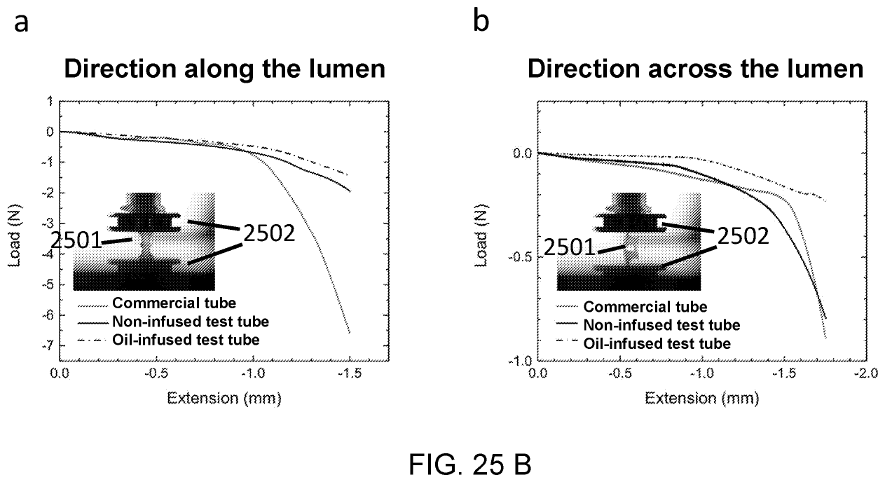

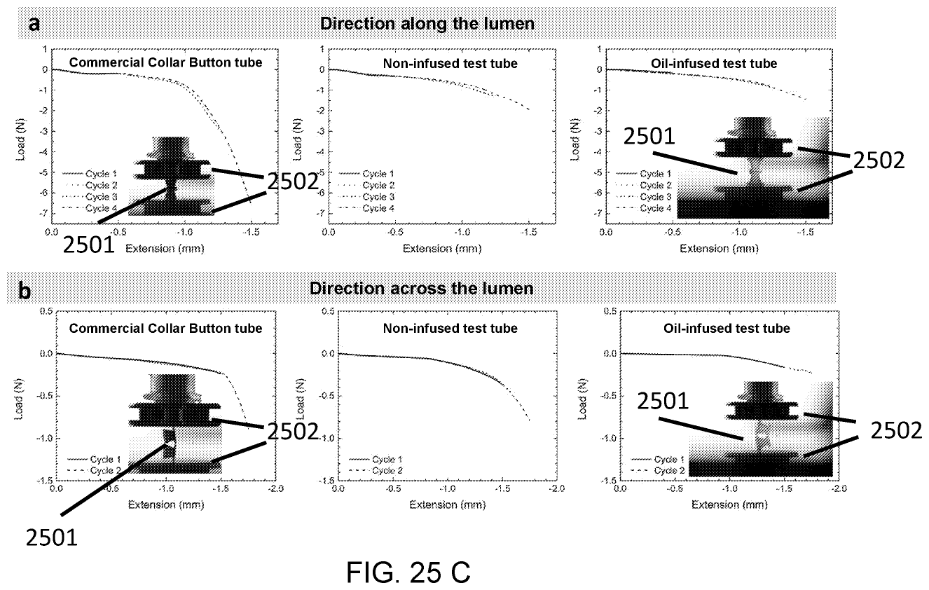

The mechanical integrity is analyzed in the FIG. 25A-25C. Compression tests were performed on an electromechanical universal testing system with a 10 N load cell. Applied load was measured during compressive extension at a rate of 0.5 mm/min. A compressive load is measured by a uniaxial compression-testing apparatus whereby the sample is placed between two flat plates, with the upper point moving toward the lower plate at a fixed rate. FIG. 25A shows compression of a silicone conduit 2501 under applied load. A compressive load is applied by a uniaxial testing apparatus 2502. FIG. 25B shows the integrity of the “test” tube along two axes, compression along the lumen (FIG. 25B a) and across the lumen (FIG. 25B b). Along the lumen, both non-infused and infused “test” tubes deform similarly, requiring significantly less force to achieve the same amount of compression. Across the lumen direction, the infused “test” tube exhibits a measurable reduction in stiffness, showing higher compressibility that can facilitate implantation and handling by surgeons. FIG. 25C shows the elasticity and fatigue resistance of the silicone tympanostomy tubes is demonstrated along two axes. Oil-infused “test” tubes maintain enhanced flexibility and compressibility over multiple loading cycles. In embodiments described elsewhere in the disclosure, in certain embodiments it is additionally desirable to increase the compressibility of the conduits to facilitate better handling by medical professionals during implant and removal. This can be achieved by design of the shape or thickness of the conduit, selection of material, addition of porosity or changes in crosslinking density, incorporation of multimaterial designs, tessellations of the overall geometry, or hardening treatments or coatings.

According to certain embodiments, the material's shape-changing behavior described can be implemented into the commercial software ABAQUS/Standard through user defined material subroutines and to solve the inverse problem, namely, to investigate the full deformation response of the final 3D tube, in order to back-calculate the original shape/size of the manufactured tube that will undergo shape transformation. This will enable a customized approach to developing customized manufacturing of the tube, according to certain embodiments. In some embodiments, the tube can be exposed to a tailored shape-changing agent for a host of desired medical indications. In order to simulate the mechanical deformation of the tube during the swelling process, a Finite Element Analysis (FEA) model of the swelling geometry was created using the commercial ABAQUS/Standard software. The FEA model was created by taking the final desired geometry as an input and solving the inverse swelling problem to obtain the fabrication geometry necessary to achieve the final geometry. The FEA model accounts for the anisotropic swelling varying linearly along the radial direction. A linear elastic material model is used for the simulations, while the strain is imposed via a uniform swelling coefficient. The model is radially subdivided into various concentric cuts with varying expansion coefficients that are fit to data that was empirically measured from the experimental procedure. Given the axisymmetric nature of the problem, the final output of the numerical model provides a cross sectional description of the geometry that can then be used for fabrication prior to the swelling operation. FIG. 26 shows the swelling stress of conduits before swelling 2601 and after swelling 2602. FIG. 26 (view a), shows cylindrical conduit geometry and FIG. 26 (view b) for curved conduit geometries, respectively, in accordance with certain embodiments.

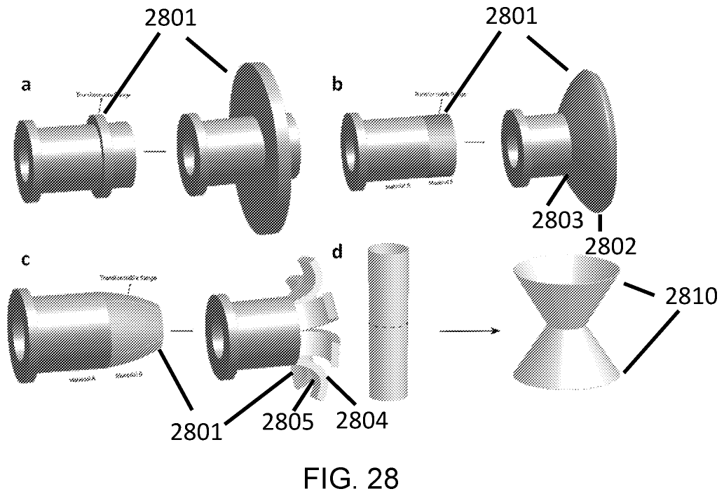

In certain embodiments, tympanostomy conduits are made of programmable materials that change shape and size on demand. The shape-changing properties are particularly beneficial for an intelligent design of flanges to minimize the invasiveness of the conduits pre- and post-myringotomy. Shape-changing materials change their shape and/or dimensions in response to one or more stimuli through external influences: the effect of light, temperature, pressure, an electric or magnetic field, or a chemical stimulus. Among these, certain materials change their shape without changing their dimensions, and other materials retain their shape but change their dimensions. Some also change both parameters at the same time. Shape changes can take place in all dimensions to equal or unequal extents. In certain embodiments, the shape-changing materials can be of thermostrictive, piezoelectric, electroactive, chemostrictive, magnetostrictive, photostrictive, or pH-sensitive nature. An embodiment of shape-changing ear conduit is demonstrated in FIG. 24, which depicts a conduit consisting of regions with materials with high and low cross-linking density changes shape when being introduced into the incision in the tympanic membrane. In this example the shape change is induced by the absorption of liquid (for example, oil) by the low-cross-linking density material. In this example, the radius of the conduit increases when exposed to a stimulus. In alternative embodiments, shown in FIG. 27, the conduit can have a uniform cross linking density and the conduit can expand uniformly. In some embodiments, shown in FIG. 28 (view d), a conduit transforms from cylindrical shape to conical (or other shape, in other embodiments), forming the flange. In certain embodiments, the amount of swelling along the length of the conduit can be controlled by controlling the density of cross-linking along the length of the conduit. Further examples of shape-changing flanges and conduit architectures are shown in FIGS. 28, 29 and 30A-B.

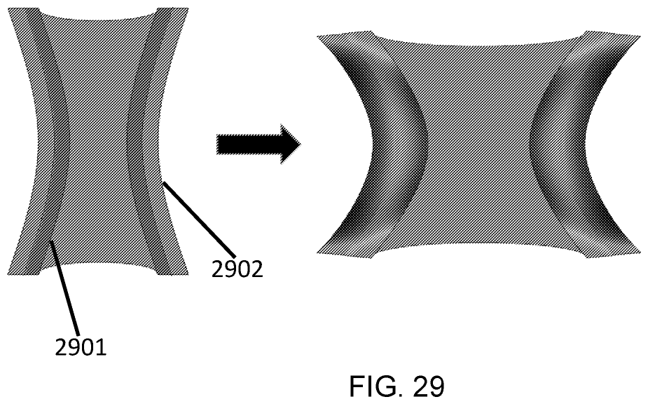

FIG. 28 is a schematic illustration of several exemplary shape-changing tympanostomy conduits with flanges 2801 that can change shape or size when exposed to a stimulus. In certain embodiments, the flanges either expand in size (FIG. 28 view a), expand in size and change shape (FIG. 29 view b), spread apart (FIG. 28 view c), or change shape into an architecture that allows for fluid transport through a funneling architecture or other guided flow design (FIG. 28 view d). In the embodiment shown in FIG. 28 (view a), the flange 2801 can expand radially into a disc shape. In the embodiment shown in FIG. 28 (view b), the flange 2801 can expand radially into a conical shape, or a curved shape. In certain embodiments, conical shape can be formed upon exposure to a stimulus if the density of cross-links varies along the length of the flange. For example, if the density is less at the proximal end of the flange 2802 compared to the density at the distal end of the flange 2803, then the proximal end can have a larger diameter. In the embodiments shown in FIG. 28A (view c), the flange 2801 can spread apart. In certain embodiments, the flanges can spread apart if the cross-linking density varies across the thickness of wall of the conduit. For example, if the cross-linking density of the inner surface of the flange 2804 is less than the cross-linking density of the outer surface of the flange 2805, the inner surface will expand more upon exposure to a stimulus, resulting in curling or spreading out of the flange. In addition, in certain embodiments, as shown in FIG. 28 (view d), a cylindrical tube can have two conical flanges 2810 that form at opposite ends upon exposure to one or more stimuli.

In certain embodiments, shown in FIG. 29, a tympanostomy conduit includes a bilayer architecture that induces a shape change. In this embodiment, the conduit is formed of two materials that have different swelling properties, for example different cross-linking densities. The two layers of the conduit can expand at different rates, resulting in a shape change. In certain embodiments, as shown in FIG. 29, if the material of the inner surface 2901 has a lower crosslinking density than the material of the outer surface 2902, the walls of the conduit will curve inward. In other embodiments, if the material of the inner surface 2901 has a higher crosslinking density than the material of the outer surface 2902, the walls of the conduit will curve outward.

In certain embodiments, shown in FIGS. 30A-B, transformable flanges 3001 expand to sandwich both sides of the tympanic membrane upon expansion (FIG. 30A) or lock onto the middle ear cavity upon expansion (FIG. 30B). In certain embodiments, shown in FIG. 30A, the conduit can have a stimuli-responsive material at the proximal and distal ends of the conduit. In this embodiment, the stimuli-responsive material expands radially upon exposure to a stimulus, forming distal and proximal flanges that sandwich the tympanic membrane. In certain embodiments, shown in FIG. 30B, transformable flange holders 3002 can be used to retain pivoting flanges 3001 that lock the tube in place. In this embodiment, the flange holder 3002 is like a cap for the flanges 3001. In certain embodiments, holders can be biodegradable, or can be actuated with external stimuli to separate from the flanges 3001. In certain embodiments, holders contain therapeutics to deliver into the middle ear. In certain embodiments, the holder 3002 can be shaped to ease insertion.

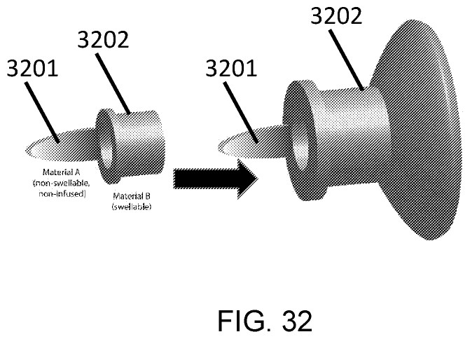

In certain embodiments, the conduit design mimics expandable stent architecture with or without the delivery balloon. For example, FIG. 31 depicts a stent-like design of a conduit that expands to form a larger architecture upon shape change. In certain embodiments, as depicted in FIG. 31 and as discussed above, the shape change can include local changes such as to create flanges at one more ends of the tube that did not exist before the shape change. In certain embodiments, the stent-like design includes a shape memory material that expands upon insertion into the tympanic membrane. In certain embodiments, an additional shape-constant material 3201 is incorporated into the conduit to facilitate the insertion of the conduit into the tympanic membrane (see an example of a magnetic handle in FIG. 32). In certain embodiments, the shape constant material forms a protrusion on the distal end of the conduit, as shown, for example, in FIG. 32. In certain embodiments, the protrusion allows the conduit to attach to a surgical tool. Upon insertion, the swellable material 3202 can expand, while the shape-constant material 3201 maintains its shape and size.

In certain embodiments, the changes of the geometry occur in the conduit or flange to induce temporal reconfigurations that improve or reduce or redirect or block liquid transport as described in the previous section. In other embodiments, dynamic structural or chemical changes can be used for the extrusion, targeted delivery, or other guided fluid transport purposes.

B. Insertion Mechanisms for Inducing Shape Change

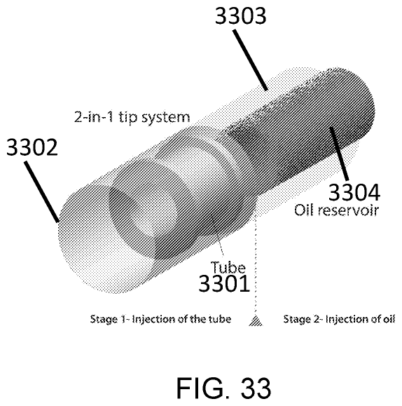

In certain embodiments, the insertion mechanism for the conduit includes two stages. An example of the two-stage insertion mechanism is shown in FIG. 33. For example, the two steps include (1) insertion of initially small conduit or tube 3301 for minimal invasiveness through the first compartment 3302 of a two-in-one tip system attached to a conduit inserter, and (2) addition of lubricant onto/into the conduit to induce antifouling, guided transport and shape change of the conduit, through the second compartment 3303 of a two-in-one tip system attached to a conduit inserter. In some embodiments, the second compartment can include a reservoir 3304 to infuse the conduit with a lubricating liquid after the insertion, in which tip is configured to attached to a special myringotomy tool. In some embodiments, a non-infused conduit is inserted into the tympanic membrane and infused after insertion by the reservoir. In other embodiments, a similar design can be introduced for other shape-changing stimuli (the effect of light, temperature, pressure, an electric or magnetic field, or a chemical stimulus). In any of the preceding and subsequent embodiments, the form of the conduit is a flat, curved, round, tubular, sharpened, mesh, or roughened surfaces of conduit, catheter, cable, or wire.

C. Tympanostomy Conduits with Anisotropic Mechanical Properties

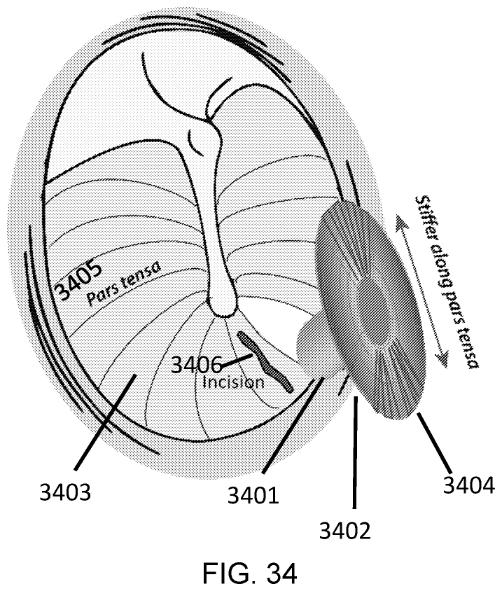

As shown, for example, in FIG. 34, the tympanic membrane has a circular/radial fibrous collagen architecture in the lamina propria that is important for allowing sound conduction to the ossicular chain at both low and high frequencies. In certain embodiments, the conduit 3401 incorporates a flange 3402 with radial stiffness that matches the portion of the tympanic membrane 3403 that is perforated to allow for more efficient sound conduction. In certain embodiments, the stiffness is imparted through additional stiffening fibers 3404 pointing in the desired direction that matches the direction fibers in the tympanic membrane (i.e. along the pars tensa 3405) or by using a mechanically anisotropic material or composite material. Non-limiting examples of fibers collagen, polyurethane, silicones, polyesters, polycarbonates, or polyethers. In certain embodiments, the flange can be made either from a nonbiodegradable material and be removed with the conduit, and in other embodiments it can be made from a biodegradable material that incorporates into the tympanic membrane and encourages cells to remodel it into tissue with a similar architecture to repair the perforation or incision 3406. FIG. 34 depicts an example of a tympanostomy conduit with flange stiffness matching the section of the tympanic membrane in which it is being placed.

D. Tympanostomy Conduits with Controllable Extrusion.

Grommet-type tympanostomy tubes tend to extrude between 9 and 18 months after insertion. Tympanic membrane epithelial migration can produce a more or less orderly sequence of events including 1) accumulation of squamous debris under the outer tube flange, 2) elevation and rotation of the tube, 3) extrusion of the inner flange, 4) closure of the tympanic perforation, and 5) outward migration of the tube with cerumen. In ˜20% of children, this does not occur. Some tubes remain in place despite the accumulation of surrounding squamous debris for years. Tubes that can remain in place can result in persistent conductive hearing loss, infection, or tympanic membrane perforation. Further, tubes that remain in place in children can result in the need to return to the operating room for removal, adding risks associated with general anesthesia.

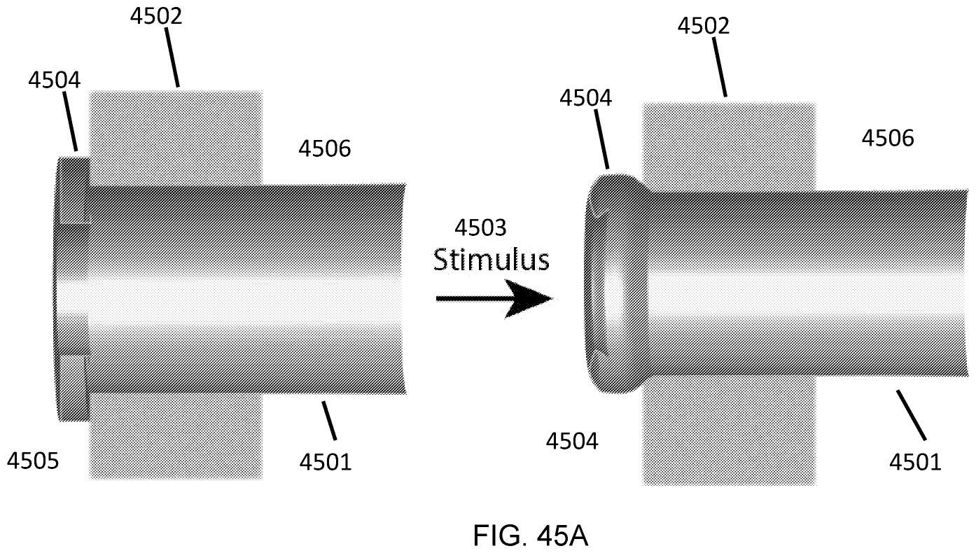

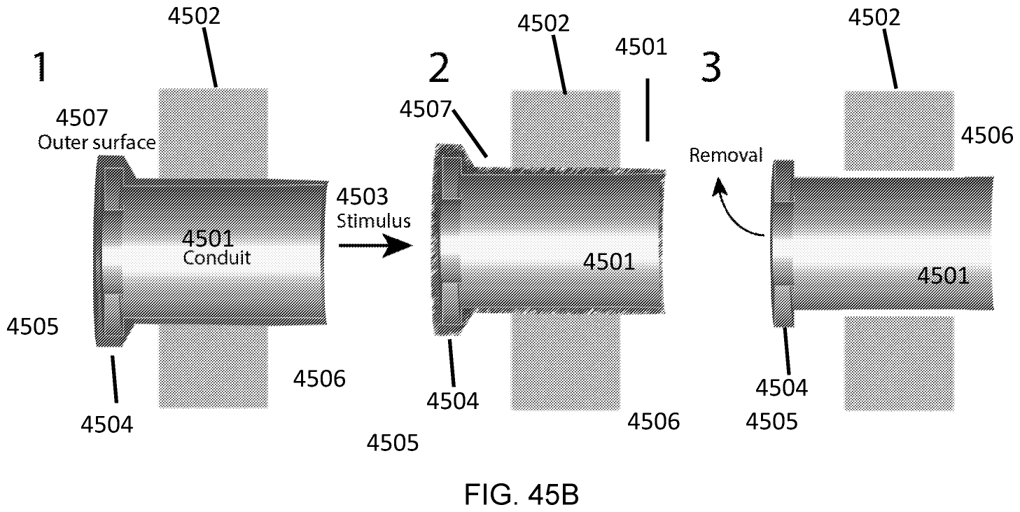

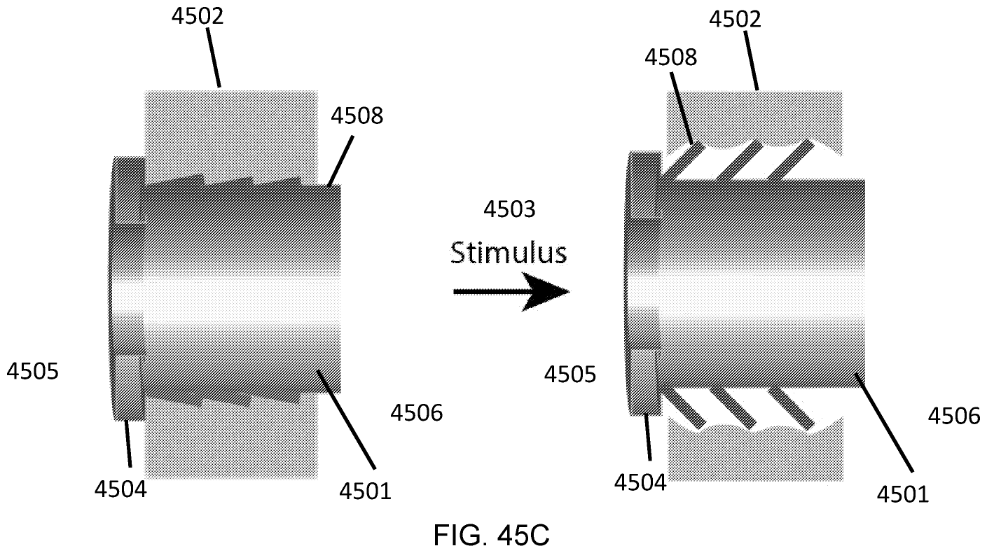

In certain embodiments, shown in FIGS. 45A-C, the stimuli-responsive or shape-change material can enable controlled extrusion of the tubes. In some embodiments, the solutions can use an excessive lubricating liquid or a liquid, or a stimulus (temperature, pH, light, electric and magnetic fields, swelling, de-swelling, and others) that either makes the flanges slippery or makes the flanges twist, roll, collapse, or induces appearance of periodic or aperiodic arrays of features (wells, bumps, holes, etc) to enable controlled extrusion of the tubes 4501 from the membrane 4502 at a desired time point, as shown in the FIG. 45A-C. In certain embodiments, shown in FIG. 45A, a stimulus 4503 causes the flanges 4504 on the side of the middle ear 4505 to collapse to enable controlled extrusion and removal through the outer ear 4506. In certain embodiments, controlled extrusion is enabled by inducing a size or shape transformation of the outer surface of the conduit, or peeling off/dissolving an external thin layer 4507 around the conduit as shown in the example of FIG. 45B. In such embodiments, the outer surface of the conduit can be coated in a thin layer of a stimuli-responsive material that separates from the outer surface of the conduit by dissolving or peeling off in response to an external stimuli. In this embodiment, when the layer separates from the outer surface, a gap remains between the outer surface of the conduit and the tympanic membrane, enabling controlled extrusion.

In some embodiments, actuators 4508 formed of shape-changing material can also be placed on the outer surface of a conduit for a built-in control of the conduit extrusion process from the tympanic membrane through external stimuli. For example, the actuators can expand or collapse, or undergo another type of size/shape and/or chemical transformation to induce the extrusion from the membrane as shown in the FIG. 45C. In the embodiment shown in FIG. 45C, the actuators expand when exposed to a stimulus, pushing away from the tympanic membrane to form a gap between the outer surface and the tympanic membrane, enabling controlled extrusion.

In some embodiments, passive extrusion can take place whereby the grommet extrudes following de-swelling of one or more components on the device. This mechanism can control the extrusion by discontinuing administration of the lubricant or other liquid. As the lubricant or other liquid seeps from the device into surrounding materials and tissues, the swollen components can gradually de-swell until the device is loosened from the hole through which the conduit is placed, allowing it to fall out or be easily removed. To speed up this process, another liquid can be placed on the tube that displaces the original lubricant and rapidly evaporates or leaves the tube, allowing the material to be de-swelled. In this manner, a patient or provider will be able to control extrusion time by controlling gradual or controlled de-swelling of the implant.

E. Tympanostomy Conduits with Sensing Components

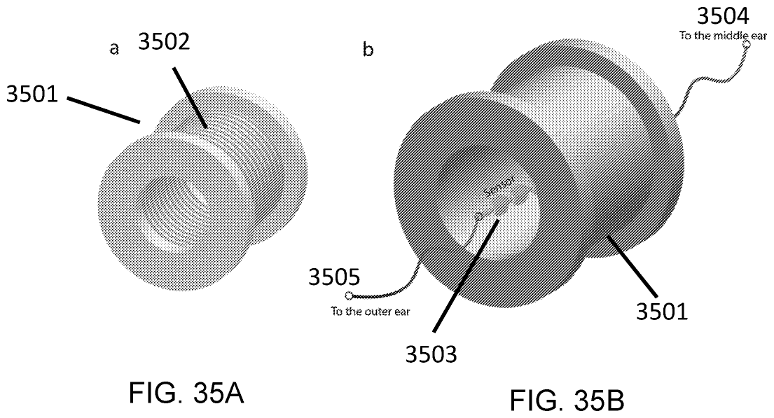

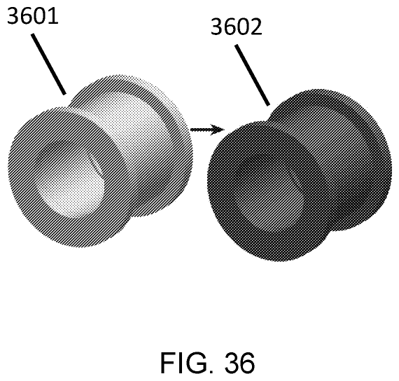

In certain embodiments, shown in FIGS. 35A-35B, relevant bodily biomarkers including at least one of temperature, moisture level, pH, pressure difference, osmolarity, drug concentration, surfactants, viscosity of the fluid and others can be introduced into the conduit 3501 via built-in antennas and sensors. FIG. 35A depicts an example of a conduit with a tunable printed antenna 3502 for sensing temperature, pH and pressure changes. In this embodiment, wires can be printed onto the conduit via lithography. FIG. 35B depicts an example of a conduit with a built-in sensor 3503 for monitoring changes in the middle ear 3504 and/or the outer ear 3505 including, for example, temperature, pH and/or pressure changes. In certain embodiments, a tympanostomy conduit that changes color upon exposure to certain stimuli conduit has colorimetric indicators based on halochromic, chromogenic photonic-crystal materials. FIG. 36 depicts a tympanostomy conduit that changes from a first color 3601 to a second color 3602 upon exposure to certain stimuli. In some embodiments, the conduit changes color when exposed to a biomarker or infectious agent. In certain embodiments, color change can indicate that the patient should have topical antibiotics applied. In certain embodiments, the conduit could change color to indicate an improvement in a patient's condition, via normal levels of biomarkers, to demonstrate that the tube can be removed.

In certain embodiments, the conduit with an antenna collects data from the patient tracking at least one of various relevant bodily biomarkers: temperature, moisture level, osmolarity, pH, pressure difference, drug concentration, surfactants, viscosity of the fluid and others that will allow for a remote monitoring of child's condition, and transfer the results to a computer or a mobile device or a wearable health tracking device. In some embodiments, the conduit can do so via antenna 3502 and/or sensor 3503.

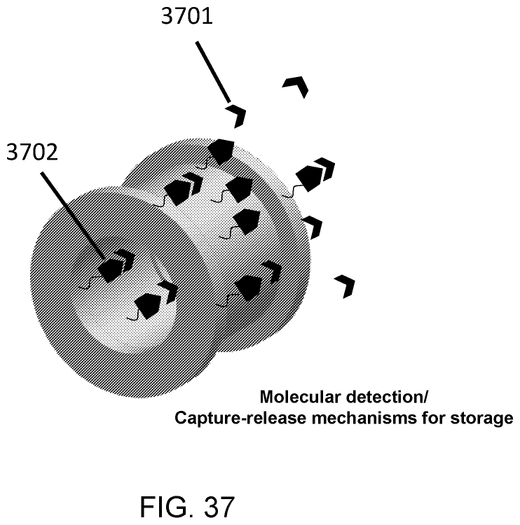

In certain embodiments, shown in FIG. 37, the conduit is capable of molecular detection of biomarkers 3701 relevant to monitoring the disease (mucus, effusion, cytokines, bacterial endo- and exotoxins, Eosinophil cationic protein, antibodies, aptamers, nanoparticles, lipases, esterases, proteases, growth factors, histamine, hormones, cytoplasm of apoptotic cells, macrophages or other immune cells, blood, or external pollutants, e. g. diesel exhaust particles and other air pollutants. These biomarkers can be captured on the on the surface or within the matrix of the conduit, by capture elements 3702 for further on-demand release. In certain embodiments, the biomarkers can be captured or immobilized on the surface or within the matrix based on specific interactions between the capture element and the biomarker. When exposed to a stimulus, the capture elements can release the biomarkers, for example as the result of a conformational change or a disruption of the interaction between the biomarker and the capture element. In certain embodiments, capture elements by antibodies, aptamers, nanoparticles, lipases, esterases, proteases, growth factors, histamine, hormones, cytoplasm of apoptotic cells, macrophages or other immune cells, blood, pH, salt levels, temperature.

In certain embodiments, the conduit undergoes on-demand enabled shape and chemistry transformations for temporary point-of-care applications where the local or “as a whole” transformation takes place for limited or unlimited amount of time for enhancing, reducing, redirecting or blocking liquid transport (for example, for drug delivery and protection of the middle and/or inner ears from external conditions) or used for the controlled extrusion purposes. While the following and above description includes certain embodiments relating to tympanostomy conduits and/or subannular ventilation conduits, the designs can be used in other medical or non-medical applications, such as microfluidic devices, membrane, bioreactors, nozzles, transport of coolant and other chemicals through machinery, drainage of waste products from reactions, sensors, food and beverage industry, cosmetics and perfumes, and other applications. porous networks, conjugated particles, nanotextured surfaces, or enzymes.

### VI. Tympanostomy Conduits as Medical Devices for Effective Therapeutic Delivery for Treating Ear Disorders

In certain embodiments, the conduit provides solutions for treating a number of middle and inner ear diseases and disorders. In certain embodiments, a conduit is specifically designed to enable an efficacious “first-in-class” drug delivery and thereby decrease time of treatment and morbidity, and direct and indirect costs associated with failed treatment.

A. Tympanostomy Conduits Guiding Therapeutics into the Middle Ear

A number of ear diseases can be treated with topical therapeutics, including bacterial infections, sensorineural hearing loss, and Meniere's disease. Characteristic of topical delivery systems is the absence of systemic effects, which is an advantage if no systemic effect is required. For example, systemic administration of antibiotics for otitis media can result Clostridium difficile (C. diff) infections and antibiotic resistant organisms, such as Methicillin-resistant Staphylococcus aureus (MRSA). Systemic steroids for sensorineural hearing loss has a host of significant side effects, ranging from anxiety and reflux, to avascular necrosis of the hip and psychosis. Systemic reaction to topical antibiotics and steroids is extremely uncommon. Further, the use of topical agents allows for the simultaneous modification of the local microenvironment. The pH of the external auditory canal, for example, is normally slightly acidic. The administration of an antibiotic in an acidic drop helps restore and fortify this normal host defense mechanism. Ototopical medications are generally less expensive than systemic medications.

Another example of an ear disease that would benefit from the topical drug administration is the Meniere's disease, which is treated with gentamicin and steroids. For example, the gentamicin and/or steroids can be injected into the tympanum, or middle ear, through the ear drum. This can be done with a minor surgical procedure performed in the office. Gentamicin is used in patients to stop attacks of vertigo. It is a medication which is toxic to the inner ear but is more toxic to the vestibular cells than the hearing cells of the inner ear. This can allow elimination of enough vestibular cells to stop vertigo attacks without a significant change in hearing.

Placement of a short- or long-term tympanostomy conduit with designs can decrease the need for repeated procedures. Indeed, in some patients, a tympanostomy conduit placed in to the eardrum can replace the intratympanic injection, instead, the medication is injected through the conduit or the patient can self-treat with drops at home. A number of therapeutics can be delivered more efficiently through the conduit disclosed herein, by means of non-limiting example, including: antibiotics, antiseptics, anti-viral agents, anti-inflammatory agents, small molecules, immunologics, nanoparticles, genetic therapies including viral and lipid based therapies, chemotherapeutics, stem cells, cellular therapeutics, growth factors, proteins, radioactive materials, or other liquid and gas-based pharmaceutical compounds.

In some embodiments, a conduit includes a single-, dual- or multi-channel conduit with patterned chemical properties and texture, as shown in other sections of this disclosure. In certain embodiments, different channels of conduits are optimized for the transport of topical medication into the middle ear (for example, as shown in FIG. 21B). In certain embodiments, these channels are combined with or without flanges and, in certain embodiments, can have a conical geometry to specify the flow into the middle ear. Flanges can also be designed specifically to wick the ototopical drops into the tympanum.

In certain embodiments, a conduit includes porous material within the lumen representing a) an array of channels, or three-dimensional b) periodic or a) aperiodic (sponge-like) interconnected network of pores of sizes ranging from 0.01 to 1000 μm, with specific chemical modification of the pores allowing for selective therapeutic delivery into the tympanum. The tailored surface functionalities can include: perfluorooctyltrichlorosilane triethoxsilylbutyraldehyde, bis(2-hydroxyethyl)-3-aminopropyltriethoxysilane, 3 chloropropyltriethoxysilane, 3-(trihydroxysilyl)-1-propanesulfonic acid, n-(triethoxysilylpropyl)-alpha-poly-ethylene oxide urethane, n-(trimethoxysilylpropyl)ethylene diamine triacetic acid, n-octyltriethoxysilane, n-octadecyltriethoxysilane, (3-trimethoxysilylpropyl)diethylenetriamine, methyltriethoxysilane, hexyltrimethoxysilane, 3-aminopropyltriethoxysilane, hexadecyltriethoxysilane 3-mercaptopropyltrimethoxysilane, dodecyltriethoxysilane; or chiral functionalities, such as N-(3-Triethoxysilylpropyl)gluconamide or (R)-N-Triethoxysilylpropyl-O-Quinineurethane).

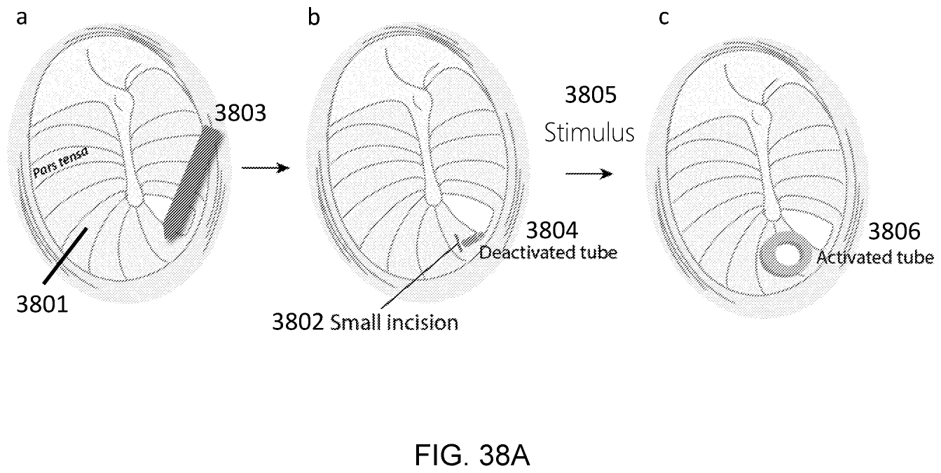

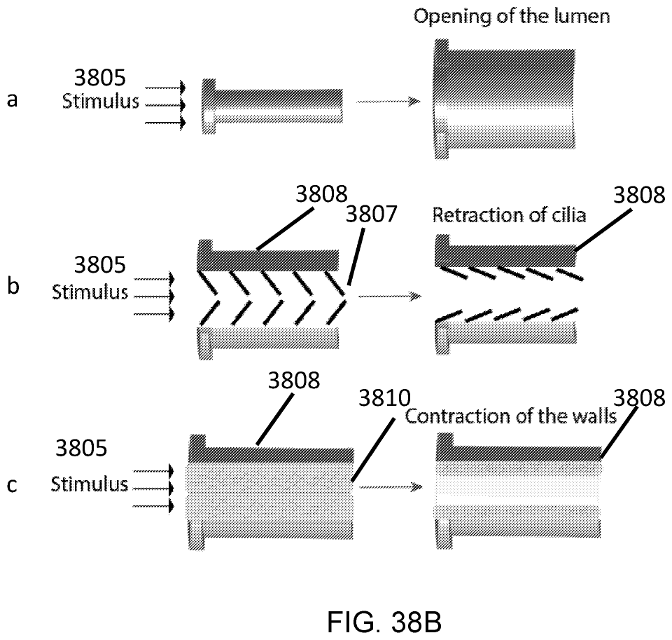

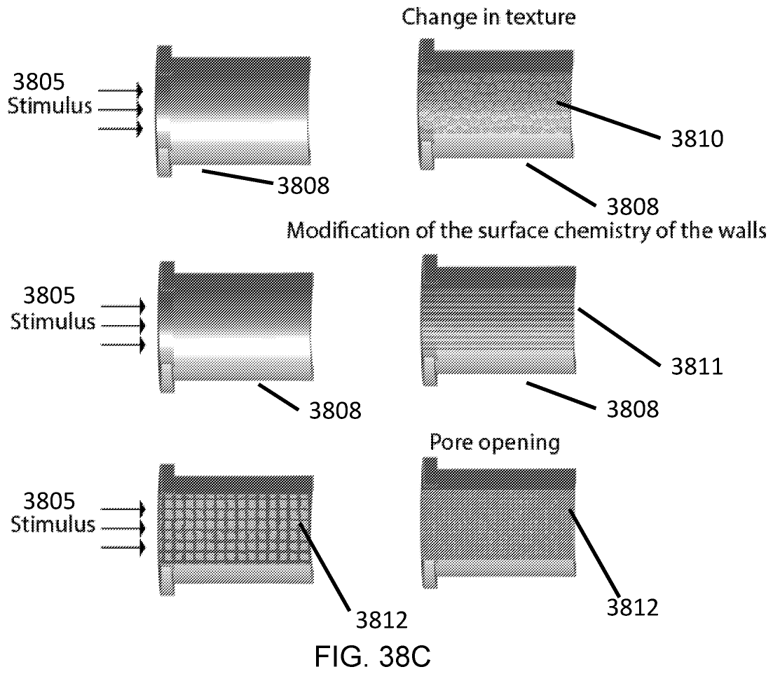

In certain embodiments, shown in FIG. 38, programmable conduits are inserted in the tympanic membrane 3801. In some embodiments, a small incision 3802 is created in the tympanic membrane by a surgical tool 3803. A deactivated tube 3804 with a small size can be inserted into the incision. The deactivated tube can be closed such that fluid cannot pass through the lumen of the deactivated tube. In response to a stimulus 3805, tube can transform into an activated tube 3806 such that fluid can pass through the lumen of the tube. In certain embodiments, conduits are dynamic and/or programmable such that they can be reversibly actuated on demand to facilitate the delivery into the middle ear through a temporary or long-term opening of the lumen via expansion of the conduit radius (see, e.g., FIG. 38B view a), and/or change in the texture, surface chemical properties, micro- and macro-structured stimuli-responsive cilia-like and hair-like fibers 3807, platelets, pillars and other architectures on the inside of walls 3808 of the conduit, as shown in the FIG. 38B (view b). For example, the cilia can retract to open the lumen and allow fluid to pass through the lumen. In certain embodiments, the walls of the lumen can be coated with a material 3809 that contracts when stimulated (FIG. 38B view c). In certain embodiments, texture 3810 of the walls of the tube can change in response to the stimulus to open the lumen. In this embodiment, the fluid can be unable to pass through the lumen when the tube is first inserted but able to pass through the lumen after the texture change. Non-limiting examples of texture change include increased roughness, decreased roughness, formation of grooves, formation of raised structures, formation of depressed structures, texture due to texture agent additives, e.g. micron-sized particles (in the range between 1 and 1000 μm,

In certain embodiments, the surface chemistry 3811 of the walls can change in response to the stimulus to open the lumen. In this embodiment, the fluid can be unable to pass through the lumen when the tube is first inserted but able to pass through the lumen after the surface chemistry change. Non limiting examples of surface chemistry change include hydrophobicity, hydrophilicity, omniphobicity or peptide or polymer conjugation. In certain embodiments, shown in FIG. 38C (view c), the lumen can contain a material with pores 3812 that are closed when the tube is inserted. The pores can open in response to a stimulus 3805. Porous materials within the lumen are described with respect to FIG. 21. In these embodiments, the conduit is placed into the tympanic membrane in a closed, deactivated state, and is activated on demand for the drug delivery or other relevant medical treatment though one or more stimuli: the effect of light, temperature, pressure, an electric or magnetic field, or a chemical stimulus, pH, light, swelling, de-swelling, humidity, electron transfer, or other as exemplified in FIGS. 38B-38C.

In certain embodiments, the stimuli-responsive materials can be of thermostrictive, piezoelectric, electroactive, chemostrictive, magnetostrictive, photosensitive and photostrictive, or pH-sensitive nature. These materials can utilize light-driven therapeutic cargo control, where UV light triggers cargo flow through the conduit. In certain embodiments, materials can utilize controlled electric conduction. In certain embodiments. the top layer of the liquid medium is conductive, or the liquid medium has a solid conductive confining surface on the top of device. In other embodiments, the tips of microstructures are also modified with conductive materials. In certain embodiments using electrical conduction, the electric conduction of the surface or the whole system can be controlled by chemically-induced mechanical actuation of the microstructures.

In certain embodiments, the self-modulated adaptively reconfigurable tunable nano- or microstructures with appropriately functionalized (chemically or physically) tips embedded in a hydrogel, as described in U.S. Pat. No. 9,651,548 “Self-regulating chemo-mechano-chemical systems” issued on May 16, 2017, which is incorporated herein by reference. This dynamic system incorporates the movement of “skeletal” high-aspect-ratio microstructures (posts, blades, etc.) by a polymeric “muscle” provided by the swelling/contracting capabilities of the hydrogel in which the microstructures are embedded. In certain embodiments, the layers are arranged vertically, one stacked over the other. In certain embodiments, the system can be also designed horizontally with these two layers positioned side-to-side.

B. Tympanostomy Conduits with Vascular Networks for Drug Delivery to the Tympanic Membrane Surface

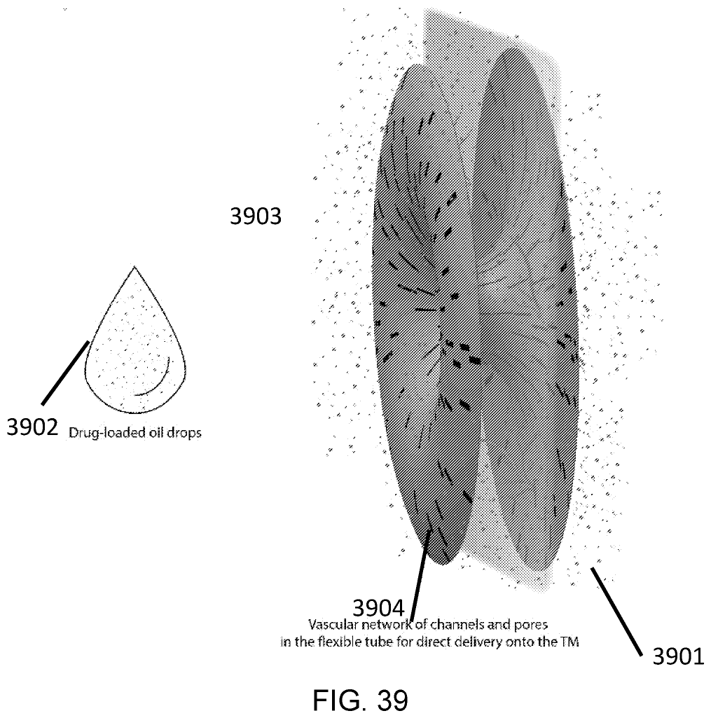

OM can present itself either as an infection inside the middle ear space due to a buildup of fluid or as an infection on tympanic membrane itself. In certain embodiments, shown in FIG. 39, the tympanostomy conduit allows for preferential drug delivery to either the tympanic membrane 3901 surface or the middle ear space, depending on the droplets 3902 used. For example, in certain embodiments the tympanostomy conduit 3903 incorporates a vascular network 3904 within its walls (see, e.g., FIG. 39), for example, from a fugitive porogen or patterned channels, that allows antibiotic droplets to travel throughout the vascular network due to capillary forces, landing on the surface of the tympanic membrane. In certain embodiments, the droplets diffuse out of the vascular network and onto the tympanic membrane. In certain embodiments, the droplets are designed to match the material properties of the tympanostomy conduit to allow for better adhesion. For example, in certain embodiments a liquid infused tympanostomy conduit has droplets encapsulated in the same liquid (for example, oil). In some embodiments, it is desired for the droplets to enter the middle ear space instead of the surface of the tympanic membrane the droplets are made from a different liquid, such as surfactant-filled aqueous solution, which experiences difficulty in entering these channels. In other embodiments, the droplets are encapsulated in microparticles that cannot fit into these microchannels and thus can only travel through the main lumen in the center. In certain embodiments, these microparticles can be made of any biodegradable polymer.

A number of therapeutics can be delivered efficiently through the vascular network, including, but not limited to antibiotics, antiseptics, anti-viral agents, anti-inflammatory agents, small molecules, immunologics, nanoparticles, genetic therapies including viral and lipid based therapies, chemotherapeutics, stem cells, cellular therapeutics, growth factors, proteins, radioactive materials, and other liquid and gas-based pharmaceutical compounds.

C. Drug Delivery Though the Lubricant Overlayer

Certain embodiments relate to a medical device for delivering a therapeutic agent to the body tissue of a patient, and methods for using such a medical device. For example, in some embodiments, drug eluting tubes incorporate synthetic slippery lubricant-infused surfaces for repelling fluids of biological origin while allowing for effective drug release from the tube. In certain embodiments, Drugs to be included in the drug eluting tubes disclosed herein can either be incorporated in the solid matrix supporting the entrapped liquid or other liquid-like matrix and then diffuse over time through the lubricating liquid layer into the surrounding tissue or the drugs can be incorporated within the lubricating liquid layer and then diffuse into the surrounding tissue. In accordance with certain embodiments, the drugs can be incorporated in both the solid matrix and the lubricating liquid layer. In certain embodiments, drugs used in these applications can be either extremely hydrophobic or hydrophilic and can be difficult to dissolve in the lubricating liquid layer. Therefore, even if drugs can be introduced into the underlying solid substrate, the drugs cannot be able to diffuse through the lubricating liquid layer and will remain trapped. Lubricants useful in the embodiments related to delivery though the lubricant overlayer should allow for sufficiently low surface energy while allowing for effective drug release from the tube. Non-limiting examples of an entrapped liquid include oils, hydrogels, organogels, or reconfigurable molecules possessing highly flexible long chains such as long polydimethylsiloxane polymers or other types of polymers and copolymers, including random or block silicone co-polymers with other siloxane co-monomers featuring alkyl, aryl, aralkyl substituents on silicon atoms that can be grafted to a solid surface.

A range of surface structures with different feature sizes and porosities can be used. Feature sizes can be in the range of tens of nanometers to microns (e.g., 10 to 1000 nm), and have aspect ratios from about 1:1 to 10:1. In certain embodiments, the surface has a large surface area that is readily wetted by the lubricating liquid and which entrains lubricating liquid and retains it on the substrate surface.

In certain embodiments, ore than one drug or biologically active component can be used in accordance with certain aspects. The compounds can be released from the lubricating layer by diffusion, degradation or other mechanism or combination of mechanisms, which provide for the desired release profile. Other suitable drugs, therapeutic materials, etc. for including in stents are disclosed in U.S. Pat. No. 8,147,539 to McMorrow et al., issued on Apr. 3, 2012, the contents of which are hereby incorporated by reference.

In certain embodiments, drugs can be incorporated into the lubricating layer, the solid matrix supporting the entrapped liquid, or any combination thereof. Drug eluting stents can be prepared by mixing the drug with the polymer melt and then casting the melt to form the stent, according to certain embodiments. The drugs can also be encapsulated in particles or micelles and then dispersed in an oil in certain embodiments. Examples of such dispersions of encapsulated drugs include forming complexes with cyclodextrin and oil to create these particles. In certain embodiments, the drug can also be encapsulated in carriers made of lipid molecules, block co-polymers or both. In certain embodiments, the drug can also be encapsulated in particle carriers made of lipid molecules, polymers, or a combination or both and these particles can be added into the drug suspension that is applied to the outer lumen of the tube.

The following example further describes and demonstrates embodiments within the scope of the present invention. The examples are given solely for the purpose of illustration and are not to be construed as limitations of the present invention, as many variations thereof are possible without departing from the spirit and scope of the invention.

In certain embodiments, release of drug is intermediate, and the profile can be tuned by reducing the drug loading in the lubricating liquid layer and tuning the lubricating liquid layer thickness. If slow drug release over the course of a few months is desired, one possibility is to load the underlying substrate with the drug and have the drug diffuse slowly through the lubricating liquid layer over time. In one non-limiting example, the drug is paclitaxel and the lubricating liquid layer is castor oil. If the lubricating liquid layer depletes over time, the drug can also possibly be released from the substrate of the conduit after this depletion takes place. Many parameters can be tuned to achieve a desired release profile. For example, the following parameters can be taken into consideration to develop a certain drug release profile: Oil layer thickness, oil layer viscosity, drug concentration within the oil layer, surface area of tube coated with the oil layer, drug concentration within the porous matrix/substrate, and material used for porous matrix/substrate.

D. Tympanostomy Conduits for Drug Delivery to the Inner Ear

The round window (RW) and oval window (OW) are two openings from the middle ear into the inner ear, including cochlea. The round window membrane (RWM) and oval window membrane (OWM), vibrate with acoustic energy transmitted from the tympanic membrane to the ossicular change, allowing conversion of mechanical energy to electrical neuronal potentials at the level of hair cells in the inner ear. Given anatomic location, the RWM can be a site for drug delivery to the inner ear. The RWM can be used as the site of cochlear implantation. The RWM can act as a barrier to ototoxic substances in the middle ear and participate in the secretion and absorption of substances. Animal experiments show that the RWM behaves like a semipermeable membrane. Many substances with both low and high molecular weights have been demonstrated to penetrate through the RWM when placed in the round window niche. These substances include sodium ions, antibiotics, antiseptics, arachidonic acid metabolites, local anesthetics, toxins and albumin. The permeability of the RWM can be influenced by the factors such as size, configuration, concentration, liposolubility and electrical charge of the substance, and the thickness and the condition of the RWM.

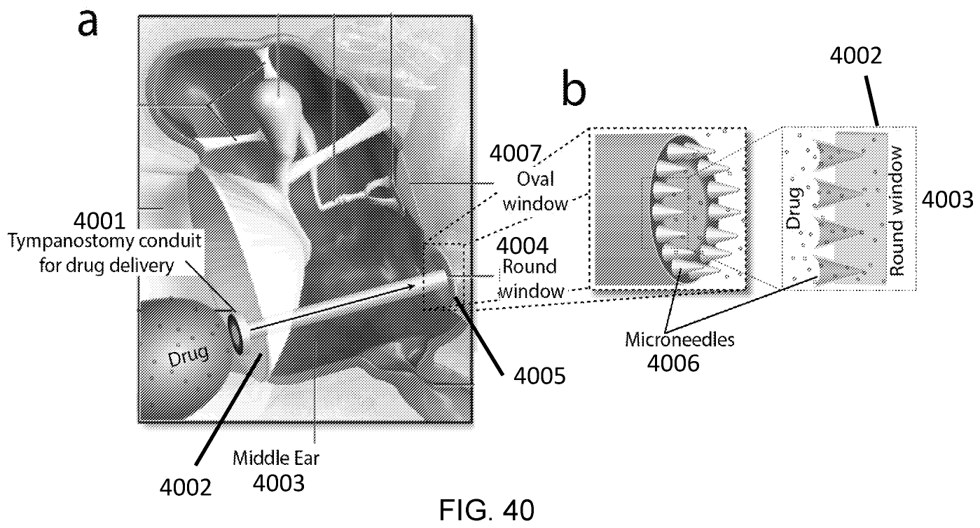

FIG. 40 shows an embodiment of the invention where a conduit 4001 is placed through an opening in the tympanic membrane 4002 into the middle ear 4003 and extends across the middle ear to the surface of the round window 4004. As the round window is generally impermeable to most small molecules and growth factors that could be used as therapeutics in hair cell regeneration, these molecules could be transferred via a carrier solution in accordance with certain embodiments. In certain embodiments, a tube can be placed near an entrance to the semicircular for the delivery of therapeutics to aid in balance disorders. In certain embodiments, tubes can be designed such that the perilymph or endolymph of the inner ear cannot exit, while the drug solution can enter the tube. In certain embodiments, the tube could be designed such that the perilymph or endolymph can exit above a certain pressure value, allowing for equalization of pressure in the cochlea and to prevent over pressurization following the delivery of medications.

In certain embodiments, the distal end 4005 of this conduit can either rest near the tissue, be chemically attached to the tissue via an adhesive agent, or be mechanically attached to the tissue via mechanisms including at least one hook, macro-needle, or micro-needle 4006 to enable drug delivery into the inner ear via the round window. In certain embodiments, such mechanisms, as shown in FIG. 40, can be composed of biodegradable and/or non-biodegradable materials, according to certain embodiments. These mechanisms can be used to anchor the distal end of the implant in place or to guide the therapeutic into the region of interest via capillary action, diffusion, or externally applied pressure at the proximal end of the conduit, according to certain embodiments. This design can be used to deliver therapeutic agents, such as steroids, antibiotics, antivirals, growth factors, small molecules, proteins, gene therapy agents, chemotherapeutics, radioactive substances, nanoparticles, cellular therapy agents. In certain embodiments, this design can deliver a growth factors to restore functionality in cochlear hair cells to restore hearing in patients with hearing loss. In certain embodiments, the distal end of the conduit is attached to or near the oval window 4007 or other component of the cochlea, semi-circular canals of the vestibular system or the bloodstream. In other embodiments, the distal end of the conduit rests within the middle ear space to deliver therapeutic agents transtympanically. The therapeutic delivery can be facilitated by (i) solid microneedles for skin pretreatment to increase skin permeability, (ii) microneedles coated with drug that dissolves off in the skin, (iii) polymer microneedles that encapsulate drug and fully dissolve in the skin, and/or (iv) hollow microneedles for drug infusion into the skin, according to certain embodiments.

In certain embodiments, the interaction of an administered drug-containing solution with the lubricating liquid layer or physical structure of the implant can cause a physically or chemically-induced phase transition of the solution. In some embodiments, mechanisms could be used to increase the viscosity of the solution to remain within the middle ear space. Non-limiting examples of such mechanisms include foaming, gelation, or increased cross-linking. These mechanisms can be useful to prevent the solution from leaking through the Eustachian tubes or back out of the tympanostomy tube after it traverses the tympanic membrane.

In certain embodiments, the lumen could contain a porous network that introduces a phase into the liquid to produce a foam-like composition in a physically-induced phase transition. In other embodiments, surface features on the lumen surface could cause turbulent mixing of the solution with air, producing a foam-like composition. Surfactants could be incorporated into the administered solution or the lubricant overlayer to aid in stabilization of the air bubbles within these foams.

In certain embodiments, molecular organogelators convert oils into gels by forming self-assembled fibrous networks in a chemically-induced phase transition. In certain embodiments, gelation can be activated by contacting the oil with an immiscible solvent (water). Synthetic small-molecules known as organogelators have the ability to self-assemble into long fibers when introduced into organic liquids (oils). These fibers entangle and interconnect into a three-dimensional (3-D) network, thereby converting the oil into an elastic organogel. Gelation can be achieved in response to external stimuli or environments such as temperature, redox states, pH, ultrasound, or light. Upon irradiation with light, the gelator can be photoisomerized, whereupon it becomes an active gelator. Thus, light can be used as a “switch” to activate the gelator, according to certain embodiments. In other embodiments, the lubricant could contain a crosslinking mechanism introduces covalent, ionic, van der Waals, or other increased interactions between molecules in the solution. Non-limiting examples of a crosslinking mechanism include calcium ions for an alginate solution, poly(2-hydroxyethyl methacrylate) crosslinking, hydrogen bonding of phospholipid polymers, alkyne-azide click reactions.

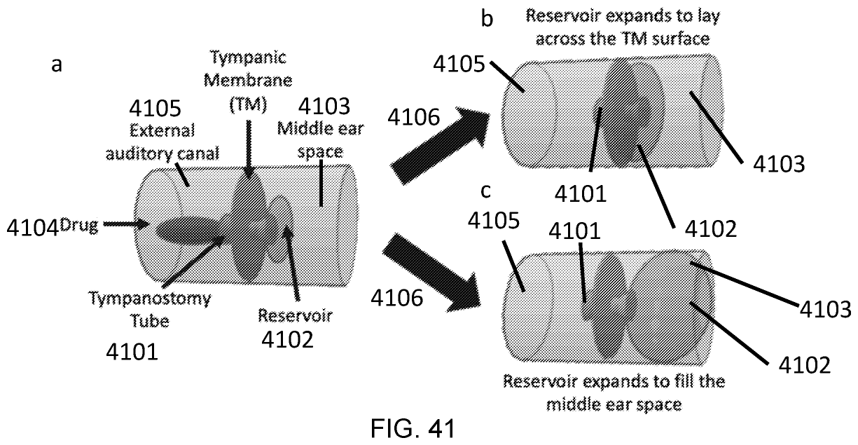

In certain embodiments, shown in FIG. 41, the tube 4101 comprises an expandable reservoir 4102 on the middle ear side 4103 of the tube, as shown in FIG. 41 (view a). Non-limiting examples of a reservoir include a porous polymer, a hydrogel, or a balloon-like structure. When a therapeutic 4104 (such as an antibiotic, a steroid, or another drug) is introduced through the ear canal side or external auditory canal 4105 of the tube, the therapeutic can travel through the tube collect in the reservoir. The reservoir then absorbs the therapeutic. In certain embodiments, the reservoir can expand to cover the surface of the tympanic membrane (FIG. 41 view b) or to touch certain parts of the middle ear space (FIG. 41 view c). In some embodiments, the reservoir expands in response to a stimulus 4106. In some embodiments, the reservoir could be designed to allow the therapeutic to pass through the surface of the reservoir onto the tympanic membrane surface, middle ear interior surface, the ossicles, the round window surface, or oval window surface. In this embodiment, contact with these structures or tissues allows for targeted delivery of the therapeutic. In other embodiments, the reservoir could be designed to generally elute the therapeutic into the middle ear space. In some embodiments, additional fluids or pressure could be added at the ear canal side of the tube to promote or increase the rate of elution of the therapeutic from the reservoir.

E. Tympanostomy Conduits with Pinning to Reduce and/or Prevent Environmental Water Entrance

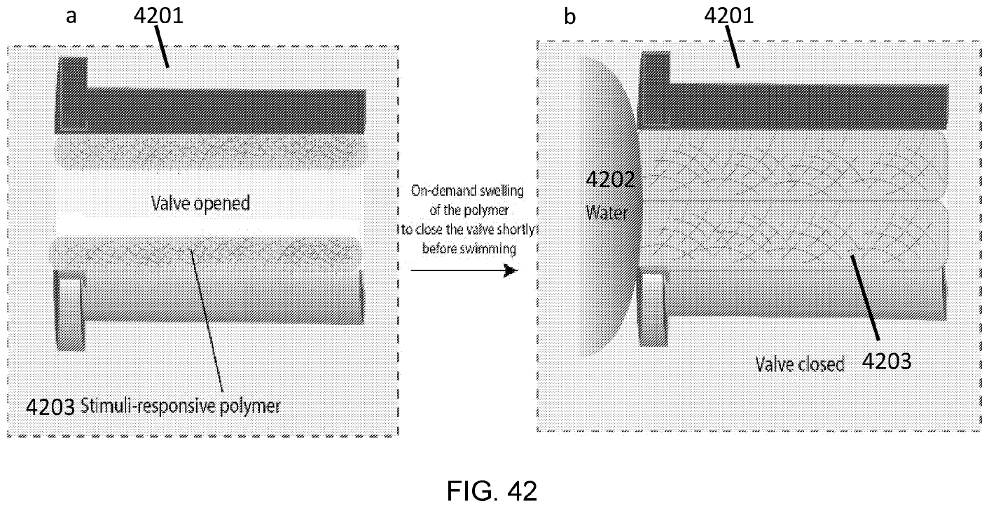

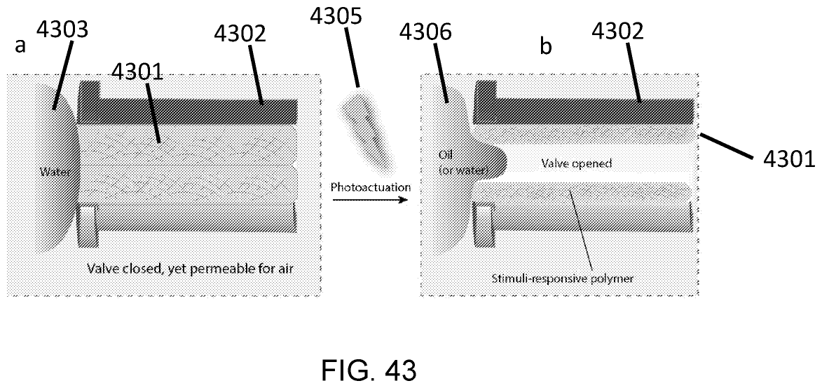

In certain embodiments, the lumen of the tympanostomy conduit can be gated by another material that allows for transport of certain fluids or fluids under certain conditions into the conduit while keeping out other fluids. In certain embodiments, shown in FIG. 42, the lumen of the conduit can be open in response to a stimulus to allow delivery of a therapeutic, for example antibiotic droplets, at a specific time. For example, one can desire for antibiotic droplets to enter the conduit 4201 but normal water 4202 to come out. In certain embodiments, the liquid can be propelled by capillary force arising from photo-induced asymmetric deformation (e.g. in liquid crystalline elastomers), wettability gradients, or the Marangoni effect. FIG. 42 shows such a lumen with swelling controlled by the deposition of specially designed droplets, according to certain embodiments. In certain embodiments, this swelling occurs due to droplets containing ionic crosslinkers or a fluid that can be absorbed by the stimuli responsive polymer 4203 such as a crosslinked polymer or hydrogel lining the lumen. This swelling closes off the channel from water penetration, as shown in FIG. 42 (view b) until either the swelling wears off or the patient inserts another type of droplet into their ear. In other embodiments, shown in FIG. 43, the lumen contains a polymer 4301 that expands (FIG. 43 view a) or contracts (FIG. 43 view b) in response to stimuli such as light or heat, for example as shown in FIG. 43. In certain embodiments, when the polymer expands, the lumen of the tube 4302 is closed and water 4303 cannot enter. In certain embodiments, when the tube is exposed to a stimulus 4305, for example light, the polymer contracts, opening the lumen and allowing oil 4306 or water to enter. In certain embodiments, photosensitive surfactants are added to the droplets to enhance the effect. In certain embodiments photoactuation is replaced by heating, ultrasound or electric field.

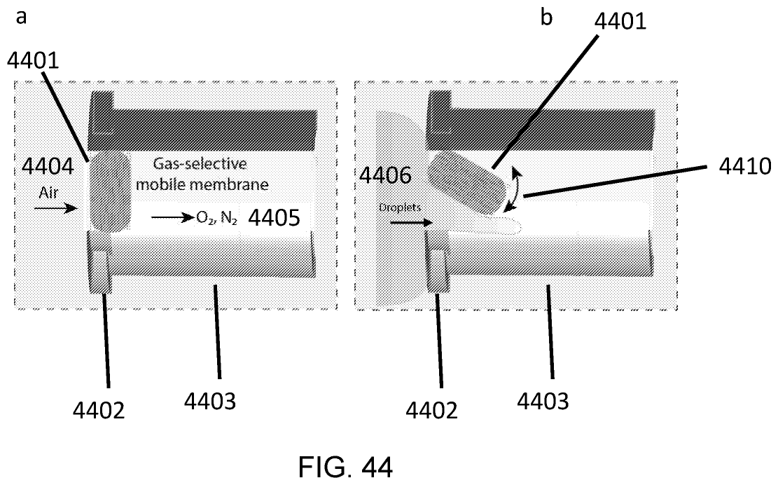

In other embodiments, the lumen of the tympanostomy conduit is gated by another material that allows for transport of certain fluid and gas exchange between the environment and middle ear space. In certain embodiments, for example shown in FIG. 44 (view a), a plug 4401 at the proximal end 4402 of the tube 4403 that allows exchange of air 4404 including oxygen and nitrogen gas 4405, with the environment but is impermeable to water (such as silicone) allows children to go swimming while still equalizing air pressure build up for cases of OM that do not require fluid drainage, as shown for example in FIG. 44. In certain embodiments, for example shown in FIG. 44 (view b), this plug has a mechanism to open and allow certain fluids to flow into or out of the conduit, either based on the type of fluid or the amount of fluid present. For example, in certain embodiments mucus leaves the conduit when a certain pressure is reached inside the conduit. In certain embodiments, antibiotic droplets 4406 enter the conduit if they contain surfactants to “loosen” the perimeter of the gate. In certain embodiments, the plug 4401 can swing in direction of arrow 4410 to allow certain fluids to flow into or out of the conduit.

### I. Examples

A. Animal Model

The chinchilla (Chinchilla lanigera) animal model is the most widely utilized animal in middle ear research due to size and anatomy of the tympanic membrane (TM). Female chinchillas Lanigera (total number of 6) were anesthetized in routine fashion to undergo auditory brainstem response (ABR) and distortion product otoacoustic emissions (DPOAE) testing. To perform ABR/DPOAE, the anesthetized animals were placed in a sound-treated booth. Needle ABR leads were placed in standard, stereotypical fashion and bilateral ABR and DPOAE thresholds were obtained at 0.5, 1, 2, 4, 8, and 16 kHz using the Eaton-Peabody Laboratories cochlear function test suite (EPL CFTS) written in LabVIEW. EPL CFTS was used to control digital stimulus generation and data acquisition utilizing the input/output boards installed on the PXI chassis. Thresholds in the same animals have been measured on separate occasions with highly reproducible values. The difference between ABR and DPOAE testing can indicate a conductive hearing loss.

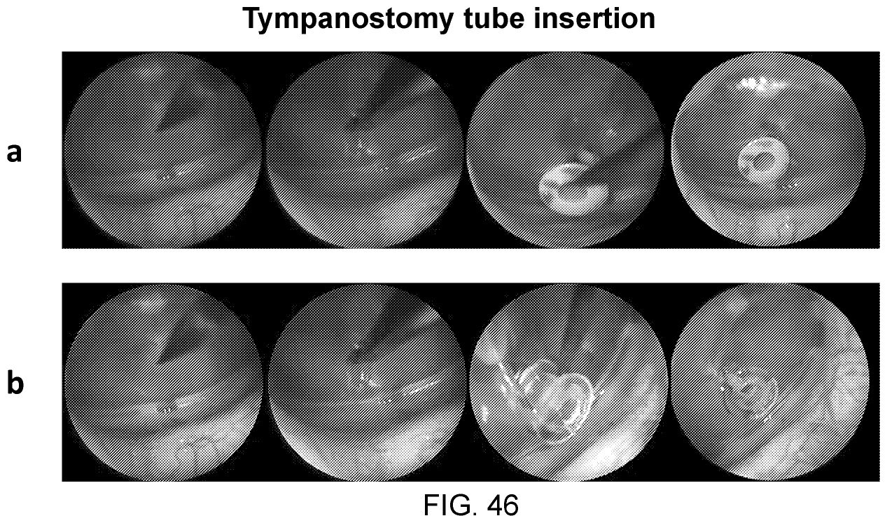

Following the ABR/DPOAE tests, tympanostomy tubes were placed into both ears in the surgical sterile facility, as shown in FIG. 46, which represents a progression of images taken of tubes being placed into ears from left to right. FIG. 46 (view a) shows placement of a Summit Medical Collar Button tube and FIG. 46 (view b) shows placement of an oil-infused silicone Collar Button tube. Using a rigid 0° and 30° Storz Hopkins® rod endoscope, the TM was visualized. Betadine was placed into the ear canal to sterilize the external auditory canal. Using a myringotomy knife, a radial 2 mm incision (myringotomy) was made on the tympanic membrane to insert the TTs. One ear received a control tube (silicone Collar Button, ID=1.27 mm, VT-1002-01, Summit Medical), the other ear—a ‘test’ tube (oil-infused silicone TTs with “H” geometry, ID=1.28±0.02 mm).

Prior to placement, all test TTs were sterilized with an autoclave at 121° C. with a 25 min wet and a 15 min dry cycle, and then exposed to ultraviolet germicidal irradiation, prior to the myringotomy procedure to insertion into the TM. After the TT placement, the animals were allowed to recover for 2 weeks, and TT were closely monitored by weekly otoendoscopy.

After the 2-week recovery period, the animal underwent a second round of general anesthesia to for ABR/DPOAE testing, as described above. After ABR/DPAOE testing, TTs were removed from the TM. For this, the ear canal was first evaluated with a 30° Storz Hopkins® rod endoscope. Then, using a sterile rosen needle, the tube was gently teased out of the prior myringotomy. Alligator forceps were used to grasp the tube gently, lift it from the ear canal under direct visualization and deliver it into a vial with PBS for further analysis. Otoendoscopic images were obtained of the TM before and after the removal of the TT. The same procedure was done on the contralateral ear. The animal was then permitted to recover for an additional 10 weeks. Photographs of the TM obtained by the endoscope were obtained with the animal awake on a weekly basis to document the healing of the perforation.

B. Evaluation of Hearing Loss

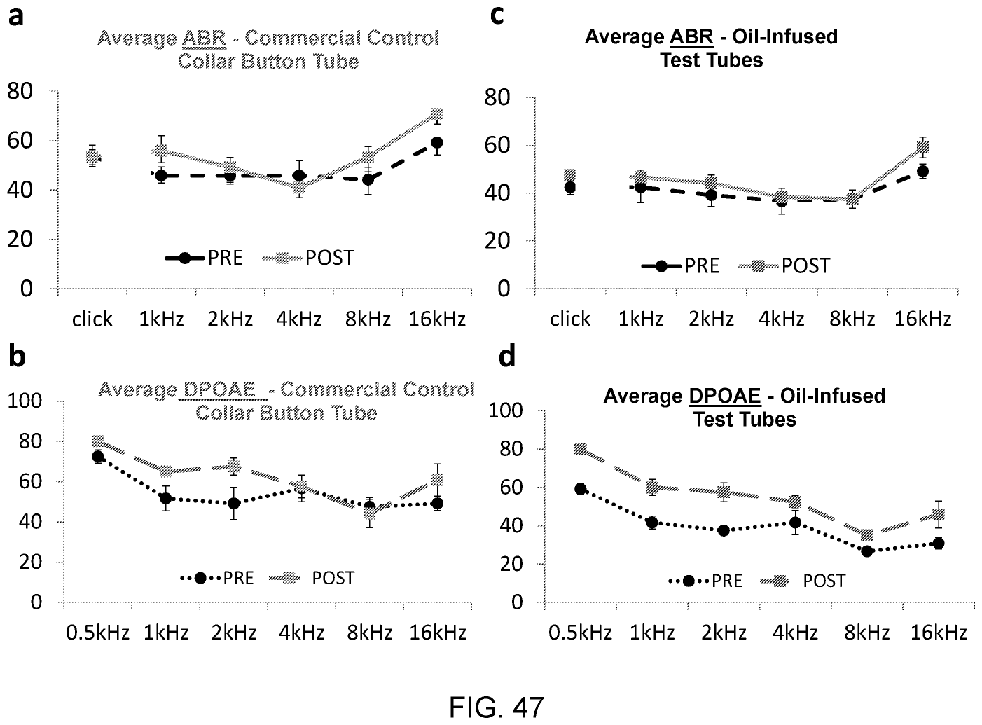

Throughout the duration of the study the observational logs did not reveal any signs of distress in any Chinchilla subjects either from the experimental group or control group. As shown in the FIG. 47, experimental tubes performed similarly with no distinguishable difference in ABR/DPOAE when directly compared with controls, and between the surgeries. In tubes explanted at 2 weeks, there were no observable differences between in vivo experimental ABR and DPOAE, confirming that the implanted tube does not cause any sensorineural hearing loss. In tubes explanted at 2 weeks, there was only slight difference in in vivo experimental DPOAE that is explained by ˜30% larger mass of the test tube.

C. Tissue Response to Tympanostomy Tubes

Ear canals hosting the control tube normally had a wet environment adjacent to the tube. The immediate area around the tube as well as some of the tympanic membrane glistened and sometimes showed mucus. The degree of inflammation visible on otoscopy was notable. Five out of six ear canals which hosted test tubes had, on the other hand, a dry environment. The degree of inflammation was visibly less in these animals. Several animals whose tympanic membrane hosted the non-oil-infused control tube had signs of inflammation or buildup around the tube, compared to animals with the implanted test tubes that were oil-infused that had no signs of inflammation or granulation. The tympanic membranes healed well around all the test tubes within 12 weeks of removal, as opposed to some of the control tubes. All control and sample tubes remained patent (unobstructed and affording free passage) when observed during extraction surgeries.

D. Bacterial Adhesion on Tympanostomy Tubes

Surgically removed TTs from chinchillas were placed in a vial with 1.2 mL of PBS and sonicated at 40 kHz for 2 min to remove bacteria. The sonicated solution was 10-fold serially diluted and 100 μL of the pure solution and dilutions (up to 10-3) plated on blood, chocolate, and Sabouraud agar (Becton Dickinson) plates in triplicate. The blood and chocolate agar plates were incubated in a 5% CO₂ incubator at 37° C. The Sabouraud agar plates were incubated at 37° C. in atmospheric air. The number of colonies forming units per mL was determined after incubation for 24 hours. Different colonies were sampled and re-streaked on new plates for DNA extraction and sequence-based identification.

Bacterial colonies of interest sampled from the in vivo assay plates were grown on a separate plate of the same type they were found on for an additional 24 hours. The 16S rDNA sequence was amplified using primers 8F and 1493R, which flank all 16S variable regions. Amplified products were purified and sequenced (Genewiz). The obtained sequences were aligned and edited using Geneious 8.0. Sequence identity was searched in GenBank using the BLAST (blastn algorithm) program with default parameters.

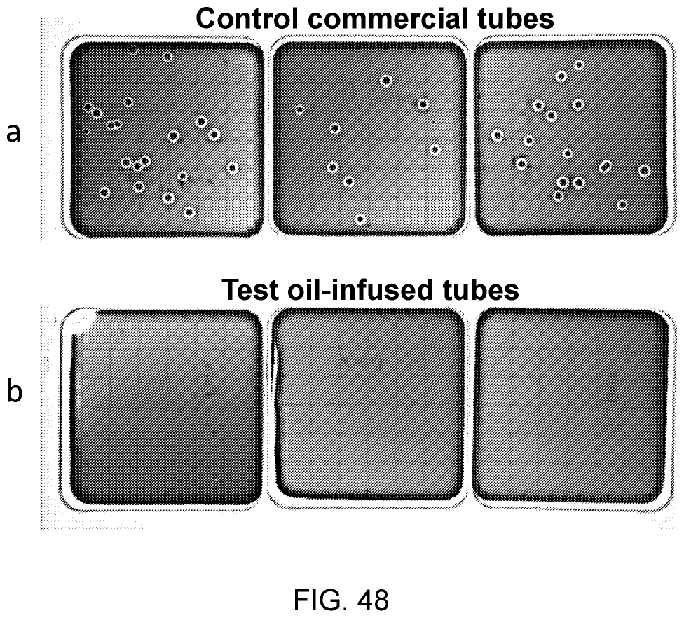

FIG. 48 depicts a comparative study of bacterial adhesion to commercial control silicone tube and medical grade silicone MED4960 infused in medical grade 100 cP silicone oil, demonstrating absence of bacteria-forming units of S. aureus (identified via sequencing) to liquid-infused silicone sheets, as shown in the photographs of the agar plates.

### II. Materials

A. Conduit Materials

Polymers that can be used for forming the tube include without limitation biostable or bioabsorbable polymers, according to certain embodiments. Non-limiting examples include isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, chitins, and combinations thereof.

Other non-limiting examples of polymers that can be used for forming the tubes, or for example the tubes used as stents, include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, gelatin, collagen, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and combinations thereof.

In certain embodiments, tubes can also be made with non-polymers. Non-limiting examples of useful non-polymers include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof sphingosine and derivatives thereof sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Particularly useful non-polymers include cholesterol, glyceryl monostearate, glycerol tristearate, stearic acid, stearic anhydride, glyceryl monooleate, glyceryl monolinoleate, acetylated monoglycerides, and combinations thereof.

The materials for the conduit designs listed in these embodiments can be selected from a group consisting of FDA-approved materials, such as silicones and fluoroplastics, Nylon, polyethylene terephthalate, Polycarbonate, Acrylonitrile Butadiene Styrene, Poly(p-phenylene oxide), Polybutylene terephthalate, Acetal, Polypropylene, Polyurethane, Polyetheretherketone, hydroxylpatite, Ultra-high molecular weight polyethylene, High Density Polyethylene, Low Density Polyethylene, Polystyrene High Impact, Polysulfone, Polyvinylidene fluoride, polystyrene, polymethylmethacrylate, latex, polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, and mixtures thereof. In addition, they can include polyelectrolyte hydrogels: ionic (including anionic or cationic) and ampholytic (including both anionic and cationic), for which incorporating more hydrophilic or hydrophobic monomers in hydrogel composition would allow for regulation of the volume transition behavior of the hydrogel. Non-limiting examples include hydrogel-forming materials such acrylate, polyacrylate, methacrylic acid, (dimethylamino)ethyl methacrylate, hydroxyethyl methacrylate, poly(vinyl alcohol)/poly(acrylic acid), 2-acrylamido-2-methylpropane sulfonic acid, [(methacrylamido)-propyl]trimethyl ammonium chloride, poly(N-vinyl-2-pyrrolidone/itaconic acid). Another category of materials can be represented by nonionic hydrogels. Non-limiting examples include poly(ethylene glycol), ethylene glycol diacrylate, polyethylene glycol diacrylate poly(ethylene oxide), diacrylate, acrylamide, polyacrylamide, methylenebisacrylamide, N-isopropylacrylamides, poly(vinyl alcohol) and mixtures thereof. In some embodiments, the hydrogel can be made of natural materials, such as proteins (e.g. collagen and silk) and polysaccharides (e.g. chitosan, dextran and alginate), and combinations thereof. In some embodiments, the tubes can be made of metals or metal oxides.

In certain embodiments, the materials can also contain colloidal particles that are dispersed or suspended in another substance. Non-limiting examples of suitable colloidal particles that can be used in the hydrogel-based sensors include polystyrene and polymethylmethacrylate, melamine resins (having a large number of reactive amino and imino groups for immobilization of different metal ions or metal nanoparticles), silica and polydivinylbenzene microparticles. In some embodiments the colloidal particles are made of one or more of the following polymers: poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, other halogenated polymers, hydrogels, organogels, or combinations thereof. Other polymers of different architectures can be utilized as well, such as random and block copolymers, branched, star and dendritic polymers, and supramolecular polymers. In certain embodiments, the colloidal particles are of natural origin (biopolymer colloid), such as a protein- or polysaccharide-based material, silk fibroin, chitin, shellac, cellulose, chitosan, alginate, gelatin, or a mixture thereof. In certain embodiments, the colloidal particles include one or more metals, such as gold, palladium, platinum, silver, copper, rhodium, ruthenium, rhenium, titanium, osmium, iridium, iron, cobalt, or nickel, or a combination thereof. In certain embodiments, the colloidal particles include one or more oxides, such as silica, alumina, beryllia, noble metal oxides, platinum group metal oxides, titania, tin oxide, zirconia, hafnia, molybdenum oxide, tungsten oxide, rhenium oxide, vanadium oxide, tantalum oxide, niobium oxide, chromium oxide, scandium oxide, yttria, lanthanum oxide, ceria, thorium oxide, uranium oxide, other rare earth oxides, or a combination thereof. Other class of particles to include is ferromagnetic, ferrimagnetic or superparamagnetic particles (diameter usually 10 nanometers or less). Exemplary nanoparticles include iron, nickel and cobalt containing particles, such as magnetite or hematite, Colloidal particles useful in the conduits described herein can be charged, or uncharged, hydrophilic, hydrophobic, or amphiphilic. In some embodiments, the conduits can contain two or more colloidal particles.

In any of these preceding embodiments, the precursor composition can comprise one or more additives selected from the group consisting small molecules, dispersed liquid droplets, or microparticle fillers, nanoparticle fillers, such as anti-oxidants, UV stabilizers, plasticizers, anti-static agents, porogens, slip agents, processing aids, foaming or antifoaming agents, nucleating agents and fillers to enhance mechanical properties or roughness, and to control optical properties or viscosity and uniformity of application, according to certain embodiments.

In certain embodiments, for medical and non-medical fluidic applications, the materials for the conduit designs listed in this innovation can include metals selected from the group of Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Ti, Pb, Bi, La, Ce, Pr, Nd, Sm, Eu, Gd, Tb, Dy, Ho, Er, Tm, Yb, Lu, and their oxides or a combination thereof. In certain embodiments, the metal-containing conduit contains aluminum and the roughened metal-containing surface contains boehmite. In certain embodiments, the metal-containing sol-gel precursor contains a porogen.

The materials for the conduit designs can include metal foams or porous metallic substrates. In certain embodiments, these porous substrates can be formed typically by the solidification process of a mixture of pre-melted metals with injected gas/gas-releasing blowing agents, or by compressing metal powders into special tooling to form different shapes and forms (e.g., sheet, cylindrical shape, hollow cylinders etc.). Metal foams can be manufactured either in closed-cell or open-cell structures (i.e., interconnected network of metals). Metal foams of different materials, such as aluminum, titanium, nickel, zinc, copper, steel, iron, or other metals and alloys, can be used, and have been produced by various methods, such as direct foaming and powder compact melting methods, which have been extensively discussed in J. Banhart, Prog. Mater. Sci 46, 559-632 (2001), which is incorporated herein by reference.

B. Surface Properties

A range of surface structures with different feature sizes and porosities can be used for conduit design, according to certain embodiments. Feature sizes can be in the range of hundreds of nanometers to microns (e.g., 100 to 1000 nm), and have aspect ratios from about 1:1 to 10:1. In certain embodiments, the surface has a large surface area that is readily wetted by the lubricating liquid and which entrains lubricating liquid and retains it on the substrate surface. The roughened surface material can be selected to be chemically inert to the lubricating liquid and to have good wetting properties with respect to lubricating liquid. In addition, the roughened surface topographies can be varied over a range of geometries and size scale to provide the desired interaction, e.g., wettability, with lubricating liquid. In certain embodiments, the roughened surface can be the surface of a three-dimensionally porous material. The porous material can be any suitable porous network having a sufficient thickness to stabilize lubricating liquid, such as a thickness from about 5 μm to about 1 mm. Moreover, the porous material can have any suitable pore sizes to stabilize the lubricating liquid, such as from about 10 nm to about 100 μm.

In other embodiments, a roughened surface is further functionalized to improve wetting by lubricating liquid. Surface coating can be achieved by methods well known in the art, including plasma assisted chemical vapor deposition, chemical functionalization, solution deposition, and vapor deposition. For example, surfaces containing hydroxyl groups (i.e., —OH) can be functionalized with various commercially available fluorosilanes (e.g., (1H,1H,2H,2H-tridecafluorooctyl)-trichlorosilane) to improve wetting by low surface tension fluids. In certain embodiments, many materials having native oxides can be activated to contain —OH functional groups using techniques such as plasma treatment. After activation, either vapor or solution deposition techniques can be used to attach silanes so that surfaces with low surface energy can be produced. For vapor deposition, the deposition can be carried out by exposing the surface to silane vapors. For solution deposition, the deposition can be carried out by immersing the surface in a silane solution, followed by rinsing and blow-drying after deposition. For layered deposition, layered deposition of a primer is followed by application of a mixture of sacrificial beads and the lubricating liquid, which is dried and cured. The beads are removed to produce a contiguous porous surface.

In certain embodiments, the roughened surface can have pores that are comparable or smaller than the material to be repelled. For example, pore sizes that are smaller than the size of protozoa (e.g., 10 μm), bacteria (e.g., 1 μm), viruses (e.g., 0.1 μm), and the like can be utilized.

C. Lubricating Liquids

Lubricating liquid can be selected from a number of different fluids. These fluids can be selected based on their suitability for biocompatibility, low toxicity, anti-fouling performance, drug release and chemical stability under physiological conditions. In one or more aspects, the lubricating liquid is a chemically inert, high-density biocompatible fluid, non-limiting examples of which include castor oil, silicone oil, fluocinolone acetonide oil, olive oil and mineral oil.

The lubricating liquid infiltrates, wets, and stably adheres to the substrate. Moreover, it is chemically inert with respect to the solid substrate and the fluid to be repelled. The lubricating liquid is non-toxic. Further, the lubricating liquid in accordance with certain aspects is capable of repelling immiscible fluids of any surface tension. In one or more aspects, the lubricating liquid is a chemically-inert and high-density biocompatible fluid. Further, the lubricating liquid is capable of repelling immiscible fluids, and in particular biological fluids of any surface tension. For example, the enthalpy of mixing between the fluid to be repelled and lubricating liquids be can be sufficiently high (e.g., water and oil) that they phase separate from each other when mixed together. In one or more embodiments, lubricating liquid is inert with respect to the solid surface and biological fluid. Lubricating liquid flows readily into the recesses of the roughened surface and generally possesses the ability to form an ultra-smooth surface when provided over the roughened surface. Some exemplary suitable lubricating liquid includes perfluorinated hydrocarbons, organosilicone compound (e.g. silicone elastomer), hydrophobic materials, and the like. In particular, the tertiary perfluoroalkylamines (such as perfluorotri-npentylamine, FC-70 by 3M, perfluorotri-n-butylamine FC-40, etc), perfluoroalkylsulfides and perfluoroalkylsulfoxides, perfluoroalkylethers, perfluorocycloethers (like FC-77) and perfluoropolyethers (such as KRYTOX family of lubricants by DuPont), perfluoroalkylphosphines and perfluoroallcylphosphineoxides as well as their mixtures can be used for these applications, as well as their mixtures with perfluorocarbons and any and all members of the classes mentioned. In addition, long-chain perfluorinated carboxylic acids (e.g., perfluorooctadecanoic acid and other homologues), fluorinated phosphonic and sulfonic acids, fluorinated silanes, and combinations thereof can be used as the lubricating liquid. The perfluoroalkyl group in these compounds could be linear or branched and some or all linear and branched groups can be only partially fluorinated. In certain embodiments, hydrophobic materials such as olive oil, silicone oil, hydrocarbons, and the like can be utilized as the lubricating liquid. In certain embodiments, ionic liquids can be utilized as the lubricating liquid.

In certain embodiments, the lubricating liquids used to facilitate repellency are selected to create a fluid surface that is intrinsically smooth, stable, and defect free. The lubricating liquid of certain embodiments infiltrate, wet, and stably adhere to the substrate. Moreover, the lubricating liquid of certain embodiments should be chemically inert with respect to the solid substrate and the fluid to be repelled. The lubricating liquid of certain embodiments should provide for adequate release of the drug and be non-toxic. Further, the lubricating liquid in accordance with certain aspects is capable of repelling immiscible fluids of any surface tension. In one or more aspects, the lubricating liquid is a chemically-inert and high-density biocompatible fluid.

Lubricating liquid can be selected from a number of different fluids according to certain embodiments. These fluids can be selected based on their suitability for drug release, biocompatibility, low toxicity, anti-clotting performance, and chemical stability under physiological conditions. In one or more aspects, the lubricating liquid is a chemically inert, high-density biocompatible fluid, non-limiting examples of which include vegetable oils. Vegetable oil refers to oil derived from plant seeds or nuts. Exemplary vegetable oils include, but are not limited to, almond oil, borage oil, black currant seed oil, castor oil, corn oil, safflower oil, soybean oil, sesame oil, cottonseed oil, peanut oil, olive oil, rapeseed oil, coconut oil, palm oil, canola oil, etc. Vegetable oils are typically “long-chain triglycerides,” formed when three fatty acids (usually about 14 to about 22 carbons in length, with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of oil-in-water emulsions. In certain embodiments, hydrogenated vegetable oils, which are produced by controlled hydrogenation of the vegetable oil, can be used in the systems disclosed herein.

Other oils can also be used but it can be necessary to modify the composition to provide for adequate solubilization of the drug in the oil. For example, perfluorinated hydrocarbons or organosilicone compound (e.g. silicone elastomer) and the like can be utilized. In particular, in certain embodiments the tertiary perfluoroalkylamines (such as perfluorotri-n-pentylamine, FC-70 by 3M, perfluorotri-n-butylamine FC-40, etc), perfluoroalkylsulfides and perfluoroalkylsulfoxides, perfluoroalkylethers, perfluorocycloethers (like FC-77) and perfluoropolyethers (such as KRYTOX family of lubricants by DuPont), perfluoroalkylphosphines and perfluoroalkylphosphineoxides as well as their mixtures can be used for these applications, as well as their mixtures with perfluorocarbons and any and all members of the classes mentioned. In addition, long-chain perfluorinated carboxylic acids (e.g., perfluorooctadecanoic acid and other homologues), fluorinated phosphonic and sulfonic acids, fluorinated silanes, and combinations thereof can be used as lubricants in certain embodiments. The perfluoroalkyl group in these compounds could be linear or branched and some or all linear and branched groups can be only partially fluorinated in certain embodiments. To improve drug solubility in these other oils, surfactants can be included in the compositions in certain embodiments.

For applications in certain non-medical applications, the lubricant can be selected from the group consisting of fluorinated lubricants (liquids or oils), silicones, mineral oil, plant oil, water (or aqueous solutions including physiologically compatible solutions), ionic liquids, polyolefins, including polyalpha-olefins (PAO), synthetic esters, polyalkylene glycols (PAG), phosphate esters, alkylated naphthalenes (AN) and silicate esters or any mixture thereof.

In certain embodiments, the lubricant has a high density. For example, lubricant that has a density that is more than 1.0 g/cm³, 1.6 g/cm³, or even 1.9 g/cm³ can be used.

In certain embodiments, the lubricant has a low freezing temperature, such as less than −5° C., −25° C., or even less than −80° C. Having a low freezing temperature will allow the lubricant to maintain its slippery behavior at reduced temperatures and to repel a variety of liquids or solidified fluids.

In certain embodiments, the lubricant can have a low evaporation rate, such as less than 1 nm/s, less than 0.1 nm/s, or even less than 0.01 nm/s. Taking a typical thickness of lubricant to be about 10 μm and an evaporation rate of about 0.01 nm/s, the surface can remain highly liquid-repellant for a long period of time without any refilling mechanisms.

In certain embodiments, the viscosity of the oil is in the range of about 1 to 2000 cSt. In certain embodiments, the viscosity of the oil is in the range of about 1 to 500 sCt.

In certain embodiments, the viscosity of the oil is in the range of about 8 to 1500 cSt. In certain embodiments, the viscosity of the oil is in the range of about 10 to 550 cSt. In certain embodiments, the viscosity of the oil is in the range of about 8 to 80 cSt. In certain embodiments, the viscosity of the oil is in the range of about 8 to 350 cSt. In certain embodiments, the viscosity of the oil is in the range of about 80 to 350 cSt. In certain embodiments, the viscosity of the oil is in the range of about 80 to 550 cSt

D. Stimuli-Responsive Materials

The simuli-responsive valves for the conduit lumen or the conduits themselves can comprise a nematic, smectic, chiral, dicotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

In one embodiment, the shape-responsive layer comprises a liquid crystal elastomer. Shape-changes in monodomain LCEs, which have a uniformly aligned liquid crystal (LC) director, can range from 10% to 400% of the initial LCE size. In some embodiments, the LCE is a polydomain liquid crystal elastomer. In some embodiments, the LCE includes a nematic director and a mesogen (liquid crystal molecule) associated with a polymer. In some embodiments, the mesogen content of the LCE ranges from about 20% molar content to about 90% molar content of the liquid crystal elastomer. In some embodiments, the mesogen is generally a molecule that produces a liquid crystal phase at room temperature and can include at least one of aromatic rings, aliphatic rings, poly aromatic rings, poly aliphatic rings, phenyls, biphenyls, cyanobiphenyls, benzenes, and combinations thereof. In some embodiments, the mesogen is functionalized with one or more functional groups, such as alkenes, alkanes, alkynes, carboxyl groups, esters, halogens, and combinations thereof. In certain embodiments, the mesogen is 4-methoxyphenyl 4-(3-butenyloxy) benzoate.

In some embodiments, mesogens in LCEs are cross-linked polymers. In some embodiments, the polymer includes at least one of polysiloxanes, poly(methyl) siloxanes (PMS), poly(dimethyl) siloxanes (PDMS), polymethylhydrosiloxane (PMHS), poly(methyl methacrylate), polyethylene, polypropylene, poly(butylacrylate) network chains and combinations thereof.

The polymers can be associated with mesogens in various arrangements. For instance, in some embodiments, the mesogens can be cross-linked to polymers. The crosslinker can be any reactive molecule that produces a physically or chemically crosslinked, elastomeric network. For example, a di(methacrylate) crosslinker is used or a diacrylate crosslinker. The crosslinker concentration can be varied to increase or decrease the elastomer modulus, at higher or lower crosslinker contents, respectively. Other catalysts or methods can be used to crosslink the network, including thermal annealing or platinum catalysts that are more or less reactive. The solvent content can also be varied during synthesis.

In some embodiments, a plurality of mesogens can be covalently coupled to a single polymer chain. In some embodiments, a plurality of mesogens can be covalently coupled to multiple polymer chains. In some embodiments, the mesogens and polymers can be intertwined within a matrix. LCEs can be made using methods known in the art.

In yet another embodiment, conductive material can be added to the shape-responsive layer. The conductive filler can provide the LCE nanocomposite with an electrical, magnetic, or light-induced response, as examples. For example, the LCE can comprise one or more wires. Alternatively or in addition to, carbon nanoconduits, carbon black nanoparticles, or conductive gold nanoparticles can be used.

In addition to tympanostomy conduits, the embodiments of the present disclosure can also enhance the field of other conduit-like medical implants, such as but not limited to surgical drains, vascular stents, catheter, dialysis tubing, feeding conduits, colostomy conduits, and eustachian implants.

1-104. (canceled)

105. A device comprising:
a conduit comprising
a proximal end, the proximal end having a proximal end radius,
a distal end opposite the proximal end, the distal end having a distal end radius,
an inner surface connecting the proximal end and the distal end, the inner surface forming a proximal angle at the proximal end and a distal angle at the distal end, the inner surface comprising surface properties, and
an outer surface connecting the proximal end and the distal end;
wherein distal end radius, the proximal end radius, the distal angle, the proximal angle, and the surface properties of the inner surface are selected to:
allow entry of a first material to the distal end of the conduit, allow transport of the first material through the conduit along the inner surface toward the proximal end, and allow exit of the first material from the proximal end of the conduit, and
resist entry of a second material into the proximal end of the conduit; and
wherein the Young-Laplace pressure for the first material is less than Young-Laplace pressure for the second material.

106. The device of claim 105, wherein the difference between the Young-Laplace pressure of the first material and the Young-Laplace pressure of the second material is in the range of 1 and 1,000 Pa.

107. The device of claim 105, wherein at least one of an angle or a surface property of the inner surface vary to maintain a substantially constant or reducing Young-Laplace pressure of the first material from the distal end to the proximal end or wherein at least one of an angle or a surface property of the inner surface varies such that there is substantially no pinning of the first material from the distal end.

108. The device of claim 105, wherein an advancing angle of the first material at the distal end as the first material enters the distal end is less than 90° and an advancing angle of the second material at the proximal end is as the second material enters the proximal end is greater than 90°.

109. The device of claim 105, wherein the shape of the conduit is selected from a group consisting of cylindrical, conical, and curved.

110. The device of claim 105, wherein the diameter of the proximal end is greater than the diameter of the distal end.

111. The device of claim 105, wherein the conduit comprises at least one of a distal flange disposed on the distal end of the conduit and a proximal flange disposed on the proximal flange of the conduit.

112. A device of claim 105, further comprising:
a portion of the conduit provided with a slippery surface comprising:
a partially or fully stabilized lubricating liquid layer on at least a portion of the inner surface or the outer surface of the conduit, the lubricating liquid layer wetting and adhering to at least a portion of the conduit to form the slippery surface over the portion of the conduit;

113. The device of claim 112, wherein the lubricating liquid decreases an advancing angle of the first material and increases an advancing angle of the second material.

114. The device of claim 112, wherein the lubricating liquid decreases the effective surface tension of the first material and increases the effective surface tension of the second material.

115. The device of claim 112, wherein the lubricating liquid is on at least one of the inner surface of the conduit, the outer surface of the conduit, the inner surface of the proximal flange, and the inner surface of the distal flange.

116. The device of claim 112, wherein the lubricating liquid is one or more of silicone oil, partially or fully fluorinated oil, mineral oil, carbon-based oil, castor oil, fluocinolone acetonide oil, food-grade oil, water, surfactant/surfactant solution, organic solvent, perfluorinated hydrocarbons, as well as mixtures thereof.

117. The device of claim 105, wherein the surface properties comprise a gradient or pattern on at least a portion of the conduit.

118. The device of claim 117, wherein the gradient or pattern is a chemical gradient or pattern, a geometric gradient or pattern, or a combination thereof.

119. The device of claim 117, wherein the gradient or pattern is on at least one of the inner surface of the conduit, the outer surface of the conduit, the inner surface of the proximal flange, and the inner surface of the distal flange.

120. The device of claim 117, wherein the gradient or pattern decreases the effective surface tension of the first material when the first material is disposed on the gradient or pattern and increases the effective surface tension of the second material when the second material is disposed on the gradient or pattern.

121. The device of claim 117, wherein the gradient or pattern is selected from a group consisting of geometrically patterned channels, macro-porous channels, micro-porous channels, three-dimensional periodic networks of pores, sponge-like networks of pores, surface roughness, grooves, ridges, indentations, micropillars, and microridges.

122. The device of claim 105, wherein the conduit comprises a stimulus-responsive portion, the stimulus being selected from one or more of light, temperature, pressure, electric field, magnetic field, swelling, de-swelling, pH, a lubricating liquid, a chemical composition.

123. The device of claim 122, wherein the stimulus-responsive portion comprises a flange disposed at or near the proximal end or the distal end of the conduit; and
wherein the flange is capable of transitioning between a first configuration and a second configuration in response to the stimulus,
wherein the flange changes at least one of a size of the flange or a shape of the flange when transitioning between the first configuration and the second configuration.

124. The device of claim 122, wherein the stimuli responsive portion is a valve disposed within the conduit, the valve being capable of closing in response to the stimulus.

125. The device of claim 122, wherein
the conduit has a first diameter in the first configuration, and
the conduit has a second diameter in the second configuration.

126. The device of claim 122, wherein the stimuli-responsive portion is disposed on one of more of the inner surface of the conduit and the outer surface of the conduit.

127. The device of claim 122, wherein the conduit further comprises a lumen defined by the inner surface and extending from the distal end to the proximal end,
wherein the stimuli-responsive portion is disposed in the lumen,
wherein the lumen is open to the first material in the first configuration and closed to the first material in the second configuration.

128. The device of claim 105, wherein the conduit comprises a tube, and wherein the device further comprises a second conduit, the second conduit comprising a tube having a proximal end and a distal end, the second conduit proximal end disposed near the proximal end of the conduit and the second conduit distal end disposed near the distal end of the conduit.

129. The device of claim 105, wherein the distal end radius, the proximal end radius, the distal angle, the proximal angle, and the surface properties of the inner surface are selected to allow entry of a third material to the proximal end of the conduit, allow transport of the third material through the conduit along the inner surface toward the distal end, and resist exit of the third material from the proximal end of the conduit;
wherein the Young-Laplace pressure for the third material is less than the Young-Laplace pressure for the second material, but below the breakthrough pressure at the distal end.

130. The device of claim 129, wherein at least a portion of the inner surface is configured to pin the third material thereon.

131. The device of claim 129, wherein the difference between the Laplace pressure of the second material and the Laplace pressure of the third material is between 1 Pa and 1000 Pa.

132. The device of claim 129, wherein the distal end is configured to have breakthrough pressure of at least 1 Pa higher than the Young-Laplace pressure of the third material at the location of the distal end to prevent exit of the third material from the distal end.

133. The device of claim 129, wherein the advancing angle of the third material at the proximal end as the third material enters the proximal end is less than 90°.

134. The device of claim 105, wherein the distal end radius, the proximal end radius, the distal angle, the proximal angle, and the surface properties of the inner surface are selected to allow entry of a fourth material to the proximal end of the conduit, allow transport of the fourth material through the conduit along the inner surface toward the distal end, and allow exit of the first material from the distal end of the conduit; and
wherein the Young-Laplace pressure for the fourth material is less than the Yong-Laplace pressure for the second material.

135. The device of claim 134, wherein the difference between the Young-Laplace pressure of the second material and the Laplace pressure of the fourth material is in the range of 1 Pa to 1000 Pa.

136. The device of claim 134, wherein at least one of an angle or a surface property of the inner surface vary to maintain a substantially constant or reducing Young-Laplace pressure of the fourth material from the proximal end to the distal end or wherein at least one of an angle or a surface property of the inner surface varies such that there is substantially no pinning of the first material from the proximal end to the distal end.

137. The device of claim 134, wherein an advancing angle of the fourth material at the proximal end as the fourth material enters the proximal end is less than 90°.

138. The device of claim 134, wherein the distal end is configured to have breakthrough pressure for the fourth liquid of at least 1 Pa lower than the Young-Laplace pressure of the fourth liquid at the location of the distal end to enable its exit.

139. The device of claim 105, wherein the first material is selected from the group consisting of effusion, pus, blood, plasma, tears, breast milk, amniotic fluid, serum, synovial fluid, cerebrospinal fluid, urine, saliva, sputum, sweat, other bodily fluid, water, water containing surfactants, perilymph, endolymph, mucus, and any combination thereof.

140. The device of claim 105, wherein the second material is selected from the group consisting of water, aqueous solutions, foams and emulsions, ototoxic agents, soap, pool water, fresh water, salt-containing water, or precipitation, foams and emulsions, ototoxic agents.

141. The device of claim 129, wherein the third material is selected from a group consisting of lubricating liquids, cross-linkers, aqueous and oil-based solutions of antibiotics, antiseptics, anti-viral agents, anti-inflammatory agents, small molecules, immunologics, nanoparticles, genetic therapies including viral and lipid-based therapies, chemotherapeutics, stem cells, cellular therapeutics, growth factors, proteins, radioactive materials, other liquid or gas-based pharmaceutical compounds, and combinations thereof, cerumenolytic agents, e.g. squalene, chlorhexidine, and EDTA, deferoxamine, dihydroxybenzoic acid, glutathione, D methionine and N acetylcysteine, also in forms of foams and emulsions.

142. The device of claim 134, wherein the fourth material is selected from the group consisting of oil-based, water-based, and other solvent-based therapeutics containing at least one of antibiotics, antiseptics, anti-viral agents, anti-inflammatory agents, small molecules, immunologics, nanoparticles, air for ventilation, genetic therapies including viral and lipid based therapies, chemotherapeutics, stem cells, cellular therapeutics, growth factors, proteins, radioactive materials, other liquid or gas-based pharmaceutical compounds, and combinations thereof.

143. The device of claim 105, wherein the conduit comprises one or more of a hydrogel, a chemically crosslinked polymer, a supramolecular polymer, a metal, a metal oxide, a porous material, geometrically-patterned pores or channels in a material, membranes and sponges, colloid- and surfactant-templated pores, grooves and ridges, periodic and aperiodic arrays of indentations, nano- and microstructures: nanoforest, nanoscale patterned films, microplatelets, micropillars, and microridges.

144. The device of claim 105, wherein the conduit comprises one or more of biostable or bioabsorbable polymers, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins chitins, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, gelatin, collagen, or combinations thereof.

145. The device of claim 105, wherein the conduit includes one or more of Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Ti, Pb, Bi, La, Ce, Pr, Nd, Sm, Eu, Gd, Tb, Dy, Ho, Er, Tm, Yb, Lu, and their oxides.

146. A tympanostomy or ventilation device comprising:
the conduit of claim 105, configured to be positioned in an ear, the conduit comprising:
an input port at the distal end configured to be received in an ear canal, the input port configured to receive a first liquid;
an output port at the proximal end configured to be received in a middle ear, the output port configured to output the first liquid received in the input port;
an inner surface extending from the input port to the output port, at least a portion of the inner surface comprising a conical or curved geometry extending at least partially between the input port and the output port to allow the transport of the first liquid between the ports.
